SEQUENCE LISTING (31 sequences)

SEQ ID NO: 1 - protein, 293 aa, Homo sapiens [sequence omitted]

SEQ ID NO: 2 - protein, 271 aa, Homo sapiens
VTEHVLANNDVSCDHPSNTVPSGSNQDLGAGAGEDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNNLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 3 - protein, 293 aa, Artificial Sequence
  Description of Artificial Sequence Synthetic polypeptide
MATARPPWMWVLCALITALLLGVTEHVLANNDVSCDHPSNTVPSGSNQDLGAGAGEDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNDLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 4 - protein, 271 aa, Artificial Sequence
  Description of Artificial Sequence Synthetic polypeptide
VTEHVLANNDVSCDHPSNTVPSGSNQDLGAGAGEDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNDLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 5 - protein, 293 aa, Artificial Sequence
  Description of Artificial Sequence Synthetic polypeptide
MATARPPWMWVLCALITALLLGVTEHVLANNDVSCDHPSNTVPSGSNQDLGAGAREDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNNLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 6 - protein, 271 aa, Artificial Sequence
  Description of Artificial Sequence Synthetic polypeptide
VTEHVLANNDVSCDHPSNTVPSGSNQDLGAGAREDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNNLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 7 - protein, 293 aa, Artificial Sequence
  Description of Artificial Sequence Synthetic polypeptide
MATARPPWMWVLCALITALLLGVTEHVLANNDVSCDHPSNTVPSGSNQDLGAGAREDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNDLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 8 - protein, 271 aa, Artificial Sequence
  Description of Artificial Sequence Synthetic polypeptide
VTEHVLANNDVSCDHPSNTVPSGSNQDLGAGAREDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNDLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 9 - protein, 1064 aa, Homo sapiens
MKIATVSVLLPLALCLIQDAASKNEDQEMCHEFQAFMKNGKLFCPQDKKFFQSLDGIMFINKCATCKMILEKEAKSQKRARHLARAPKATAPTELNCDDFKKGERDGDFICPDYYEAVCGTDGKTYDNRCALCAENAKTGSQIGVKSEGECKSSNPEQDVCSAFRPFVRDGRLGCTRENDPVLGPDGKTHGNKCAMCAELFLKEAENAKREGETRIRRNAEKDFCKEYEKQVRNGRLFCTRESDPVRGPDGRMHGNKCALCAEIFKQRFSEENSKTDQNLGKAEEKTKVKREIVKLCSQYQNQAKNGILFCTRENDPIRGPDGKMHGNLCSMCQAYFQAENEEKKKAEARARNKRESGKATSYAELCSEYRKLVRNGKLACTRENDPIQGPDGKVHGNTCSMCEVFFQAEEEEKKKKEGKSRNKRQSKSTASFEELCSEYRKSRKNGRLFCTRENDPIQGPDGKMHGNTCSMCEAFFQQEERARAKAKREAAKEICSEFRDQVRNGTLICTREHNPVRGPDGKMHGNKCAMCASVFKLEEEEKKNDKEEKGKVEAEKVKREAVQELCSEYRHYVRNGRLPCTRENDPIEGLDGKIHGNTCSMCEAFFQQEAKEKERAEPRAKVKREAEKETCDEFRRLLQNGKLFCTRENDPVRGPDGKTHGNKCAMCKAVFQKENEERKRKEEEDQRNAAGHGSSGGGGGNTQDECAEYREQMKNGRLSCTRESDPVRDADGKSYNNQCTMCKAKLEREAERKNEYSRSRSNGTGSESGKDTCDEFRSQMKNGKLICTRESDPVRGPDGKTHGNKCTMCKEKLEREAAEKKKKEDEDRSNTGERSNTGERSNDKEDLCREFRSMQRNGKLICTRENNPVRGPYGKMHINKCAMCQSIFDREANERKKKDEEKSSSKPSNNAKDECSEFRNYIRNNELICPRENDPVHGADGKFYTNKCYMCRAVFLTEALERAKLQEKPSHVRASQEEDSPDSFSSLDSEMCKDYRVLPRIGYLCPKDLKPVCGDDGQTYNNPCMLCHENLIRQTNTHIRSTGKCEESSTPGTTAASMPPSDE

SEQ ID NO: 10 - protein, 1042 aa, Homo sapiens
KNEDQEMCHEFQAFMKNGKLFCPQDKKFFQSLDGIMFINKCATCKMILEKEAKSQKRARHLARAPKATAPTELNCDDFKKGERDGDFICPDYYEAVCGTDGKTYDNRCALCAENAKTGSQIGVKSEGECKSSNPEQDVCSAFRPFVRDGRLGCTRENDPVLGPDGKTHGNKCAMCAELFLKEAENAKREGETRIRRNAEKDFCKEYEKQVRNGRLFCTRESDPVRGPDGRMHGNKCALCAEIFKQRFSEENSKTDQNLGKAEEKTKVKREIVKLCSQYQNQAKNGILFCTRENDPIRGPDGKMHGNLCSMCQAYFQAENEEKKKAEARARNKRESGKATSYAELCSEYRKLVRNGKLACTRENDPIQGPDGKVHGNTCSMCEVFFQAEEEEKKKKEGKSRNKRQSKSTASFEELCSEYRKSRKNGRLFCTRENDPIQGPDGKMHGNTCSMCEAFFQQEERARAKAKREAAKEICSEFRDQVRNGTLICTREHNPVRGPDGKMHGNKCAMCASVFKLEEEEKKNDKEEKGKVEAEKVKREAVQELCSEYRHYVRNGRLPCTRENDPIEGLDGKIHGNTCSMCEAFFQQEAKEKERAEPRAKVKREAEKETCDEFRRLLQNGKLFCTRENDPVRGPDGKTHGNKCAMCKAVFQKENEERKRKEEEDQRNAAGHGSSGGGGGNTQDECAEYREQMKNGRLSCTRESDPVRDADGKSYNNQCTMCKAKLEREAERKNEYSRSRSNGTGSESGKDTCDEFRSQMKNGKLICTRESDPVRGPDGKTHGNKCTMCKEKLEREAAEKKKKEDEDRSNTGERSNTGERSNDKEDLCREFRSMQRNGKLICTRENNPVRGPYGKMHINKCAMCQSIFDREANERKKKDEEKSSSKPSNNAKDECSEFRNYIRNNELICPRENDPVHGADGKFYTNKCYMCRAVFLTEALERAKLQEKPSHVRASQEEDSPDSFSSLDSEMCKDYRVLPRIGYLCPKDLKPVCGDDGQTYNNPCMLCHENLIRQTNTHIRSTGKCEESSTPGTTAASMPPSDE

SEQ ID NO: 11 - protein, 1017 aa, Mus musculus
MKTATVPMLLTLAFYLTQDAAGEKGNQDPCMKFQAQMKNGTLTCPKGNNSSQSLNDIIFQSECILCKRALEQGAPTKIMNVKVLSRANRATDPAKLNCESFKQRRKDGDFICPSDTSSVCGTDGKTYRGRCELCAENAKSQNHVDVKSEGECGSSHLETDMCSDFRANVQDGRLGCTRESDPILGPDGRTHGNRCAMCAELFLKEAKENATRNRESRIRRDAEKELCKEFENQVRNGRLFCTRESDPIRGPDGKMHGNKCALCAEIFMRQFTEEKGKAEKNQKDAEERAKAKMEIQKRCSEFQDRARNGTLFCTRENDPIRGLDGKTHGNLCSMCQAFFKTEAEEKKAEAGSRNRRGSEESETYAKLCDEYRKARKNGQLYCTRENAPIRGPDGKIHGNTCSMCQAFFIQEDKARAKVKREAAKEMCSEFRNQARNGMLMCTRENDPVVGPDGKRHSNKCAMCASVFLLEEEEKKKDDKTEKVDAGKAKKEAVQELCRKYHTQLRNGPLRCTRRNNPIEGLDGKMYKNACFMCWAFFQQEAKKSGAGFRPKVKREVKVDCSEYLALSKRGEIFCTRENDPVRGPDGKTHGNKCAMCKAVFKKENEERKRKEGENQRITSGESSSGGNPKAKDECAQYRESMKHGQLSCTRESDPVRGVDGEHYNNKCVMCKELLQKEMEETNKNSASRSNGTGSATGKDVCDQFRSQMKNGKLLCTRESDPTRGPDGAMHGNKCAMCKERLEKEAAEKKKKEDEEKRNTETNKSDKEDKCHEYRSMQLDGRLICTRENDPVRDADGKMHVNKCAMCQMMFEREANERKMREENSRSQPTNEAKDQCGEVHNSVEDAKPRPARSSLPSIRGISKDECSEFQNLMKNEKLTCPETDDPVRGADGTFYQNKCHMCRDVLKNEAMKRSGLQEKSSDIRSTKEGDPEFSSSSRDSDMCKNYRILPRMGYLCPKNLNPVCGDDGQTYSNPCMLCHENLMRQTNTRIHNPGACEESSNLKTVSTGTPASEKMMQ

SEQ ID NO: 12 - protein, 995 aa, Mus musculus
EKGNQDPCMKFQAQMKNGTLTCPKGNNSSQSLNDIIFQSECILCKRALEQGAPTKIMNVKVLSRANRATDPAKLNCESFKQRRKDGDFICPSDTSSVCGTDGKTYRGRCELCAENAKSQNHVDVKSEGECGSSHLETDMCSDFRANVQDGRLGCTRESDPILGPDGRTHGNRCAMCAELFLKEAKENATRNRESRIRRDAEKELCKEFENQVRNGRLFCTRESDPIRGPDGKMHGNKCALCAEIFMRQFTEEKGKAEKNQKDAEERAKAKMEIQKRCSEFQDRARNGTLFCTRENDPIRGLDGKTHGNLCSMCQAFFKTEAEEKKAEAGSRNRRGSEESETYAKLCDEYRKARKNGQLYCTRENAPIRGPDGKIHGNTCSMCQAFFIQEDKARAKVKREAAKEMCSEFRNQARNGMLMCTRENDPVVGPDGKRHSNKCAMCASVFLLEEEEKKKDDKTEKVDAGKAKKEAVQELCRKYHTQLRNGPLRCTRRNNPIEGLDGKMYKNACFMCWAFFQQEAKKSGAGFRPKVKREVKVDCSEYLALSKRGEIFCTRENDPVRGPDGKTHGNKCAMCKAVFKKENEERKRKEGENQRITSGESSSGGNPKAKDECAQYRESMKHGQLSCTRESDPVRGVDGEHYNNKCVMCKELLQKEMEETNKNSASRSNGTGSATGKDVCDQFRSQMKNGKLLCTRESDPTRGPDGAMHGNKCAMCKERLEKEAAEKKKKEDEEKRNTETNKSDKEDKCHEYRSMQLDGRLICTRENDPVRDADGKMHVNKCAMCQMMFEREANERKMREENSRSQPTNEAKDQCGEVHNSVEDAKPRPARSSLPSIRGISKDECSEFQNLMKNEKLTCPETDDPVRGADGTFYQNKCHMCRDVLKNEAMKRSGLQEKSSDIRSTKEGDPEFSSSSRDSDMCKNYRILPRMGYLCPKNLNPVCGDDGQTYSNPCMLCHENLMRQTNTRIHNPGACEESSNLKTVSTGTPASEKMMQ

SEQ ID NO: 13 - protein, 500 aa, Artificial Sequence
  Description of Artificial Sequence Synthetic polypeptide
EAAKEICSEFRDQVRNGTLICTREHNPVRGPDGKMHGNKCAMCASVFKLEEEEKKNDKEEKGKVEAEKVKREAVQELCSEYRHYVRNGRLPCTRENDPIEGLDGKIHGNTCSMCEAFFQQEAKEKERAEPRAKVKREAEKETCDEFRRLLQNGKLFCTRENDPVRGPDGKTHGNKCAMCKAVFQKENEERKRKEEEDQRNAAGHGSSGGGGGNTQDECAEYREQMKNGRLSCTRESDPVRDADGKSYNNQCTMCKAKLEREAERKNEYGNSVTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 501 aa, Artificial Sequence
  Description of Artificial Sequence Synthetic polypeptide
EAAKEICSEFRDQVRNGTLICTREHNPVRGPDGKMHGNKCAMCASVFKLEEEEKKNDKEEKGKVEAEKVKREAVQELCSEYRHYVRNGRLPCTRENDPIEGLDGKIHGNTCSMCEAFFQQEAKEKERAEPRAKVKREAEKETCDEFRRLLQNGKLFCTRENDPVRGPDGKTHGNKCAMCKAVFQKENEERKRKEEEDQRNAAGHGSSGGGGGNTQDECAEYREQMKNGRLSCTRESDPVRDADGKSYNNQCTMCKAKLEREAERKNEYGNSVTSKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 268 aa, Homo sapiens
EAAKEICSEFRDQVRNGTLICTREHNPVRGPDGKMHGNKCAMCASVFKLEEEEKKNDKEEKGKVEAEKVKREAVQELCSEYRHYVRNGRLPCTRENDPIEGLDGKIHGNTCSMCEAFFQQEAKEKERAEPRAKVKREAEKETCDEFRRLLQNGKLFCTRENDPVRGPDGKTHGNKCAMCKAVFQKENEERKRKEEEDQRNAAGHGSSGGGGGNTQDECAEYREQMKNGRLSCTRESDPVRDADGKSYNNQCTMCKAKLEREAERKNEY

SEQ ID NO: 16 - protein, 520 aa, Artificial Sequence
  Description of Artificial Sequence Synthetic polypeptide
EAAKEMCSEFRNQARNGMLMCTRENDPVVGPDGKRHSNKCAMCASVFLLEEEEKKKDDKTEKVDAGKAKKEAVQELCRKYHTQLRNGPLRCTRRNNPIEGLDGKMYKNACFMCWAFFQQEAKKSGAGFRPKVKREVKVDCSEYLALSKRGEIFCTRENDPVRGPDGKTHGNKCAMCKAVFKKENEERKRKEGENQRITSGESSSGGNPKAKDECAQYRESMKHGQLSCTRESDPVRGVDGEHYNNKCVMCKELLQKEMEETNKNSASRSNGTGSAGNSRAQVTDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 17 - protein, 275 aa, Mus musculus
EAAKEMCSEFRNQARNGMLMCTRENDPVVGPDGKRHSNKCAMCASVFLLEEEEKKKDDKTEKVDAGKAKKEAVQELCRKYHTQLRNGPLRCTRRNNPIEGLDGKMYKNACFMCWAFFQQEAKKSGAGFRPKVKREVKVDCSEYLALSKRGEIFCTRENDPVRGPDGKTHGNKCAMCKAVFKKENEERKRKEGENQRITSGESSSGGNPKAKDECAQYRESMKHGQLSCTRESDPVRGVDGEHYNNKCVMCKELLQKEMEETNKNSASRSNGTGSA

SEQ ID NO: 18 - protein, 294 aa, Artificial Sequence
  Description of Artificial Sequence Synthetic polypeptide
REIVKLCSQYQNQAKNGILFCTRENDPIRGPDGKMHGNLCSMCQAYFQAENEEKKKAEARARGNSVTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 295 aa, Artificial Sequence
  Description of Artificial Sequence Synthetic polypeptide
REIVKLCSQYQNQAKNGILFCTRENDPIRGPDGKMHGNLCSMCQAYFQAENEEKKKAEARARGNSVTSKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 62 aa, Homo sapiens
REIVKLCSQYQNQAKNGILFCTRENDPIRGPDGKMHGNLCSMCQAYFQAENEEKKKAEARAR

SEQ ID NO: 21 - protein, 308 aa, Artificial Sequence
  Description of Artificial Sequence Synthetic polypeptide
MEIQKRCSEFQDRARNGTLFCTRENDPIRGLDGKTHGNLCSMCQAFFKTEAEEKKAEAGSRNRGNSRAQVTDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 22 - protein, 63 aa, Mus musculus
MEIQKRCSEFQDRARNGTLFCTRENDPIRGLDGKTHGNLCSMCQAFFKTEAEEKKAEAGSRNR

SEQ ID NO: 23 - protein, 86 aa, Homo sapiens
MRATAIVLLLALTLATMFSIECAKQTKQMVDCSHYKKLPPGQQRFCHHMYDPICGSDGKTYKNDCFFCSKVKKTDGTLKFVHFGKC

SEQ ID NO: 24 - protein, 67 aa, Homo sapiens
IECAKQTKQMVDCSHYKKLPPGQQRFCHHMYDPICGSDGKTYKNDCFFCSKVKKTDGTLKFVHFGKC

SEQ ID NO: 25 - protein, 299 aa, Artificial Sequence
  Description of Artificial Sequence Synthetic polypeptide
IESAKQTKQMVDCSHYKKLPPGQQRFCHREYDPICGSDGKTYKNDCFFCSKVKKTDGTLKFVHFGKCGNSVTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 300 aa, Artificial Sequence
  Description of Artificial Sequence Synthetic polypeptide
IESAKQTKQMVDCSHYKKLPPGQQRFCHREYDPICGSDGKTYKNDCFFCSKVKKTDGTLKFVHFGKCGNSVTSKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 312 aa, Artificial Sequence
  Description of Artificial Sequence Synthetic polypeptide
IESAKQTKQMVDCSHYKKLPPGQQRFCHREYDPICGSDGKTYKNDCFFCSKVKKTDGTLKFVHFGKCGNSRAQVTDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 28 - protein, 67 aa, Homo sapiens
IESAKQTKQMVDCSHYKKLPPGQQRFCHREYDPICGSDGKTYKNDCFFCSKVKKTDGTLKFVHFGKC

SEQ ID NO: 29 - protein, 4 aa, Artificial Sequence
  Description of Artificial Sequence Synthetic peptide
LLVY

SEQ ID NO: 30 - protein, 7 aa, Artificial Sequence
  Description of Artificial Sequence Synthetic peptide
EEAQGDK

SEQ ID NO: 31 - protein, 7 aa, Artificial Sequence
  Description of Artificial Sequence Synthetic peptide
APPIQSR

(19) United States
(12) Patent Application Publication
(10) Pub. No.: US 2019/0078160 A1
(43) Pub. Date: Mar. 14, 2019

(54) USE OF KLK5 ANTAGONISTS FOR TREATMENT OF A DISEASE

(71) Applicant: Genentech, Inc., South San Francisco, CA (US)

(72) Inventors: Amy Dressen, Palo Alto, CA (US); David B. Iaea, Mateo, CA (US); Moulay Hicham Alaoui Ismaili, San Mateo, CA (US); Janet K. Jackman, South San Francisco, CA (US); Robert A. Lazarus, South San Francisco, CA (US); Kelly Loyet, South San Francisco, CA (US); Henry R. Maun, Freiburg i. Br. (DE); Brian L. Yaspan, Pacifica, CA (US); Tangsheng Yi, South San Francisco, CA (US); Joseph R. Arron, San Mateo, CA (US); Hilda Y. Hernandez-Barry, Martinez, CA (US)

(73) Assignee: Genentech, Inc., South San Francisco, CA (US)

(21) Appl. No.: 15/958,696

(22) Filed: Apr. 20, 2018

Related U.S. Application Data
(60) Provisional application No. 62/488,515, filed on Apr. 21, 2017.

(51) Int. Cl. C12Q 1/6883

(52) U.S. Cl. CPC C12Q 1/6883; C12Q 2600/118; C12Q 2600/156; C12Q 2600/106; C12Q 2600/112

(57) ABSTRACT

Provided herein are methods of treating a subject, methods of predicting the response of a subject and selecting a subject suffering from a disease associated with KLK5, such as asthma or Netherton Syndrome. In particular, provided herein are uses of KLK5 antagonists for the treatment or diagnosis of asthma or Netherton Syndrome, such as an antibody or an Fc fusion polypeptide as well as pharmaceutical formulations comprising the same.

### CROSS-REFERENCE TO RELATED APPLICATIONS

This application claims priority to U.S. Provisional Patent Application Ser. No. 62/488,515 filed on Apr. 21, 2017, the entire contents of which are incorporated herein by reference.

### SEQUENCE LISTING

The instant application contains a Sequence Listing which has been submitted electronically in ASCII format and is hereby incorporated by reference in its entirety. Said ASCII copy, created on Apr. 18, 2018, is named P34247-US-1_SequenceListing.txt and is 94,144 bytes in size.

### FIELD OF THE INVENTION

Provided herein are methods of treating a subject, methods of predicting the response of a subject and selecting a subject suffering from a disease associated with KLK5, such as asthma or Netherton Syndrome. In particular, provided herein are uses of KLK5 antagonists for the treatment or diagnosis of asthma or Netherton Syndrome, such as an antibody or a binding polypeptide as well as pharmaceutical formulations comprising the same.

### BACKGROUND

Asthma is a clinically heterogeneous disorder associated with both genetic and environmental risk factors. Estimates of heritability from asthma twin studies vary from 35% to 80%, indicating an important role for genetic risk. See e.g., Ullemar et al., Allergy 71, 230-238 (2016). Several large scale GWAS have been performed for asthma and asthma related phenotypes, and many of the loci identified such as those near ORMDL3, IL13, IL1RL1 and TSLP genes have been confirmed in multiple study populations. See e.g., Bonnelykke et al., Nat Genet 46, 51-55 (2014). These studies have added to both the genetic underpinnings of the disease and the pathophysiology of asthma, but the common variants identified via published GWAS account for little of the overall genetic risk. This concept of the “missing heritability” has been discussed and debated in depth and has been hypothesized to be due to several factors including low power to detect gene-gene interactions, limited structural variation analysis, and the potential contribution of rare variation. See Manolio et al., Nature 461, 747-753 (2009). Another strategy for uncovering genetic predisposition to common disease is through selection of phenotypically similar subgroups, and it has been suggested that this strategy would be useful as we strive to more comprehensively understand asthma genetic architecture. See Bonnelykke and Ober, J Allergy Clin Immunol 137, 667-679 (2016). Genes that influence overall risk in asthmatics may contribute to separate and independent biologic processes, which, taken together, influence disease outcome. Homogenization of the study population through sub-phenotyping, while reducing sample size, may reveal variants that are enriched in that patient subset.

Several biomarkers of type 2 inflammation have been shown to be effective in defining those asthmatics where disease is driven by type 2 inflammation. See Wan, and Woodruff. Immunol Allergy Clin North Am 36, 547-557 (2016). The knowledge gained from these biomarkers has led to the identification of novel treatments which show improved efficacy in the asthmatic patients with type 2 inflammation driven disease. See Corren et al., N Engl J Med 365, 1088-1098 (2011). However, there is a dearth of knowledge surrounding type 2 low asthma and these patients will likely comprise the bulk of the unmet medical need in severe asthma going forward. See e.g., Arron et al., Clin Immunol 161, 11-22 (2015).

One of the downstream type 2 biomarkers, periostin, is secreted by bronchial epithelial cells and lung fibroblasts and is inducible by Th2 cytokines, including IL-13. See Takayama et al., J Allergy Clin Immunol 118, 98-104 (2006). Periostin is a predictive biomarker for enriched anti IL-13 (lebrikizumab) clinical response for patients with high levels of pre-treatment serum periostin; conversely, patients with low levels of pre-treatment serum periostin derived markedly less clinical benefit. See Corren et al., N Engl J Med 365, 1088-1098 (2011). As peripheral periostin levels are effective at defining an asthmatic subpopulation with differential treatment response, we hypothesized that this biomarker could also stratify a heterogeneous asthma study population to increase power in a genetic study. Most asthma GWAS have focused on the asthmatic population without regards to type 2 inflammation status.

Asthma identifies a broad spectrum of respiratory-related symptoms characterized by reversible airflow obstruction, bronchial hyper-responsiveness, and airway inflammation. Asthma severity varies greatly between patients and disease molecular heterogeneity among patients has been well documented. There is a need for improved treatments for asthma, particularly moderate-severe asthma with low levels of type 2 airway inflammation.

### SUMMARY

Provided herein are methods for treating asthma in a subject comprising administering an effective amount of a KLK5 antagonist to the subject.

Further provided herein are methods of predicting the response of a subject suffering from asthma to a treatment comprising a KLK5 antagonist, the method comprising (a) measuring the KLK5 level in a biological sample from the subject, (b) comparing the KLK5 level detected in the sample to a reference level, and (c) predicting that the subject will respond to the treatment when the KLK5 level measured in the sample is elevated compared to the reference level and predicting that the subject will not respond to the treatment when the KLK5 level measured in the sample is reduced compared to the reference level.

Further provided herein are methods of selecting a subject suffering from asthma for a treatment comprising a KLK5 antagonist, comprising determining the presence or absence of a genetic variation located in the KLK5 genomic sequence in a biological sample from the subject, wherein the presence of the genetic variation indicates that the subject is suitable for treatment with a KLK5 antagonist.

Further provided herein are methods for detecting the presence or absence of a genetic variation in the KLK5 genomic sequence indicating that a subject suffering from asthma is suitable for treatment with a KLK5 antagonist, comprising (a) contacting a sample from the subject with a reagent capable of detecting the presence or absence of the genetic variation located in the KLK5 genomic sequence; and (b) determining the presence or absence of the genetic variation, wherein the presence of the genetic variation indicates that the subject is suitable for treatment with a KLK5 antagonist.

In some embodiments of any of the methods, the asthma is associated with elevated levels of KLK5. In some embodiments of any of the methods, the asthma is associated with elevated levels of neutrophils. In some embodiments of any of the methods, the asthma is selected from the group consisting of type 2 low asthma, periostin low asthma and eosinophil low asthma. In some embodiments of any of the methods, the asthma is not associated with Netherton Syndrome. In some embodiments of any of the methods, the asthma is associated with reduced activity of SPINK5. In some embodiments of any of the methods, the asthma is not associated with one or more genetic variations in the gene encoding SPINK5 or a gene product thereof. In some embodiments of any of the methods, the treatment of the subject for asthma is based on the presence or absence of the genetic variation. In some embodiments of any of the methods, the asthma is related to a genetic variation located in the KLK5 genomic sequence. In some embodiments, the genetic variation is a SNP. In some embodiments, the genetic variation is SNP rs117639512.

In some embodiments of any of the methods, the KLK5 antagonist inhibits KLK5 by binding to the active site of KLK5. In some embodiments of any of the methods, the KLK5 antagonist inhibits KLK5 by binding to a binding region comprising one or more of the amino acid residues of KLK5 selected from the group consisting of the amino acid residues at position 108, 147, 150, 153, 168 and 245 of full-length unprocessed KLK5, i.e., including the signal peptide. In some embodiments of any of the methods, the KLK5 antagonist inhibits the serine protease activity of KLK5.

In some embodiments of any of the methods, the KLK5 antagonist is selected from the group consisting of an antibody, a binding polypeptide, a polynucleotide and a small molecule. In some embodiments, the antibody is a monoclonal antibody. In some embodiments, the antibody is a human, humanized, or chimeric antibody. In some embodiments, the antibody is a full length IgG1 antibody. In some embodiments, the antibody has an IC₅₀ of less than about 50 μM-1 μM, less than about 1 μM-500 nM, less than about 500 nM-100 nM, less than about 100 nM-10 nM, less than about 10 nM-1 nM, or less than about 1000 pM-100 pM. In some embodiments, the antibody has an IC₅₀ of less than about 10 nM-1 nM. In some embodiments, the antibody has an IC₅₀ of less than about 2 nM-1 nM. In some embodiments, the IC₅₀ is determined by a direct assay or coupled assay as described herein.

In some embodiments, the binding polypeptide is a KLK5 binding polypeptide. In some embodiments, the KLK5 binding polypeptide is a fusion polypeptide. In some embodiments, the fusion polypeptide is a SPINK fusion polypeptide. In some embodiments, the fusion polypeptide is a SPINK Fc fusion polypeptide. In some embodiments, the fusion polypeptide is a SPINK Fc fusion polypeptide. In some embodiments, the SPINK Fc fusion polypeptide comprises one or more domains of SPINK5. In some embodiments, the one or more domains of SPINK5 comprise SEQ ID NO:17 (E421-A695). In some embodiments, the one or more domains from SPINK5 comprise SEQ ID NO:22 (M293-R355). In some embodiments, the one or more domains from SPINK5 are from mouse origin. In some embodiments, the one or more domains of SPINK5 comprise SEQ ID NO:15 (E490-Y757). In some embodiments, the one or more domains from SPINK5 comprise SEQ ID NO:20 (R291-R352). In some embodiments, the one or more domains from SPINK5 are human origin. In some embodiments, the SPINK Fc fusion polypeptide comprises one domain of SPINK9. In some embodiments, the one domain of SPINK9 comprises SEQ ID NO:28 (120-C86.C22S.H48R.M49E). In some embodiments, the one domain of SPINK9 is from human origin.

In some embodiments, the small molecule is a protease inhibitor. In some embodiments, the protease inhibitor is leupeptin.

In some embodiments of any of the methods, the sample is selected from the group consisting of bronchial alveolar lavage, lung parenchyma, bronchial sub-epithelium, cerebrospinal fluid, blood, serum, sputum, saliva, mucosal scraping, tissue biopsy, lacrimal secretion, semen, or sweat.

Further provided herein is a KLK5 antagonist for use in medical treatment or diagnosis including therapy and/or treating of asthma.

Further provided herein is a SPINK fusion polypeptide. In some embodiments, the SPINK fusion polypeptide is a SPINK Fc fusion polypeptide. In some embodiments, the SPINK Fc fusion polypeptide inhibits the activity of KLK5. In some embodiments, the SPINK Fc fusion polypeptide comprises one or more domains of SPINK5. In some embodiments, the one or more domains of SPINK5 comprise SEQ ID NO:17 (E421-A695). In some embodiments, the one or more domains from SPINK5 comprise SEQ ID NO: 22 (M293-R355). In some embodiments, the one or more domains from SPINK5 are from mouse origin. In some embodiments, the one or more domains of SPINK5 comprise SEQ ID NO:15 (E490-Y757). In some embodiments, the one or more domains from SPINK5 comprise SEQ ID NO:20 (R291-R352). In some embodiments, the one or more domains from SPINK5 are human origin. In some embodiments, the SPINK Fc fusion polypeptide comprises one domain of SPINK9. In some embodiments, the one domain of SPINK9 comprises SEQ ID NO:28 (I20-C86.C22S.H48R.M49E). In some embodiments, the one domain of SPINK9 is from human origin.

In some embodiments, the SPINK fusion polypeptide has an IC₅₀ of less than about 50 μM-1 μM, less than about 1 μM-500 nM, less than about 500 nM-100 nM, less than about 100 nM-10 nM, less than about 10 nM-1 nM, or less than about 1000 pM-100 pM. In some embodiments, the SPINK fusion polypeptide has an IC₅₀ of less than about 10 nM-1 nM. In some embodiments, the SPINK fusion polypeptide has an IC₅₀ of less than about 3 nM-1 nM. In some embodiments, the IC₅₀ is determined by a direct assay or coupled assay as described herein.

Further provided herein is a SPINK fusion polypeptide as described herein for use in medical treatment or diagnosis including therapy and/or treating a disease associated with KLK5.

Further provided herein is a pharmaceutical formulation comprising a pharmaceutically active amount of a SPINK fusion polypeptide as described herein and a pharmaceutically acceptable carrier.

Further provided herein is a method for treating a disease associated with KLK5 in a subject comprising administering an effective amount of a SPINK fusion polypeptide as described herein to the subject.

In some embodiments of any of the SPINK fusion polypeptides, the disease associated with KLK5 is associated with elevated levels of KLK5 in a sample of the subject. In some embodiments, the disease associated with KLK5 is associated with elevated numbers of neutrophils in a sample of the subject. In some embodiments, the disease associated with KLK5 is Netherton Syndrome. In some embodiments, the sample is selected from the group consisting of bronchial alveolar lavage, lung parenchyma, and bronchial sub-epithelium. In some embodiments, the subject is a human.

### BRIEF DESCRIPTION OF THE FIGURES

FIGS. 1A and 1B. Comparison of periostin high (FIG. 1A) and periostin low (FIG. 1B) subgroups to controls. Loci were plotted by enrichment cohort. Eight loci showed no discernable difference and are not shown. For each locus, the OR was plotted and P-value in the case to control comparison was listed.

FIG. 2. Shows a summary of the genome-wide association results in the meta-analysis in the form of a Manhattan plot. A genome-wide single variant analysis in 667 adult non-type 2 inflammatory asthmatics and 1,887 controls was performed. The genome-wide significance level of P<5×10⁻⁸ is indicated by the upper line (marked by “X”), and suggestive significance (P<1×10⁻⁵) was indicated by the lower line (marked by “XX”).

FIG. 3. LocusZoom39 plot summarizing the result for the KLK locus on chromosome 19. The variants were color coded by the extent of linkage disequilibrium between them and rs117639512, the SNP of strongest association in the region.

FIGS. 4A and 4B. Increased KLK5 in asthma bronchial alveolar lavage independent of periostin level. FIG. 4A) Level of KLK5 binding polypeptide in bronchial alveolar lavage of healthy volunteer or severe asthma patients; FIG. 4B) Association of level of KLK5 and predicted FEV1 value in severe asthma patients.

FIGS. 5A and 5B. Recombinant KLK5 induces lung neutrophil extravasation and lung epithelium cytokine production. FIG. 5A) WT or SA mutant KLK5 (2 μg per mice) were intranasally delivered into mice and neutrophil cell number (quantified by Ly6G+CD11b+ cells) was quantified by flow cytometry analysis. FIG. 5B) Lung epithelial cells were treated with 2 μg/ml SA mutant or WT, or in the presence of 10 pig SPINK5 Fc fusion polypeptide. Transcripts of Tslp, Tnfa, IL-8, and Icam1 were quantified by real-time RT-PCR.

FIGS. 6A, 6B and 6C. Recombinant KLK5 activity is inhibited in direct assay by SPINK Fc fusion polypeptides. KLK5 was pre-incubated with SPINK5 M293-R355 (FIG. 6A), SPINK5 E421-A695 (FIG. 6B) or SPINK9 (I20-C86.C22S.H48R.M49E)-Fc (herein also referred to as SPINK9.SRE.Fc) (FIG. 6C) for 30 minutes prior to addition of fluorescent substrate, Boc-VPR-AMC. Reaction was monitored using a PHERAstar® Plus reader. The RFU/s reaction rate was calculated by linear regression of readings in the linear range. The IC₅₀ parameters were determined from a four-parameter fit for their respective curves.

FIGS. 7A, 7B and 7C. Recombinant KLK5 activity is inhibited in pro-KLK7 coupled assay by SPINK Fc fusion polypeptides. KLK5 was pre-incubated with, SPINK5 M293-R355 (FIG. 7A), SPINK5 E421-A695 (FIG. 7B) or SPINK9.SRE.Fc (FIG. 7C) for 30 minutes prior to addition of pro-KLK7 and fluorescent substrate, Suc-LLVY-AMC (SEQ ID NO:29). Reaction was monitored using a PHERAstar® Plus reader. The RFU/s reaction rate was calculated by linear regression of readings in the linear range. The IC₅₀ parameters were determined from a four-parameter fit for their respective curves.

FIGS. 8A, 8B, 8C and 8D. Recombinant KLK7 activity is partly inhibited by SPINK Fc fusion polypeptides but not SPINK9.SRE.Fc or mAb1108. KLK5 was pre-incubated with, SPINK5 M293-R355 (FIG. 8A), SPINK5 E421-A695 (FIG. 8B), SPINK9.SRE.Fc (FIG. 8C) or mAb1108 (FIG. 8D) for 50 minutes prior to addition of pro-KLK7 and fluorescent substrate, Suc-LLVY-AMC (SEQ ID NO:29). Reaction was monitored using a PHERAstar® Plus reader. The RFU/s reaction rate was calculated by linear regression of readings in the linear range. The IC₅₀ parameters were determined from a four-parameter fit for their respective curves.

FIGS. 9A, 9B, 9C and 9D. A commercial antibody, mAb1108, is a partial inhibitor of human KLK5. 20 nM (FIG. 9A), 10 nM (FIG. 9B), 5 nM (FIG. 9C) and 2.5 nM (FIG. 9D) KLK5 was incubated with mAb1108 for 30 minutes prior to addition of fluorescent substrate, Boc-VPR-AMC. Reaction was monitored using a PHERAstar® Plus reader. The RFU/s reaction rate was calculated by linear regression of readings in the linear range. The IC₅₀ value was determined from a four-parameter fit of the respective curves.

FIGS. 10A and 10B. SPINK9.SRE.Fc fusion protein is a potent inhibitor of KLK5 in the direct assay. KLK5 was incubated with SPINK9.SRE.Fc fusion (FIG. 10A) or mAb1108 (FIG. 10B) for 30 minutes prior to addition of fluorescent substrate, Boc-VPR-AMC. Reaction was monitored using a PHERAstar® Plus reader. The RFU/s reaction rate was calculated by linear regression of readings in the linear range. The IC₅₀ parameters were determined from a four-parameter fit for their respective curves.

FIGS. 11A and 11B. SPINK9.SRE.Fc fusion protein is a potent inhibitor of KLK5 in the pro-KLK7 coupled assay. In the pro-KLK7 coupled assay, KLK5 was incubated with SPINK9.SRE.Fc fusion (FIG. 11A) or mAb1108 (FIG. 11B) for 30 minutes prior to addition of pro-KLK7 and fluorescent substrate, Suc-LLVY-AMC (SEQ ID NO:29). Reaction was monitored using a PHERAstar® Plus reader. The RFU/s reaction rate was calculated by linear regression of readings in the linear range. The IC₅₀ value was determined from a four-parameter fit of the respective curves.

FIGS. 12A, 12B, 12C and 12D. SPINK9.SRE.Fc (FIGS. 12A and 12C) and mAb1108 (FIGS. 12B and 12D) dose-dependently inhibit recombinant KLK5 cleavage of the signal peptides from pro-KLK7 (FIGS. 12A and 12B) and pro-KLK1 (FIGS. 12C and 12D). The KLK7 and KLK1 signal peptides were detected by LC/MS. A pre-incubation of SPINK9.SRE.Fc or mAb1108 and KLK5 preceded a two-hour incubation of 5 nM KLK5 with 15 nM pro-KLK7 or a 20 minute incubation of 0.5 nM KLK5 with 300 nM (FIG. 12C) or 355 nM (FIG. 12D) pro-KLK1.

### DETAILED DESCRIPTION

Provided herein are methods of treating using KLK5 antagonists. In some embodiments, provided herein are methods of treating asthma using a KLK5 antagonist. In particular, provided herein are methods of treating asthma by administering an effective amount of a KLK5 antagonist to a subject. Also provided herein are methods of predicting a response of a subject or selecting a subject with asthma for treatment with a KLK5 antagonist based upon detecting the presence or absence of a genetic variation in KLK5. In some embodiments, provided herein are methods of treating Netherton syndrome using a KLK5 antagonist. In particular, provided herein are methods of treating Netherton syndrome using a KLK5 antagonist, wherein the KLK5 antagonist is a SPINK fusion polypeptide (e.g., SPINK Fc fusion polypeptide). Also provided herein are KLK5 antagonists for use in treatment or diagnosis of asthma as well as pharmaceutical formulations comprising the same.

### I. Definitions

The terms “KLK5” and “Kallikrein-5,” as used herein, refers to any native KLK5 from any vertebrate source, including mammals such as primates (e.g., humans) and rodents (e.g., mice and rats), unless otherwise indicated. The term encompasses “full-length,” unprocessed KLK5 as well as any form of KLK5 that results from processing in the cell. The term also encompasses naturally occurring variants of KLK5, e.g., splice variants or allelic variants. In some embodiments, the amino acid sequence of an exemplary human KLK5 is UNIPROT Q9Y337. In some embodiments, the amino acid sequence of an exemplary human KLK5 is selected from the group consisting of SEQ ID NO:1, SEQ ID NO:3 (N153D variant), SEQ ID NO:5 (G55R variant), and SEQ ID NO:7 (G55R, N153D variant). In some embodiments, the amino acid sequence of an exemplary human KLK5 is amino acid residues 23-293 (minus signal peptide) of UNIPROT Q9Y337 and is shown in SEQ ID NO:2. In some embodiments, the amino acid sequence of an exemplary human KLK5 is amino acid residues 23-293 (minus signal peptide) of the N153D variant shown in SEQ ID NO:4. In some embodiments, the amino acid sequence of an exemplary human KLK5 is amino acid residues 23-293 (minus signal peptide) of the G55R variant shown in SEQ ID NO:6. In some embodiments, the amino acid sequence of an exemplary human KLK5 is amino acid residues 23-293 (minus signal peptide) of the G55R, N153D variant shown in SEQ ID NO:8.

The numbering in this paragraph below, relates to full-length unprocessed KLK5. In some embodiments, the amino acid sequence of the human KLK5 comprises the amino acid N at position 153. In some embodiments, the amino acid sequence of the human KLK5 comprises the amino acid D at position 153. In some embodiments, the amino acid sequence of the human KLK5 comprises the amino acid G at position 55. In some embodiments, the amino acid sequence of the human KLK5 comprises the amino acid R at position 55. In some embodiments, the amino acid sequence of the human KLK5 comprises the amino acid G at position 55 and the amino acid N at position 153. In some embodiments, the amino acid sequence of the human KLK5 comprises the amino acid G at position 55 and the amino acid D at position 153. In some embodiments, the amino acid sequence of the human KLK5 comprises the amino acid R at position 55 and the amino acid N at position 153. In some embodiments, the amino acid sequence of the human KLK5 comprises the amino acid R at position 55 and the amino acid D at position 153.

The numbering in this paragraph below, relates to full-length unprocessed KLK5. In some embodiments, the nucleic acid sequence of the human KLK5 comprises a sequence encoding an N at position 153. In some embodiments, the nucleic acid sequence of the human KLK5 comprises a sequence encoding a D at position 153. In some embodiments, the nucleic acid sequence of the human KLK5 comprises a sequence encoding a G at position 55. In some embodiments, the nucleic acid sequence of the human KLK5 comprises a sequence encoding an R at position 55. In some embodiments, the nucleic acid sequence of the human KLK5 comprises a sequence encoding a G at position 55 and an N at position 153. In some embodiments, the nucleic acid sequence of the human KLK5 comprises a sequence encoding G at position 55 and a D at position 153. In some embodiments, the nucleic acid sequence of the human KLK5 comprises a sequence encoding R at position 55 and an N at position 153. In some embodiments, the nucleic acid sequence of the human KLK5 comprises a sequence encoding an R at position 55 and a D at position 153.

The terms “SPINK5” and “Serine protease inhibitor Kazal-type 5,” as used herein, refers to any native SPINK5 from any vertebrate source, including mammals such as primates (e.g., humans) and rodents (e.g., mice and rats), unless otherwise indicated. The term encompasses “full-length,” unprocessed SPINK5 as well as any form of SPINK5 that result from processing in the cell. The term also encompasses naturally occurring variants of SPINK5, e.g., splice variants or allelic variants. In some embodiments, the amino acid sequence of an exemplary human SPINK5 is UNIPROT Q9NQ38 and is shown in SEQ ID NO:9. In some embodiments, the amino acid sequence of an exemplary human SPINK5 is amino acid residues 23-1064 (minus signal peptide) of UNIPROT Q9NQ38 and is shown in SEQ ID NO:10. In some embodiments, the amino acid sequence of an exemplary mouse SPINK5 is UNIPROT Q5K5D4 and is shown in SEQ ID NO:11. In some embodiments, the amino acid sequence of an exemplary mouse SPINK5 is amino acid residues 23-1064 (minus signal peptide) of UNIPROT Q5K5D4 and is shown in SEQ ID NO:12.

The terms “SPINK9” and “Serine protease inhibitor Kazal-type 9,” as used herein, refers to any native SPINK9 from any vertebrate source, including mammals such as primates (e.g., humans) and rodents (e.g., mice and rats), unless otherwise indicated. The term encompasses “full-length,” unprocessed SPINK9 as well as any form of SPINK9 that result from processing in the cell. The term also encompasses naturally occurring variants of SPINK9, e.g., splice variants or allelic variants. In some embodiments, the amino acid sequence of an exemplary human SPINK9 is UNIPROT Q5DT21 and is shown in SEQ ID NO:23. In some embodiments, the amino acid sequence of an exemplary human SPINK9 is amino acid residues 20-86 (minus signal peptide) of UNIPROT Q5DT21 and is shown in SEQ ID NO:24.

An “antagonist of KLK5”, a “KLK5 antagonist”, an “inhibitor of KLK5” or a “KLK5 inhibitor” is an agent that interferes with activation or function of KLK5, e.g., partially or fully blocks, inhibits, or neutralizes a biological activity mediated by KLK5. For example, an antagonist of KLK5 may refer to any molecule that partially or fully blocks, inhibits, or neutralizes a biological activity mediated by KLK5. Examples of KLK5 antagonists include antibodies (e.g., anti-KLK5 antibodies), binding polypeptides (e.g., KLK5 binding polypeptides such as SPINK Fc fusion polypeptides), polynucleotides (e.g., KLK5 polynucleotide antagonists such as short interfering RNAs (siRNA) or clustered regularly interspaced short palindromic repeat RNAs (CRISPR-RNA or crRNA, including single guide RNAs (sgRNAs) having a crRNA and tracrRNA sequence), and small molecules (e.g., KLK5 small molecule antagonists such as small molecule protease inhibitors). In some embodiments, the antagonist is an antibody or small molecule which binds to KLK5.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The term “polypeptide” as used herein, refers to any native polypeptide of interest (e.g., KLK5, SPINK5 or SPINK9) from any vertebrate source, including mammals such as primates (e.g., humans) and rodents (e.g., mice and rats), unless otherwise indicated. The term encompasses “full-length,” unprocessed polypeptide as well as any form of the polypeptide that results from processing in the cell. The term also encompasses naturally occurring variants of the polypeptide, e.g., splice variants or allelic variants.

The term “SPINK fusion polypeptide” as used herein refers to a fusion polypeptide in which a SPINK polypeptide or a fragment thereof (e.g., certain domains of the SPINK polypeptide (e.g., SPINK5 and/or SPINK9) is linked, directly or indirectly, to another polypeptide (e.g., non-SPINK polypeptide).

The term “SPINK Fc fusion polypeptide” as used herein refers to a fusion polypeptide in which a SPINK polypeptide or a fragment thereof (e.g., certain domains of the SPINK polypeptide (e.g., SPINK5 and/or SPINK9) is linked, directly or indirectly, to an Fc region. In some embodiments, the Fc region is selected from the group consisting of an IgG1 Fc region, IgG2a Fc region and IgG4 Fc region. In some embodiments, the Fc region is an IgG2a Fc region. In some embodiments, the IgG2a Fc region is a mouse IgG2a Fc region. In some embodiments, the Fc region is an IgG1 Fc region. In some embodiments, the IgG1 Fc region is a human IgG1 Fc region. In some embodiments, the Fc region is an IgG4 Fc region. In some embodiments, the IgG4 Fc region is a human IgG4 Fc region. In some embodiments, the SPINK polypeptide or a fragment thereof is a human SPINK polypeptide or a fragment thereof. In some embodiments, the SPINK polypeptide or a fragment thereof is a mouse SPINK polypeptide or a fragment thereof. It is understood that minor sequence variations such as insertions, deletions, substitutions, especially conservative amino acid substitutions of the SPINK polypeptide, the SPINK domains or the Fc that do not affect the function and/or activity of the SPINK polypeptide, the SPINK domains or the SPINK Fc fusion polypeptide are provided herein. In some embodiments, the SPINK Fc fusion polypeptide provided herein can bind to KLK5, which can lead to inhibition of KLK5. In some embodiments, the SPINK polypeptide or a fragment thereof is SPINK 5. In some embodiments, the SPINK polypeptide or a fragment thereof is SPINK 9. The functions and/or activities of the SPINK Fc fusion polypeptide can be assayed by methods known in the art, including without limitation, ELISA, ligand-receptor binding assay and Stat3 luciferase assay.

In some embodiments, the Fc region of the SPINK Fc fusion polypeptide does not possess effector activities (e.g., does not bind to FcγIIIR) or exhibits substantially lower effector activity than a whole IgG antibody. In some embodiments, the Fc region of the SPINK Fc fusion polypeptide does not trigger cytotoxicity such as antibody-dependent cellular cytotoxicity (ADCC) or complement dependent cytotoxicity (CDC). Unless otherwise specified, “SPINK Fc fusion,” “SPINK Ig fusion polypeptide,” “SPINK Fc fusion polypeptide” or “SPINK Fc” are used interchangeably throughout this application.

The term “small molecule” refers to any molecule with a molecular weight of about 2000 daltons or less, preferably of about 500 daltons or less.

“Affinity” or “Binding Affinity” refers to the strength of the sum total of noncovalent interactions between a single binding site of a molecule (e.g., antibody, binding polypeptide, polynucleotide, small molecule) and its binding partner (e.g., an antigen). Unless indicated otherwise, as used herein, “binding affinity” refers to intrinsic binding affinity which reflects a 1:1 interaction between members of a binding pair (e.g., either of antibody, binding polypeptide, polynucleotide, small molecule and the antigen). The affinity of a molecule X for its partner Y can generally be represented by the dissociation constant (Kd). Affinity can be measured by common methods known in the art, including those described herein (e.g., peptide substrate assay, direct assay or coupled assay).

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

An “antibody that binds to the same epitope” or an “antibody that binds to the same binding region” as a reference antibody refers to an antibody that blocks binding of the reference antibody to its binding partner (e.g., an antigen) in a competition assay by 50% or more, and conversely, the reference antibody blocks binding of the antibody to its binding partner in a competition assay by 50% or more.

The terms “anti-KLK5 antibody” and “an antibody that binds to KLK5” refer to an antibody that is capable of binding KLK5 with sufficient affinity such that the antibody is useful as a diagnostic and/or therapeutic agent in targeting KLK5. In some embodiments, the extent of binding of an anti-KLK5 antibody to an unrelated polypeptide (polypeptide other than KLK5) is less than about 10% of the binding of the antibody to KLK5 as measured, e.g., by a radioimmunoassay (RIA). In some embodiments, an antibody that binds to KLK5 has a dissociation constant (Kd) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In some embodiments, an anti-KLK5 antibody binds to a binding region (e.g. an epitope) of KLK5 that is conserved among different species of KLK polypeptides.

A “blocking antibody” or an “antagonist antibody” is one which inhibits or reduces biological activity of the antigen it binds. Preferred blocking antibodies or antagonist antibodies substantially or completely inhibit the biological activity of the antigen.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

A “binding region” is the portion of the binding partner (e.g., an antigen) to which a KLK5 antagonist (e.g. antibodies, binding polypeptides, polynucleotides, small molecules) selectively binds. For a binding polypeptide binding partner, a linear binding region can be a peptide portion of about 4-15 (e.g., 4, 5, 6, 7, 8, 9, 10, 11, 12, 13, 14 or 15) amino acid residues. A non-linear, conformational binding region may comprise residues of a polypeptide sequence brought to close vicinity in the three-dimensional (3D) structure of the binding polypeptide binding partner.

The terms “full length antibody,” “intact antibody,” and “whole antibody” are used herein interchangeably to refer to an antibody (e.g., an anti-KLK5 antibody) having a structure substantially similar to a native antibody structure or having heavy chains that contain an Fc region.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In some embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:

(a) hypervariable loops occurring at amino acid residues 26-32 (L1), 50-52 (L2), 91-96 (L3), 26-32 (H1), 53-55 (H2), and 96-101 (H3). See Chothia and Lesk, J. Mol. Biol. 196:901-917 (1987);

(b) CDRs occurring at amino acid residues 24-34 (L1), 50-56 (L2), 89-97 (L3), 31-35b (H1), 50-65 (H2), and 95-102 (H3). See Kabat et al., Sequences of Proteins of Immunological Interest, 5th Ed. Public Health Service, National Institutes of Health, Bethesda, Md. (1991);

(c) antigen contacts occurring at amino acid residues 27c-36 (L1), 46-55 (L2), 89-96 (L3), 30-35b (H1), 47-58 (H2), and 93-101 (H3). See MacCallum et al. J. Mol. Biol. 262: 732-745 (1996); and

(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

The term “isolated” as used in reference to antibody, binding polypeptide, polynucleotide or small molecule is one which has been separated from a component of its natural environment. In some embodiments, an antibody, binding polypeptide, polynucleotide or small molecule is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC).

The term “monoclonal antibody” as used herein refers to an antibody obtained from a population of substantially homogeneous antibodies, i.e., the individual antibodies comprising the population are identical and/or bind the same binding region (e.g., epitope), except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies described herein may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies.

The term “variable region” or “variable domain” refers to the domain of an antibody heavy or light chain that is involved in binding the antibody to an antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

By “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed. With respect to the embodiment of polynucleotide analysis or protocol, one may use the results of the polynucleotide expression analysis or protocol to determine whether a specific therapeutic regimen should be performed.

“Elevated expression,” “elevated expression levels,” or “elevated levels” refers to an increased expression or increased levels of a biomarker in a subject relative to a control, such as a subject or subjects who are not suffering from the disease or disorder (e.g., asthma) or an internal control (e.g., housekeeping biomarker).

The term “housekeeping biomarker” refers to a biomarker or group of biomarkers (e.g., polynucleotides and/or polypeptides) which are typically similarly present in all cell types. In some embodiments, the housekeeping biomarker is a “housekeeping gene.” A “housekeeping gene” refers herein to a gene or group of genes which encode proteins whose activities are essential for the maintenance of cell function and which are typically similarly present in all cell types.

The term “KLK5 genomic sequence” as used herein, refers to either the cDNA and/or the genomic form of the KLK5 gene, which may include introns as well as upstream and downstream regulatory sequences.

The terms “level of expression” or “expression level” in general are used interchangeably and generally refer to the amount of a biomarker in a biological sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

The “presence,” “amount,” or “level” of a biomarker associated with an increased clinical benefit to a subject is a detectable level in a biological sample. These can be measured by methods known to one skilled in the art and also disclosed herein. The expression level or amount of biomarker assessed can be used to determine the response to the treatment.

“Reduced expression,” “reduced expression levels,” or “reduced levels” refers to a decrease expression or decreased levels of a biomarker in a subject relative to a control, such as a subject who is not suffering from the disease or disorder (e.g., asthma) or an internal control (e.g., housekeeping biomarker).

A “reference sample”, “reference cell”, “reference tissue”, “control sample”, “control cell”, or “control tissue”, as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject. In yet another embodiment, a reference sample, reference cell, reference tissue, control sample, control cell, or control tissue is obtained from a healthy and/or non-diseased part of the body (e.g., tissues or cells) of another subject. In even another embodiment, a reference sample, reference cell, reference tissue, control sample, control cell, or control tissue is obtained from an untreated tissue and/or cell of the body of another subject.

The term “sample,” as used herein, refers to a formulation that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

An “effective amount” of an agent, e.g., a pharmaceutical formulation, refers to an amount effective, at dosages and for periods of time necessary, to achieve the desired therapeutic result.

A “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In some embodiments, the subject is a human.

The term “patient” as used herein, refers to an animal, such as a mammal. In one embodiment, patient refers to a human.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “Th2-high asthma” as used herein, refers to asthma that exhibits high levels of one or more Th2 cell-related cytokines, for example, IL13, IL4, IL9, IL5, or that exhibits Th2 cytokine-associated inflammation. In some embodiments, the term Th2-high asthma may be used interchangeably with eosinophil-high asthma. In some embodiments, the Th2-high asthma is Th2 driven asthma. In some embodiments, the asthma patient has been determined to be Eosinophilic Inflammation Positive (EIP). See, e.g., International Patent Application Publication No. WO 2015/061441, which is incorporated by reference herein in its entirety. In some embodiments, the subject has been determined to have elevated levels of at least one of the eosinophilic signature genes as compared to a control or reference level. See WO2015/061441. In some embodiments, the Th2-high asthma is periostin-high asthma. In some embodiments, the subject has high serum periostin. In some embodiments, the subject is eighteen years or older. In some embodiments, the subject has been determined to have an elevated level of serum periostin as compared to a control or reference level. In some embodiments, the control or reference level is the median level of periostin in a population. In some embodiments, the subject has been determined to have 20 ng/ml or higher serum periostin. In some embodiments, the subject has been determined to have 25 ng/ml or higher serum periostin. In some embodiments, the subject has been determined to have 50 ng/ml or higher serum periostin. In some embodiments, the control or reference level of serum periostin is 20 ng/ml, 25 ng/ml, or 50 ng/ml. In some embodiments, the asthma is eosinophil-high asthma. In some embodiments, the subject has been determined to have an elevated eosinophil count as compared to a control or reference level. In some embodiments, the control or reference level is the median level of a population. In some embodiments, the subject has been determined to have 150 or higher eosinophil count/μl blood. In some embodiments, the subject has been determined to have 200 or higher eosinophil count/μl blood. In some embodiments, the subject has been determined to have 250 or higher eosinophil count/μl blood. In some embodiments, the subject has been determined to have 300 or higher eosinophil count/μl blood. In some embodiments, the subject has been determined to have 350 or higher eosinophil count/μl blood. In some embodiments, the subject has been determined to have 400 or higher eosinophil count/μl blood. In some embodiments, the subject has been determined to have 450 or higher eosinophil count/μl blood. In some embodiments, the subject has been determined to have 500 or higher eosinophil count/μl blood. In some preferred embodiments, the subject has been determined to have 300 or higher eosinophil count/μl blood. In some embodiments, the eosinophils are peripheral blood eosinophils. In some embodiments, the eosinophils are sputum eosinophils. In some embodiments, the subject exhibits elevated level of FeNO (fractional exhaled nitric acid) and/or elevated level of IgE. For example, in some instances, the subject exhibits a FeNO level above any of about 5 ppb (parts per billion), 10 ppb, 15 ppb, 20 ppb, 25 ppb, 30 ppb, 35 ppb, 40 ppb, 45 ppb, 50 ppb, 60 ppb, 70 ppb, 80 ppb, 90 ppb and 100 ppb. In some instances, the subject has an IgE level that is above 50 IU/ml.

The term “Th2-low asthma”, “non-Th2-high asthma”, “type 2-low asthma”, “T2-low asthma”, “non-eosinophilic asthma”, pauci-granulocytic asthma”, or “pauci-inflammatory asthma”, as used herein, refers to asthma that exhibits low levels of one or more Th2 cell-related cytokines, for example, IL13, IL4, IL9, IL5, or exhibits non-Th2 cytokine-associated inflammation. In some embodiments, the term Th2-low asthma may be used interchangeably with eosinophil-low asthma. In some embodiments, the asthma patient has been determined to be Eosinophilic Inflammation Negative (EIN). See, e.g., WO 2015/061441. In some embodiments, the Th2-low asthma is Th17-driven asthma. In some embodiments, the Th2-low asthma is periostin-low asthma. In some embodiments, the subject is eighteen years or older. In some embodiments, the subject has been determined to have a reduced level of serum periostin as compared to a control or reference level. In some embodiments, the control or reference level is the median level of periostin in a population. In some embodiments, the subject has been determined to have less than 20 ng/ml serum periostin. In some embodiments, the asthma is eosinophil-low asthma. In some embodiments, the subject has been determined to have a reduced eosinophil count as compared to a control or reference level. In some embodiments, the control or reference level is the medium level of a population. In some embodiments, the subject has been determined to have less than 150 eosinophil count/μl blood. In some embodiments, the subject has been determined to have less than 100 eosinophil count/μl blood. In certain preferred embodiments, the subject has been determined to have less than 300 eosinophil count/μl blood.

“Treatment” (and variations such as “treat” or “treating”) refers to clinical intervention in an attempt to alter the natural course of the subject or cell being treated. Desirable effects of treatment include one or more of preventing occurrence or recurrence of disease, alleviation of symptoms, diminishment of any direct or indirect pathological consequences of the disease, stabilized (i.e., not worsening) state of disease, decreasing the rate of disease progression, amelioration or palliation of the disease state, prolonging survival as compared to expected survival if not receiving treatment and improved prognosis.

The use of the terms “a” and “an” and “the” and similar terms in the context of describing embodiments herein are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. It is understood that aspects and embodiments provided herein include “consisting” and/or “consisting essentially of” aspects and embodiments.

As is understood by one skilled in the art, reference to “about” a value or parameter herein includes (and describes) embodiments that are directed to that value or parameter per se. For example, description referring to “about X” includes description of “X”.

The phrase “substantially different,” refers to a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values may be, for example, greater than about 10%, greater than about 20%, greater than about 30%, greater than about 40%, and/or greater than about 50% as a function of the value for the reference/comparator molecule.

The phrase “substantially similar,” as used herein, refers to a sufficiently high degree of similarity between two numeric values (generally one associated with a molecule and the other associated with a reference/comparator molecule) such that one of skill in the art would consider the difference between the two values to not be of statistical significance within the context of the biological characteristic measured by said values (e.g., Kd values). The difference between said two values may be, for example, less than about 20%, less than about 10%, and/or less than about 5% as a function of the reference/comparator value. The phrase “substantially normal” refers to substantially similar to a reference (e.g., normal reference).

### II. Methods of Using KLK5 Antagonists

Provided herein are methods of using a KLK5 antagonist for the inhibition of KLK5. For example, provided herein are methods for treating asthma in a subject comprising administering an effective amount of a KLK5 antagonist to the subject. In some embodiments, the KLK5 antagonist inhibits the serine protease activity of KLK5. In some embodiments, the KLK5 antagonist is selected from the group consisting of an antibody (e.g. anti-KLK5 antibody), a binding polypeptide (e.g., KLK5 binding polypeptide such as SPINK Fc fusion polypeptide), a polynucleotide (e.g., KLK5 polynucleotide antagonists such as siRNA or CRISPR-RNA, including sgRNAs having a CRISPR-RNA and tracrRNA sequence) and small molecule (e.g., KLK5 small molecule antagonists such as small molecule protease inhibitors). In some embodiments, the KLK5 antagonist is an antibody (e.g., a monoclonal antibody).

Further provided herein are methods of predicting the response of a subject suffering from asthma to a treatment comprising a KLK5 antagonist, the method comprising (a) measuring the KLK5 level in a biological sample from the subject, (b) comparing the KLK5 level detected in the sample to a reference level, and (c) predicting that the subject will respond to the treatment when the KLK5 level measured in the sample is elevated compared to the reference level and predicting that the subject will not respond to the treatment when the KLK level measured in the sample is reduced compared to the reference level. In some embodiments, the KLK5 antagonist inhibits the serine protease activity of KLK5. In some embodiments, the KLK5 antagonist is selected from the group consisting of an antibody (e.g., anti-KLK5 antibody), a binding polypeptide (e.g., KLK5 binding polypeptide such as SPINK Fc fusion polypeptide), a polynucleotide (e.g., KLK5 polynucleotide antagonists such as siRNA or CRISPR-RNA, including sgRNAs having a CRISPR-RNA and tracrRNA sequence), including and small molecule (e.g., KLK5 small molecule antagonists such as small molecule protease inhibitors). In some embodiments, the KLK5 antagonist is an antibody (e.g., a monoclonal antibody).

Further provided herein are methods of selecting a subject suffering from asthma for a treatment comprising a KLK5 antagonist, comprising determining the presence or absence of a genetic variation located in the KLK5 genomic sequence in a biological sample from the subject, wherein the presence of the genetic variation indicates that the subject is suitable for treatment with a KLK5 antagonist. In some embodiments, the KLK5 antagonist inhibits the serine protease activity of KLK5. In some embodiments, the KLK5 antagonist is selected from the group consisting of an antibody (e.g., anti-KLK5 antibody), a binding polypeptide (e.g., KLK5 binding polypeptide such as SPINK Fc fusion polypeptide), a polynucleotide (e.g., KLK5 polynucleotide antagonists such as siRNA or CRISPR-RNA, including sgRNAs having a CRISPR-RNA and tracrRNA sequence), and small molecule (e.g., KLK5 small molecule antagonists such as small molecule protease inhibitors). In some embodiments, the KLK5 antagonist is an antibody (e.g., a monoclonal antibody).

Further provided herein are methods for detecting the presence or absence of a genetic variation in the KLK5 genomic sequence indicating that a subject suffering from asthma is suitable for treatment with a KLK5 antagonist, comprising (a) contacting a sample from the subject with a reagent capable of detecting the presence or absence of the genetic variation located in the KLK5 genomic sequence; and (b) determining the presence or absence of the genetic variation, wherein the presence of the genetic variation indicates that the subject is suitable for treatment with a KLK5 antagonist. In some embodiments, the KLK5 antagonist inhibits the serine protease activity of KLK5. In some embodiments, the KLK5 antagonist is selected from the group consisting of an antibody (e.g., anti-KLK5 antibody), a binding polypeptide (e.g., KLK5 binding polypeptide such as SPINK Fc fusion polypeptide), a polynucleotide (e.g., KLK5 polynucleotide antagonists such as siRNA or CRISPR-RNA, including sgRNAs having a CRISPR-RNA and tracrRNA sequence), and small molecule (e.g., KLK5 small molecule antagonists such as small molecule protease inhibitors). In some embodiments, the KLK5 antagonist is an antibody (e.g., a monoclonal antibody). In some embodiments, the reagent is selected from an oligonucleotide, a DNA probe, an RNA probe, and a ribozyme. In some embodiments, the reagent is labeled.

Further provided herein are methods for selecting a compound for treating a disease associated with KLK5, comprising determining whether a test compound is a KLK5 antagonist, wherein a test compound that is a KLK5 antagonist is suitable as a compound for treating the disease associated with KLK5. In some embodiments, the KLK5 antagonist inhibits the serine protease activity of KLK5. In some embodiments, the KLK5 antagonist is selected from the group consisting of an antibody (e.g., anti-KLK5 antibody), a binding polypeptide (e.g., KLK5 binding polypeptide such as SPINK Fc fusion polypeptide), a polynucleotide (e.g., KLK5 polynucleotide antagonists such as siRNA or CRISPR-RNA, including sgRNAs having a CRISPR-RNA and tracrRNA sequence), and small molecule (e.g., KLK5 small molecule antagonists such as small molecule protease inhibitors). In some embodiments, the KLK5 antagonist is an antibody (e.g., a monoclonal antibody).

In some embodiments of any of the methods, the asthma is associated with elevated levels of KLK5 in a sample from the subject. In some embodiments, the asthma is associated with reduced activity of SPINK5 in a sample from the subject. In some embodiments, the asthma is associated with elevated levels of neutrophils in a sample from the subject. In some embodiments, the asthma is selected from the group consisting of type 2 low asthma, periostin low asthma and eosinophil low asthma. In some embodiments, the asthma is not associated with Netherton Syndrome. In some embodiments, the asthma is not associated with one or more genetic variations in the gene encoding SPINK5 or a gene product thereof. In some embodiments, the asthma is related to a genetic variation located in the KLK5 genomic sequence. In some embodiments, the method further comprises treating the subject for asthma based on the presence of the genetic variation. In some embodiments, the genetic variation is a SNP. In some embodiments, the genetic variation is SNP rs117639512.

In some embodiments of any of the methods, the asthma is persistent chronic severe asthma with acute events of worsening symptoms (exacerbations or flares) that can be life threatening. In some embodiments, the asthma is atopic (also known as allergic) asthma, non-allergic asthma (e.g., often triggered by infection with a respiratory virus (e.g., influenza, parainfluenza, rhinovirus, human metapneurnovirus, and respiratory syncytial virus) or inhaled irritant (air pollutants, smog, diesel particles, volatile chemicals and gases indoors or outdoors, or even by cold dry air). In some embodiments, the asthma is intermittent or exercise-induced, asthma due to acute or chronic primary or second-hand exposure to “smoke” (typically cigarettes, cigars, pipes), inhaling or “vaping” (tobacco, marijuana or other such substances), or asthma triggered by recent ingestion of aspirin or related NSAIDS. In some embodiments, the asthma is mild, or corticosteroid naïve asthma, newly diagnosed and untreated asthma, or not previously requiring chronic use of inhaled topical or systemic steroids to control the symptoms (cough, wheeze, shortness of breath/breathlessness, or chest pain). In some embodiments, the asthma is chronic, corticosteroid resistant asthma, corticosteroid refractory asthma, asthma uncontrolled on corticosteroids or other chronic asthma controller medications. In some embodiments, the asthma is moderate to severe asthma. In some embodiments, the asthma is Th2-high asthma. In some embodiments, the asthma is severe asthma. In some embodiments, the asthma is atopic asthma, allergic asthma, non-allergic asthma (e.g., due to infection and/or respiratory syncytial virus (RSV)), exercise-induced asthma, aspirin sensitive/exacerbated asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids. In some embodiments, the asthma is T helper lymphocyte type 2 (Th2) or type 2 (Th2) high, or Type 2 (T2)-driven asthma. In some embodiments, the asthma is eosinophilic asthma. In some embodiments, the asthma is allergic asthma. In some embodiments, the subject has been determined to be Eosinophilic Inflammation Positive (EIP). See WO2015/061441. In some embodiments, the asthma is periostin-high asthma (e.g., having periostin level at least about any of 20 ng/mL, 25 ng/mL, or 50 ng/mL serum). In some embodiments, the asthma is eosinophil-high asthma (e.g., at least about any of 150, 200, 250, 300, 350, 400 eosinophil counts/ml blood). In some embodiments, the asthma is Th2-low asthma or nonTh2-driven asthma. In some embodiments, the subject has been determined to be Eosinophilic Inflammation Negative (EIN). See WO2015/061441. In some embodiments, the asthma is periostin-low asthma (e.g., having periostin level less than about 20 ng/mL serum). In some embodiments, the asthma is eosinophil-low asthma (e.g., less than about 150 eosinophil counts/μl blood or less than about 100 eosinophil counts/μl blood).

In some embodiments of any of the methods, the sample is selected from the group consisting of cerebrospinal fluid, blood, serum, sputum, saliva, mucosal scraping, tissue biopsy, lacrimal secretion, semen, and sweat. In some embodiments, the sample is selected from the group consisting of bronchial alveolar lavage, lung parenchyma and bronchial sub-epithelium.

Presence and/or expression levels/amount of a biomarker can be determined qualitatively and/or quantitatively based on any suitable criterion known in the art, including but not limited to DNA, mRNA, cDNA, polypeptides, polypeptide fragments and/or gene copy number. In some embodiments, presence and/or expression levels/amount of a biomarker in a first sample is increased as compared to presence/absence and/or expression levels/amount in a second sample. In some embodiments, presence/absence and/or expression levels/amount of a biomarker in a first sample is decreased as compared to presence and/or expression levels/amount in a second sample. In some embodiments, the second sample is a reference sample, reference cell, reference tissue, control sample, control cell, or control tissue. Additional disclosures for determining presence/absence and/or expression levels/amount of a gene are described herein. In some embodiments KLK5 can be used as the biomarker. In some embodiments SPINK5 can be used as the biomarker.

In some embodiments of any of the methods, the KLK5 antagonist is administered to a subject in combination with an additional therapeutic agent. In some embodiments, the additional therapeutic agent is an IL-13 axis binding antagonist, an IL-5 axis binding antagonist, an IL-33 axis binding antagonist, an Ml prime antagonist, an IgE antagonist, a TRPA1 antagonist, a CRTH2 antagonist, a bronchodilator or asthma symptom controller medication, an immunomodulator, a corticosteroid, a Th2 pathway inhibitor, a tyrosine kinase inhibitor, or a phosphodiesterase inhibitor. In some embodiments, the IL-13 axis binding antagonist is an anti-IL-13 antibody. In some embodiments, the anti-IL-13 antibody is lebrikizumab. In some embodiments, the IL-5 axis binding antagonist is an IL-5 binding antagonist or an IL-5 receptor binding antagonist. In some embodiments, the IL-33 axis binding antagonist is an IL-33 binding antagonist or an ST2 binding antagonist. In some embodiments, the IL-33 binding antagonist is an anti-IL-33 antibody. In some embodiments, the Ml prime antagonist is quilizumab.

In some embodiments of any of the methods, the KLK5 antagonist is for administration subcutaneously, intravenously, intramuscularly, topically, orally, transdermally, intraperitoneally, intraorbitally, by implantation, by inhalation, intrathecally, intraventricularly, or intranasally. In some embodiments, the KLK5 antagonist is for administration subcutaneously. In some embodiments, the KLK5 antagonist is for use in a human subject.

In some embodiments of any of the methods, elevated expression refers to an overall increase of about any of 10%, 20%, 30%, 40%, 50%, 60%, 70%, 80%, 90%, 95%, 96%, 97%, 98%, 99% or greater, in the level of biomarker (e.g., polypeptide or nucleic acid (e.g., gene or mRNA)), detected by standard art known methods such as those described herein, as compared to a reference sample, reference cell, reference tissue, control sample, control cell, or control tissue. In some embodiments, the elevated expression refers to the increase in expression level/amount of a biomarker in the sample wherein the increase is at least about any of 1.5×, 1.75×, 2×, 3×, 4×, 5×, 6×, 7×, 8×, 9×, 10×, 25×, 50×, 75×, or 100× the expression level/amount of the respective biomarker in a reference sample, reference cell, reference tissue, control sample, control cell, or control tissue. In some embodiments, elevated expression refers to an overall increase of greater than about 1.5 fold, about 1.75 fold, about 2 fold, about 2.25 fold, about 2.5 fold, about 2.75 fold, about 3.0 fold, or about 3.25 fold as compared to a reference sample, reference cell, reference tissue, control sample, control cell, control tissue, or internal control (e.g., housekeeping gene). In some embodiments, the biomarker is a molecule involved in the KLK5 pathway. In some embodiments, the molecule is SPINK5. In some embodiments, the molecule is KLK5. In some embodiments, the molecule is a biological substrate of KLK5. In some embodiments, the biological substrate is selected from the group consisting of KLK7, KLK8, KLK14, PAR2 and an integrin/tissue matrix protein.

In some embodiments of any of the methods, reduced expression refers to an overall reduction of about any of 10%, 20%, 30%, 40%, 50%, 60%, 70%, 80%, 90%, 95%, 96%, 97%, 98%, 99% or greater, in the level of biomarker (e.g., polypeptide or nucleic acid (e.g., gene or mRNA)), detected by standard art known methods such as those described herein, as compared to a reference sample, reference cell, reference tissue, control sample, control cell, or control tissue. In some embodiments, reduced expression refers to the decrease in expression level/amount of a biomarker in the sample wherein the decrease is at least about any of 0.9×, 0.8×, 0.7×, 0.6×, 0.5×, 0.4×, 0.3×, 0.2×, 0.1×, 0.05×, or 0.01× the expression level/amount of the respective biomarker in a reference sample, reference cell, reference tissue, control sample, control cell, or control tissue.

Presence and/or expression level/amount of various biomarkers in a sample can be analyzed by a number of methodologies, many of which are known in the art and understood by the skilled artisan, including, but not limited to, immunohistochemical (“IHC”), Western blot analysis, immunoprecipitation, molecular binding assays, ELISA, ELIFA, fluorescence activated cell sorting (“FACS”), MassARRAY, proteomics, quantitative blood based assays (as for example Serum ELISA), biochemical enzymatic activity assays, in situ hybridization, Southern analysis, Northern analysis, whole genome sequencing, polymerase chain reaction (“PCR”) including quantitative real time PCR (“qRT-PCR”) and other amplification type detection methods, such as, for example, branched DNA, SISBA, TMA and the like), RNA-Seq, FISH, microarray analysis, gene expression profiling, and/or serial analysis of gene expression (“SAGE”), as well as any one of the wide variety of assays that can be performed by polypeptide, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

### III. KLK5 Antagonists

Provided herein are KLK5 antagonists for use in any of the methods described herein, e.g., methods of treating or diagnosing asthma or Netherton Syndrome. In some embodiments, the KLK5 antagonist is selected from the group consisting of an antibody (e.g., anti-KLK5 antibody), a binding polypeptide (e.g., KLK5 binding polypeptide such as SPINK Fc fusion polypeptide), a polynucleotide (e.g., KLK5 polynucleotide antagonists such as siRNA or CRISPR-RNA, including sgRNAs having a CRISPR-RNA and tracrRNA sequence), and small molecule (e.g., KLK5 small molecule antagonists such as small molecule protease inhibitors). In some embodiments, the antibody is a monoclonal antibody. In some embodiments, the antibody is a human, humanized, or chimeric antibody. In some embodiments, the antibody is a full length IgG1 antibody. A detailed description of KLK5 antagonists can be found in sections A.-E. herein below.

For example, the KLK5 antagonist according to any of the above embodiments binds to one or more residues of any of the amino acid sequences selected from the group consisting of SEQ ID NO:1, SEQ ID NO:2, SEQ ID NO:3, SEQ ID NO:4, SEQ ID NO:5, SEQ ID NO:6, SEQ ID NO:7, and SEQ ID NO:8. In some embodiments of any of the KLK5 antagonists, the KLK5 antagonist binds to any of the amino acid sequences selected from the group consisting of SEQ ID NO:1, SEQ ID NO:2, SEQ ID NO:3, SEQ ID NO:4, SEQ ID NO:5, SEQ ID NO:6, SEQ ID NO:7, and SEQ ID NO:8. In some embodiments, the KLK5 antagonist binds to one or more residues of the amino acid sequence SEQ ID NO:1 (amino acid residues 1-293 of UniProt No. Q9Y337). In some embodiments, the KLK5 antagonist binds to amino acid sequence SEQ ID NO:1 (amino acid residues 1-293 of UniProt No. Q9Y337). In some embodiments, the KLK5 antagonist binds to a specific binding region on KLK5. In some embodiments, the binding region is located within the active site of KLK5. In some embodiments, the binding region comprises about any of 1, 2, 3, 4, 5, 6, 7, 8, 9, and/or 10 amino acid residues of KLK5. In some embodiments, the binding region comprising one or more of the amino acid residues of KLK5 selected from the group consisting of the amino acid residues at position 108, 147, 150, 153, 168 and 245 of full-length unprocessed KLK5, i.e., including the signal peptide.

In some embodiments, the binding region comprises amino acid residues that are within about any of 10, 9, 8, 7, 6, 5, 4, 3, 2, and/or 1 angstroms (Å) of any atom of a KLK5 antagonist. In some embodiments, the binding region comprises amino acid residues that are within less than any of 10, 9, 8, 7, 6, 5, 4, 3, 2, and/or 1 Å of any atom of a KLK5 antagonist. In some embodiments, the binding region comprises amino acid residues that are within between any of 10-9, 9-8, 8-7, 7-6, 6-5, 5-4, 4-3, 3-2, and/or 2-1 Å of any atom of a KLK5 antagonist. In some embodiments, the binding region comprises amino acid residues that are within about any of 9.5 Å, 9 Å, 8.5 Å, 8 Å, 7.5 Å, 7 Å, 6.5 Å, 6 Å, 5.5 Å, 5 Å, 4.5 Å, 4 Å, 3.5 Å, 3 Å, 2.5 Å, 2 Å, 1.5 Å, and/or 1 Å of any atom of a KLK5 antagonist. The amino acid residues of a KLK5 antagonist that contact the binding region (i.e., paratope) can be determined, for example, by determining the crystal structure of the KLK5 antagonist in complex with the binding region or by performing hydrogen/deuterium exchange.

Further, the KLK5 antagonist according to any of the above embodiments substantially or completely inhibits the biological activity of KLK5. In some embodiments, the biological activity of KLK5 is serine protease activity. In some embodiments, the biological activity of KLK5 is tryptic-like serine protease activity. In some embodiments, the biological activity of KLK5 is KLK5 promoted human smooth muscle cell proliferation and contraction. In some embodiments, the biological activity of KLK5 is KLK5 induced epithelial expression of inflammatory cytokines, chemokines, and adhesion molecules. In some embodiments, the biological activity of KLK5 is KLK5 induced epithelium production of neutrophil chemotactic cytokines and neutrophil influx into the lung tissues. In some embodiments, the biological activity of KLK5 is inhibited by at least about any of 20%, 30%, 40%, 50%, 60%, 70%, 80%, 90% and/or more. In some embodiments, the biological activity of the KLK5 is inhibited by about any of 20%, 30%, 40%, 50%, 60%, 70%, 80%, 90% and/or more. In some embodiments, the biological activity of the KLK5 is inhibited by between any of 20-30%, 30-40%, 40-50%, 50-60%, 60-70%, 70-80%, 80-90%, and/or 90-100%.

In some embodiments of any of the KLK5 antagonists, the KLK5 antagonist substantially or completely inhibits binding of SPINK5 to KLK5. In some embodiments, binding of SPINK5 to KLK5 is inhibited by at least about any of 20%, 30%, 40%, 50%, 60%, 70%, 80%, 90% and/or more. In some embodiments, binding of SPINK5 to KLK5 is inhibited by about any of 20%, 30%, 40%, 50%, 60%, 70%, 80%, 90% and/or more. In some embodiments, binding of SPINK5 to KLK5 is inhibited by between any of 20-30%, 30-40%, 40-50%, 50-60%, 60-70%, 70-80%, 80-90%, and/or 90-100%.

In some embodiments of any of the KLK5 antagonists, a KLK5 antagonist has a binding affinity (dissociation constant) to KLK5 of less than about any of 10⁻⁷ nM, 10⁻⁸ nM, 10⁻⁹ nM, 10⁻¹⁰ nM, 10⁻¹¹ nM, 10⁻¹² nM, and/or 10⁻¹³ nM. In some embodiments, a KLK5 antagonist has a binding affinity to KLK5 of less than any of 10⁻⁷ nM, 10⁻⁸ nM, 10⁻⁹ nM, 10⁻¹⁰ nM, 10⁻¹¹ nM, 10⁻¹² nM, and/or 10⁻¹³ nM.

In some embodiments of any of the KLK5 antagonists, the KLK5 antagonist has an IC₅₀ of less than about any of 1000 nM, 500 nM, 100 nM, 50 nM, 10 nM, 5 nM, 1 nM, 500 pM, 100 pM, 50 pM, 10 pM, 5 pM, and/or 1 pM. In some embodiments, the KLK5 antagonist has an IC₅₀ of less than any of 1000 nM, 500 nM, 100 nM, 50 nM, 10 nM, 5 nM, 1 nM, 500 pM, 100 pM, 50 pM, 10 pM, 5 pM, and/or 1 pM. In some embodiments, the KLK5 antagonist has an IC₅₀ of between about any of 50 μM-1 μM, 1 μM-500 nM, 500 nM-100 nM, 100 nM-10 nM, 10 nM-1 nM, 1000 pM-500 pM, 500 pM-200 pM, 200 pM-150 pM, 150 pM-100 pM, 100 pM-10 pM, and/or 10 pM-1 pM.

A. Antibodies

Provided herein are isolated anti-KLK5 antibodies for use in the methods described herein. In any of the above embodiments, the anti-KLK5 antibody is humanized. Further, the anti-KLK5 antibody according to any of the above embodiments is a monoclonal antibody, including a chimeric, humanized or human antibody. In some embodiments, the anti-KLK5 antibody is an antibody fragment, e.g., a Fv, Fab, Fab′, scFv, diabody, or F(ab′)₂ fragment. In some embodiments, the anti-KLK5 antibody is a full length IgG1 antibody. In some embodiments, the anti-KLK5 antibody is a monoclonal mouse IgG2B antibody. In some embodiments, the monoclonal mouse IgG2B antibody is mAb1108 (Clone #193318, R & D Systems, Minneapolis, Minn.).

In a further aspect, the anti-KLK5 antibody according to any of the above embodiments may incorporate any of the features, singly or in combination, as described in Sections below:

1. Affinity

In some embodiments, the anti-KLK5 antibody provided herein has a dissociation constant (Kd) of ≤1 μM, ≤100 nM, ≤10 nM, ≤1 nM, ≤0.1 nM, ≤0.01 nM, and/or ≤0.001 nM (e.g., 10⁻⁸ M or less, e.g., from 10⁻⁸ M to 10⁻¹³ M, e.g., from 10⁻⁹ M to 10⁻¹³ M). In one embodiment, Kd is measured by a radiolabeled antigen binding assay (RIA). In one embodiment, the RIA is performed with the Fab version of an anti-KLK5 antibody and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Fisher Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CMS chips at ˜10 response units (RU). In one embodiment, carboxymethylated dextran biosensor chips (CMS, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled polypeptide. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{d}) is calculated as the ratio k_{off}/kₒₙ. See, e.g., Chen et al., J. Mol. Biol. 293:865-881 (1999). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrometer (Aviv Instruments) or a 8000-series SLM-AMINCO™ spectrophotometer (ThermoSpectronic) with a stirred cuvette.

2. Antibody Fragments

In some embodiments, the anti-KLK5 antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In some embodiments, a single-domain antibody is a human single-domain antibody (Domantis, Inc., Waltham, Mass.; see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

3. Chimeric and Humanized Antibodies

In some embodiments, the anti-KLK5 antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed. See e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity-determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In some embodiments, the anti-KLK5 antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMab® technology; U.S. Pat. No. 7,041,870 describing K-M MOUSE® technology, and U.S. Patent Application Publication No. US 2007/0061900, describing VelociMouse® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Hist. & Histopath., 20(3):927-937 (2005) and Vollmers and Brandlein, Methods Find Exp. Clin. Pharmacol., 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Anti-KLK5 antibodies may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. Methods Mol. Biol. 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, Methods Mol. Biol. 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In some embodiments, the anti-KLK5 antibody provided herein is a multispecific antibody, e.g., a bispecific antibody. Multispecific antibodies are monoclonal antibodies that have binding specificities for at least two different sites. In some embodiments, one of the binding specificities is KLK5 and the other is for any other antigen. In some embodiments, bispecific antibodies may bind to two different epitopes of KLK5. Bispecific antibodies may also be used to localize cytotoxic agents to cells which express KLK5. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multispecific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576A1).

The antibody or fragment herein also includes a “Dual Acting FAb” or “DAF” comprising an antigen binding site that binds to a polypeptide of interest, such as KLK5 as well as another, different antigen (see, US 2008/0069820, for example).

B. KLK5 Binding Polypeptides

Binding polypeptides which bind KLK5 (KLK5 binding polypeptides) are also provided for use in the methods described herein. In some embodiments, the KLK5 binding polypeptide is a KLK5 antagonist. In some embodiments, the KLK5 binding polypeptide is a fusion polypeptide. In some embodiments, the fusion polypeptide is a SPINK fusion polypeptide. In some embodiments, the SPINK fusion polypeptide is a SPINK Fc fusion polypeptide. In some embodiments, the SPINK Fc fusion polypeptide comprises 2 SPINK polypeptides or fragments thereof. In some embodiments of any of the binding polypeptides, each of the 2 SPINK polypeptides or fragments thereof comprises one or more domains of SPINK5. In some embodiments, each of the 2 SPINK5 polypeptides or fragments thereof comprises 1, 2, 3, 4, 5, 6, 7 and/or 8 Kazal domains. In some embodiments, each of the 2 SPINK5 polypeptides or fragments thereof comprises 1 Kazal domain (i.e., 2 Kazal domains per SPINK5 Fc fusion polypeptide). In some embodiments, each of the 2 SPINK5 polypeptides or fragments thereof comprises 4 Kazal domains (i.e., 8 Kazal domains per SPINK5 Fc fusion polypeptide). In some embodiments, the 4 Kazal domains are Kazal domains 6, 7, 8 and/or 9. In some embodiments, Kazal domains 6, 7, 8 and/or 9 are from mouse SPINK5 (UNIPROT Q5K5D4). In some embodiments, Kazal domains 6, 7, 8 and/or 9 comprise the amino acid residues E421-A695 from mouse SPINK5 (UNIPROT Q5K5D4). In some embodiments, the SPINK5 Fc fusion polypeptide comprises the SPINK5 amino acid sequence SEQ ID NO:17. In some embodiments, the Fc region of the SPINK5 Fc fusion polypeptide is selected from the group consisting of an IgG1 Fc region, IgG2a Fc region and IgG4 Fc region. In some embodiments, the Fc region is an IgG2a Fc region. In some embodiments, the IgG2a Fc region is a mouse IgG2a Fc region. In some embodiments, the SPINK5 Fc fusion polypeptide comprises the amino acid sequence SEQ ID NO:16. In some embodiments, each of the 2 SPINK5 polypeptides or fragments thereof comprises 1 Kazal domain (i.e., 2 Kazal domains per SPINK5 Fc fusion polypeptide). In some embodiments, the 1 Kazal domain is Kazal domain 4. In some embodiments, Kazal domain 4 is from mouse SPINK5 (UNIPROT Q5K5D4). In some embodiments, Kazal domain 4 comprises the amino acid residues M293-R355 from mouse SPINK5 (UNIPROT Q5K5D4). In some embodiments, the Fc region of the SPINK5 Fc fusion polypeptide is selected from the group consisting of an IgG1 Fc region, IgG2a Fc region and IgG4 Fc region. In some embodiments, the Fc region is an IgG2a Fc region. In some embodiments, the IgG2a Fc region is a mouse IgG2a Fc region. In some embodiments, the SPINK5 Fc fusion polypeptide comprises the SPINK5 amino acid sequence SEQ ID NO:22. In some embodiments, the SPINK5 Fc fusion polypeptide comprises the amino acid sequence SEQ ID NO:21. In some embodiments, the 4 Kazal domains are Kazal domains 8, 9, 10 and/or 11. In some embodiments, Kazal domains 8, 9, 10 and/or 11 are from human SPINK5 (UNIPROT Q9NQ38). In some embodiments, Kazal domains 8, 9, 10 and/or 11 comprise the amino acid residues E490-Y757 from human SPINK5 (UNIPROT Q9NQ38). In some embodiments, the SPINK5 Fc fusion polypeptide comprises the SPINK5 amino acid sequence SEQ ID NO:15. In some embodiments, the Fc region of the SPINK5 Fc fusion polypeptide is selected from the group consisting of an IgG1 Fc region, IgG2a Fc region and IgG4 Fc region. In some embodiments, the Fc region is an IgG1 Fc region. In some embodiments, the IgG1 Fc region is a human IgG1 Fc region. In some embodiments, the human IgG1 Fc region has the amino acid E at position 356. In some embodiments, the human IgG1 Fc region has the amino acid M at position 358. In some embodiments, the SPINK5 Fc fusion polypeptide comprises the amino acid sequence SEQ ID NO:13. In some embodiments, the Fc region is an IgG4 Fc region. In some embodiments, the IgG4 Fc region is a human IgG4 Fc region. In some embodiments, the human IgG4 Fc region has the amino acid S at position 228. In some embodiments, the human IgG4 Fc region has the amino acid P at position 228. In some embodiments, the SPINK5 Fc fusion polypeptide comprises the amino acid sequence SEQ ID NO:14. In some embodiments, each of the 2 SPINK5 polypeptides or fragments thereof comprises 1 Kazal domain (i.e., 2 Kazal domains per SPINK5 Fc fusion polypeptide). In some embodiments, the 1 Kazal domain is Kazal domain 5. In some embodiments, Kazal domain 5 is from human SPINK5 (UNIPROT Q9NQ38). In some embodiments, Kazal domain 5 comprises the amino acid residues R291-R352 from human SPINK5 (UNIPROT Q9NQ38). In some embodiments, the Fc region of the SPINK5 Fc fusion polypeptide is selected from the group consisting of an IgG1 Fc region, IgG2a Fc region and IgG4 Fc region. In some embodiments, the Fc region is an IgG1 Fc region. In some embodiments, the IgG1 Fc region is a human IgG1 Fc region. In some embodiments, the human IgG1 Fc region has the amino acid E at position 356. In some embodiments, the human IgG1 Fc region has the amino acid M at position 358. In some embodiments, the SPINK5 Fc fusion polypeptide comprises the SPINK5 amino acid sequence SEQ ID NO:20. In some embodiments, the SPINK5 Fc fusion polypeptide comprises the amino acid sequence SEQ ID NO:18. In some embodiments, the Fc region is an IgG4 Fc region. In some embodiments, the IgG4 Fc region is a human IgG4 Fc region. In some embodiments, the human IgG4 Fc region has the amino acid S at position 228. In some embodiments, the human IgG4 Fc region has the amino acid P at position 228. In some embodiments, the SPINK5 Fc fusion polypeptide comprises the amino acid sequence SEQ ID NO:19.

In some embodiments of any of the binding polypeptides, each of the 2 SPINK polypeptides or fragments thereof comprises 1 domain of SPINK9. In some embodiments, each of the 2 SPINK9 polypeptides or fragments thereof comprises 1 Kazal domain (i.e., 2 Kazal domains per SPINK9 Fc fusion polypeptide). In some embodiments, the 1 Kazal domain is Kazal domain 1. In some embodiments, Kazal domain 1 is from human SPINK9 (UNIPROT Q5DT21). In some embodiments, Kazal domain 1 comprises the amino acid residues I20-C86 from human SPINK9 (UNIPROT Q5DT21). In some embodiments, I20-C86 from human SPINK9 comprises the amino acid C at position 22. In some embodiments, I20-C86 from human SPINK9 comprises the amino acid S at position 22. In some embodiments, I20-C86 from human SPINK9 comprises the amino acid H at position 48. In some embodiments, I20-C86 from human SPINK9 comprises the amino acid R at position 48. In some embodiments, I20-C86 from human SPINK9 comprises the amino acid M at position 49. In some embodiments, I20-C86 from human SPINK9 comprises the amino acid E at position 49. In some embodiments, I20-C86 from human SPINK9 comprises the SPINK9 amino acid sequence SEQ ID NO:28. In some embodiments, the human Fc region of the SPINK9 Fc fusion polypeptide is selected from the group consisting of an IgG1 Fc region, IgG2a Fc region and IgG4 Fc region. In some embodiments, the Fc region is an IgG1 Fc region. In some embodiments, the IgG1 Fc region is a human IgG1 Fc region. In some embodiments, the human IgG1 Fc region has the amino acid E at position 356. In some embodiments, the human IgG1 Fc region has the amino acid M at position 358. In some embodiments, the SPINK9 Fc fusion polypeptide comprises the amino acid sequence SEQ ID NO:25. In some embodiments, the Fc region is an IgG2a Fc region. In some embodiments, the IgG2a Fc region is a human IgG2a Fc region. In some embodiments, the SPINK9 Fc fusion polypeptide comprises the amino acid sequence SEQ ID NO:27. In some embodiments, the Fc region is an IgG4 Fc region. In some embodiments, the IgG4 Fc region is a human IgG4 Fc region. In some embodiments, the human IgG4 Fc region has the amino acid S at position 228. In some embodiments, the human IgG4 Fc region has the amino acid P at position 228. In some embodiments, the SPINK9 Fc fusion polypeptide comprises the amino acid sequence SEQ ID NO:26.

KLK5 binding polypeptides may be chemically synthesized using known polypeptide synthesis methodology or may be prepared and purified using recombinant technology. KLK5 binding polypeptides are usually at least about 5 amino acids in length, alternatively at least about 10, 15, 20, 25, 30, 35, 40, 45, 50, 55, 60, 65, 70, 75, 80, 85, 90, 95, and/or 100 amino acids in length and/or more, wherein such KLK5 binding polypeptides that are capable of binding, preferably specifically, to KLK5.

KLK5 binding polypeptides may be identified without undue experimentation using well known techniques. In this regard, it is noted that techniques for screening polypeptide libraries for binding polypeptides that are capable of specifically binding to KLK5 are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

Methods of generating peptide libraries and screening these libraries are also disclosed in U.S. Pat. Nos. 5,723,286, 5,432,018, 5,580,717, 5,427,908, 5,498,530, 5,770,434, 5,734,018, 5,698,426, 5,763,192, and 5,723,323.

C. KLK5 Small Molecule Antagonists

Provided herein are small molecules for use as a KLK5 small molecule antagonist for use in the methods described above. In some embodiments, the small molecule antagonist substantially or completely inhibits KLK5 biological activity. In some embodiments, the biological activity is a serine protease activity. In some embodiments, the biological activity is a tryptic-like serine protease activity. In some embodiments, the KLK5 small molecule antagonist is a protease inhibitor. In some embodiments, the protease inhibitor is leupeptin.

Small molecules are preferably organic molecules other than binding polypeptides or antibodies as defined herein that bind, preferably specifically, to KLK5 as described herein. Binding organic small molecules may be identified and chemically synthesized using known methodology (see, e.g., PCT Publication Nos. WO00/00823 and WO00/39585). Binding organic small molecules are usually less than about 2000 daltons in size, alternatively less than about 1500, 750, 500, 250 or 200 daltons in size, wherein such organic small molecules that are capable of binding, preferably specifically, to a polypeptide as described herein may be identified without undue experimentation using well known techniques. In this regard, it is noted that techniques for screening organic small molecule libraries for molecules that are capable of binding to a polypeptide of interest are well known in the art (see, e.g., PCT Publication Nos. WO00/00823 and WO00/39585). Binding organic small molecules may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

D. KLK5 Antagonist Polynucleotides

Provided herein are also KLK5 polynucleotide antagonists for use in the methods described herein. The KLK5 polynucleotide antagonist may be an antisense nucleic acid and/or a ribozyme. The antisense nucleic acids comprise a sequence complementary to at least a portion of an RNA transcript of KLK5. However, absolute complementarity, although preferred, is not required.

The KLK5 polynucleotide antagonist may be a nucleic acid that hybridizes under stringent conditions to KLK5 nucleic acid sequences (e.g., siRNA and CRISPR-RNA, including sgRNAs having a CRISPR-RNA and tracrRNA sequence). See Mali et al., Science. 339: 823-26, (2013).

A sequence “complementary to at least a portion of an RNA,” referred to herein, means a sequence having sufficient complementarity to be able to hybridize with the RNA, forming a stable duplex; in the case of double stranded antisense nucleic acids, a single strand of the duplex DNA may thus be tested, or triplex formation may be assayed. The ability to hybridize will depend on both the degree of complementarity and the length of the antisense nucleic acid. Generally, the larger the hybridizing nucleic acid, the more base mismatches with a RNA it may contain and still form a stable duplex (or triplex as the case may be). One skilled in the art can ascertain a tolerable degree of mismatch by use of standard procedures to determine the melting point of the hybridized complex.

Polynucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. See generally, Wagner, R., 1994, Nature 372:333-335. Thus, oligonucleotides complementary to either the 5′- or 3′-non-translated, non-coding regions of the gene, could be used in an antisense approach to inhibit translation of endogenous mRNA. Polynucleotides complementary to the 5′ untranslated region of the mRNA should include the complement of the AUG start codon. Antisense polynucleotides complementary to mRNA coding regions are less efficient inhibitors of translation. Whether designed to hybridize to the 5′-, 3′- or coding region of an mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

E. Variants of Antibodies and Binding Polypeptides Described Herein

1. Glycosylation Variants

In any of the above embodiments, the antibody (e.g., anti-KLK5 antibody) or the binding polypeptide (e.g., KLK5 binding polypeptide) provided herein is altered to increase or decrease the extent to which the antibody or the binding polypeptide is glycosylated. Addition or deletion of glycosylation sites a polypeptide may be conveniently accomplished by altering the amino acid sequence such that one or more glycosylation sites is created or removed.

Where the antibody or binding polypeptide comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the antibody or binding polypeptide as described herein may be made in order to create variants with certain improved properties.

In one embodiment, antibody or binding polypeptide variants are provided having a carbohydrate structure that lacks fucose attached (directly or indirectly) to an Fc region. For example, the amount of fucose in such antibody or Fc fusion polypeptide may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies or binding polypeptides. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al., Biotech. Bioeng. 87: 614 (2004). Examples of cell lines capable of producing defucosylated antibodies include Lec13 CHO cells deficient in polypeptide fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

2. Fc Region Variants

In some embodiments, one or more amino acid modifications may be introduced into the Fc region of the antibody (e.g., anti-KLK5 antibody) or the binding polypeptide (e.g., KLK5 binding polypeptide). The Fc region variant may comprise a human Fc region sequence (e.g., a human IgG1, IgG2, IgG3 or IgG4 Fc region) comprising an amino acid modification (e.g., a substitution) at one or more amino acid positions.

In some embodiments, provided is an antibody variant or binding polypeptide variant that possesses some but not all effector functions, which make it a desirable candidate for applications in which the half-life of the antibody or binding polypeptide in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody or binding polypeptide lacks FcγR binding (hence likely lacking ADCC activity), but retains FcRn binding ability. The primary cells for mediating ADCC, NK cells, express Fc(RIII) only, whereas monocytes express Fc(RI), Fc(RII) and Fc(RIII). FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody or binding polypeptide variants with improved or diminished binding to FcRs are described. (See, e.g., U.S. Pat. No. 6,737,056; WO 2004/056312, and Shields et al., J. Biol. Chem. 9(2): 6591-6604 (2001).) In some embodiments, an antibody variant or binding polypeptide variant comprises an Fc region with one or more amino acid substitutions which improve ADCC, e.g., substitutions at positions 298, 333, and/or 334 of the Fc region (EU numbering of residues). In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

3. Cysteine Engineered Variants

In some embodiments, it may be desirable to create cysteine engineered antibody (e.g., anti-KLK5 antibody) or the binding polypeptide (e.g., KLK5 binding polypeptide), in which one or more residues are substituted with cysteine residues. In particular embodiments, the substituted residues occur at accessible sites of the antibody or the binding polypeptide. By substituting those residues with cysteine, reactive thiol groups are thereby positioned at accessible sites of the antibody and may be used to conjugate the antibody or the binding polypeptide to other moieties, such as drug moieties or linker-drug moieties, to create an immunoconjugate, as described further herein. In some embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies or Fc fusion polypeptides may be generated as described, e.g., in U.S. Pat. No. 7,521,541.

4. Amino Acid Variants Antibody Variants

In some embodiments, amino acid sequence variants of the antibody (e.g., anti-KLK5 antibody) or the binding polypeptide (e.g., KLK5 binding polypeptide) provided herein are contemplated. For example, it may be desirable to improve the binding affinity and/or other biological properties of antibody or the binding polypeptide. Amino acid sequence variants of the antibody or the binding polypeptide may be prepared by introducing appropriate modifications into the nucleotide sequence encoding the antibody or the binding polypeptide, or by peptide synthesis. Such modifications include, for example, deletions from, and/or insertions into and/or substitutions of residues within the amino acid sequences of the antibody or the binding polypeptide. Any combination of deletion, insertion, and substitution can be made to arrive at the final construct, provided that the final construct possesses the desired characteristics, e.g., antigen-binding.

In some embodiments, the antibody variants or the binding polypeptide variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into the antibody or the binding polypeptide and the products screened for a desired activity, e.g., retained/improved antigen binding, decreased immunogenicity, or improved ADCC or CDC.

TABLE 1

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; | Ile |
| | Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

5. Derivatives

In some embodiments, the antibody (e.g., anti-KLK5 antibody) or the binding polypeptide (e.g., KLK5 binding polypeptide) provided herein can be further modified to contain additional nonproteinaceous moieties that are known in the art and readily available. The moieties suitable for derivatization of the antibody or the binding polypeptide include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody and/or binding polypeptide may vary, and if more than one polymer are attached, they can be the same or different molecules. In general, the number and/or type of polymers used for derivatization can be determined based on considerations including, but not limited to, the particular properties or functions of the antibody and/or binding polypeptide to be improved, whether the antibody derivative and/or binding polypeptide derivative will be used in a therapy under defined conditions, etc.

In another embodiment, conjugates of an antibody and/or binding polypeptide to nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody and/or binding polypeptide-nonproteinaceous moiety are killed.

### IV. Pharmaceutical Formulations and Methods of Administration

Pharmaceutical formulations of the KLK5 antagonists as described herein are prepared by mixing such antagonists having the desired degree of purity with one or more optional pharmaceutically acceptable carriers in the form of lyophilized formulations or aqueous solutions. See Remington's Pharmaceutical Sciences 16th edition, Osol, A. Ed. (1980). In some embodiments, the KLK5 antagonists provided herein are antibodies (e.g., anti-KLK5 antibodies), binding polypeptides (e.g., KLK5 binding polypeptide), polynucleotides (e.g., KLK5 polynucleotide antagonists such as siRNA or CRISPR-RNA, including sgRNAs having a CRISPR-RNA and tracrRNA sequence), and small molecules (e.g., small molecule protease inhibitor).

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. See Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the KLK5 antagonist which matrices are in the form of shaped articles, e.g., films, or microcapsules.

The formulations to be used for in vivo administration are generally sterile. Sterility may be readily accomplished, e.g., by filtration through sterile filtration membranes.

Further provided herein are pharmaceutical formulations comprising a KLK5 antagonist for use in the methods described herein. In some embodiments, the formulation comprises a pharmaceutically acceptable carrier, adjuvant, or vehicle. In some embodiments, the formulation comprises an amount of the compound effective to measurably inhibit KLK5 protease activity. In some embodiments, the formulation is formulated for administration to a subject in need thereof.

Formulations comprising a KLK5 antagonist may be administered orally, parenterally, by inhalation spray, topically, transdermally, rectally, nasally, buccally, sublingually, vaginally, intraperitoneal, intrapulmonary, intradermal, epidural or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

Specific dosage and treatment regimen for any particular subject will depend upon a variety of factors, including age, body weight, general health, sex, diet, time of administration, rate of excretion, drug combination, the judgment of the treating physician, and the severity of the particular disease being treated. The amount of a provided KLK5 antagonist in the formulation will also depend upon the particular compound in the formulation.

In one embodiment, the effective amount of the KLK5 antagonist administered per dose will be in the range of about 0.01-100 mg/kg, alternatively about 0.1 to 20 mg/kg of subject body weight per day, with the typical initial range of compound used being 0.3 to 15 mg/kg/day.

The KLK5 antagonist may be employed alone or in combination with other agents for treatment as described above. For example, the second agent of the pharmaceutical combination formulation or dosing regimen may have complementary activities to the KLK5 antagonist such that they do not adversely affect each other. The compounds may be administered together in a unitary pharmaceutical formulation or separately.

The term “co-administering” refers to either simultaneous administration, or any manner of separate sequential administration, of a KLK5 antagonist, and a further active pharmaceutical ingredient or ingredients. If the administration is not simultaneous, the compounds are administered in a close time proximity to each other. Furthermore, it does not matter if the compounds are administered in the same dosage form, e.g., one compound may be administered topically and another compound may be administered orally.

Typically, any agent that has activity against a disease or condition being treated may be co-administered. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the disease involved.

### V. Methods of Screening and/or Identifying KLK5 Antagonists with Desired Function

Additional KLK5 antagonists for use in the methods described herein, including antibodies (e.g., anti-KLK5 antibodies), binding polypeptides (e.g., KLK5 binding polypeptides), polynucleotides (e.g., KLK5 polynucleotide antagonists such as siRNA or CRISPR-RNA, including sgRNAs having a CRISPR-RNA and tracrRNA sequence), and small molecules (e.g., KLK5 small molecule antagonists such as small molecule protease inhibitors) may be identified, screened for, or characterized for their physical/chemical properties and/or biological activities by various assays known in the art.

A candidate KLK5 antagonist may be computationally evaluated and designed by means of a series of steps in which chemical entities or fragments are screened and selected for their ability to associate with individual binding target sites on KLK5. One skilled in the art may use one of several methods to screen chemical entities or fragments for their ability to associate with KLK5, and more particularly with target sites on KLK5. The process may begin by visual inspection of, for example a target site on a computer screen, based on the KLK5 coordinates, or a subset of those coordinates known in the art.

In some embodiments of any of the methods of screening and/or identifying, the candidate KLK5 antagonist is anti-KLK5 antibody, KLK5 binding polypeptide (e.g., SPINK5 Fc fusion polypeptide or SPINK9 Fc fusion polypeptide), KLK5 polynucleotide antagonist or KLK5 small molecule antagonist. In some embodiments, the KLK5 antagonist substantially or completely inhibits the biological activity of the KLK5. In some embodiments, the biological activity is serine protease activity. In some embodiments, the biological activity is tryptic-like serine protease activity. In some embodiments, the KLK5 antagonist binds to a specific binding region on KLK5. In some embodiments, the KLK5 antagonist binds to the active site of KLK5.

The anti-KLK5 antibodies, KLK5 binding polypeptides, KLK5 polynucleotide antagonists, and/or KLK5 small molecule antagonists provided herein may be identified, screened for, or characterized for their physical/chemical properties and/or biological activities by various assays known in the art.

In one aspect, the anti-KLK5 antibodies, KLK5 binding polypeptides, KLK5 polynucleotide antagonists, and/or KLK5 small molecule antagonists provided herein is tested for its KLK5 binding activity, e.g., by known methods such as ELISA, western blotting analysis, cell surface binding by Scatchard or surface plasmon resonance. In another aspect, competition assays may be used to identify an antibody that competes with the anti-KLK5 antibody or KLK5 binding polypeptide provided herein for binding to KLK5. In a further aspect, the anti-KLK5 antibody or KLK5 binding polypeptide provided herein can be used for detecting the presence or amount of KLK5 present in a biological sample. In some embodiments, the biological sample is first blocked with a non-specific isotype control antibody to saturate any Fc receptors in the sample.

In one aspect, assays are provided for identifying the biological activity of the anti-KLK5 antibody or KLK5 binding polypeptide provided herein. In some embodiments, such assays for identifying the biological activity are e.g., peptide substrate assays or coupled assays. Biological activity of the anti-KLK5 antibody or KLK5 binding polypeptide may include, e.g., binding to KLK5, and thereby reducing the biological activity of KLK5. In some embodiments, the biological activity of the anti-KLK5 antibody or KLK5 binding polypeptide may include binding to other species of KLK polypeptides (e.g., KLK7, KLK8 and KLK14) and thereby reducing their biological activity.

### VI. Articles of Manufacture

In another aspect, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a formulation which is by itself or combined with another formulation effective for treating, preventing and/or diagnosing the condition and may have a sterile access port (for example the container may be an intravenous solution bag or a vial having a stopper pierceable by a hypodermic injection needle). At least one active agent in the formulation is a KLK5 antagonist as described herein. The label or package insert indicates that the formulation is used for treating the condition of choice. Moreover, the article of manufacture may comprise (a) a first container with a formulation contained therein, wherein the formulation comprises a KLK5 antagonist and (b) a second container with a formulation contained therein, wherein the formulation comprises an asthma therapy agent.

In some embodiments, the article of manufacture comprises a container, a label on said container, and a formulation contained within said container; wherein the formulation includes one or more reagents (e.g., primary antibodies that bind to one or more biomarkers or probes and/or primers to one or more of the biomarkers described herein), the label on the container indicating that the formulation can be used to evaluate the presence of one or more biomarkers in a sample, and instructions for using the reagents for evaluating the presence of one or more biomarkers in a sample. The article of manufacture can further comprise a set of instructions and materials for preparing the sample and utilizing the reagents. In some embodiments, the article of manufacture may include reagents such as both a primary and secondary antibody, wherein the secondary antibody is conjugated to a label, e.g., an enzymatic label. In some embodiments, the article of manufacture one or more probes and/or primers to one or more of the biomarkers described herein.

In some embodiments of any of the article of manufacture, the KLK5 antagonist is an anti-KLK5 antibody, KLK5 binding polypeptide, KLK5 polynucleotide antagonists and/or KLK5 small molecule antagonist as provided herein.

The article of manufacture in this embodiment may further comprise a package insert indicating that the formulations can be used to treat a particular condition. In some embodiments, the package insert comprises instructions for administering the KLK5 antagonist as asthma therapy agent. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Other optional components in the article of manufacture include one or more buffers (e.g., block buffer, wash buffer, substrate buffer, etc.), other reagents such as substrate (e.g., chromogen) which is chemically altered by an enzymatic label, epitope retrieval solution, control samples (positive and/or negative controls), control slide(s) etc.

### EXAMPLES

The following are examples of methods and formulations. It is understood that various other embodiments may be practiced, given the general description provided above. The use of any and all examples, or exemplary language (e.g., “such as”) provided herein, is intended merely to better illuminate embodiments and does not necessarily impose any limitations unless otherwise specifically recited in the claims. All documents cited herein are incorporated by reference in their entirety.

### Example 1

### Material and Methods

All institutional studies were reviewed and approved by local institutional review boards. In addition, all subjects gave informed consent before genotyping. Genotyping was done on a variety of different platforms summarized in Table 2.

TABLE 2

| Dataset_Name | genome-wide_SNP_array_ID |
|---|---|
| A1 | unknown |
| A2 | unknown |
| A3 | HumanOmni25M-8v1-1_B.bpm |
| C1 | unknown |
| C2 | HumanOmni2.5M-8v1-1_B.bpm |
| C3 | unknown |
| E | HumanOmni2.5-8v1-Multi_A.bpm |
| B | HumanOmni2.5-8v1-Multi_A.bpm |
| MI | HumanOmni2.5-8v1-Multi_A.bpm |
| V | HumanOmni2.5-8v1-Multi_A.bpm |
| MO | HumanOmni2.5-8v1-Multi_A.bpm |
| L | HumanOmni2.5-8v1-Multi_A.bpm |
| CG | HumanHap550v3 |
| NY | HumanHap550v3 |

Sample QC was performed in this order (1) Call rate <95% (N=84 removed) (2) Heterozygosity (N=82 removed) (3) Relatedness/Duplicates/IBD (N=22 removed) (4) Ancestry outliers (N=262 removed). For each separate dataset, EIGENSTRAT analysis was ran with HapMap samples and samples were excluded if they were outliers with respect to the European (CEPH and TSI) group (N=383).

SNP QC was performed in that SNPs were excluded if they (1) had a call rate <95%, (2) were monomorphic and (3) strongly deviated from Hardy-Weinberg equilibrium (P<1×10⁻⁷). A liftover to hg19 was performed for datasets which were not aligned to that build. In addition, the imputation pipeline requires that all datasets be aligned to the plus since the HapMap data were on the plus strand. Shapeit was used to check for strand issues and flipped to the plus strand when able to. Lastly, SNPs in chr1-chr22 for imputation were selected. The merged discovery dataset had 299,784 SNPs overlapping the asthma case dataset and the population based dataset. There were 230,853 SNPs overlapping the 8 different case and control datasets that make up the replication dataset.

Genome-wide imputation was performed using HapMap reference haplotypes and genotype data passing quality control as inference. Post imputation genotypic probabilities were used in a logistic regression model in SNPTESTv2. In addition, the discovery dataset was adjusted for population stratification and the replication dataset by adjusting for significant principal components; PCs were selected that explain >1% of the variance (see below). SNPs with an imputation info <0.6 were excluded from analysis. Additional post-analysis QC include the removal of any SNPs with a MAF<2% in the controls and SNPs that had a HWE p-value <1E⁻¹⁰ in the cases and controls combined. PLINK was then used to run meta-analysis on the discovery and replication results. A heterogeneity p-value cutoff of 0.1 was used to determine whether a fixed effects or random effects model should be used for the meta-analysis.

GTEx data used in this analysis were obtained from the online GTEx Portal (http://www.gtexportal.org/home/testyourown). The search was conducted on Nov. 11, 2016; the commands entered for KLK5 were:

rs117639512,KLK5,Esophagus_Gastroesophageal_Junction;
rs117639512,KLK5,Esophagus_Muscularis;
rs117639512,KLK5,Skin_Not_Sun_Exposed_Suprapubic;
rs117639512,KLK5,Skin_Sun_Exposed_Lower_leg. The commands entered for KLK4 were:
rs117639512,KLK4,Prostate; rs117639512,KLK4,Uterus.

Binding affinities of SPINK9 to KLK5 were measured by Surface Plasmon Resonance (SRP) using a BIAcore™-T200 instrument. SPINK9 with a murine IgG2a fragment crystallizable region (Fc) expressed in-house were captured by Protein A biosensor chip (GE Healthcare, cat#29127557) to achieve approximately 100 response units (RU). For kinetics measurements, four-fold serial dilutions (200 nM to 0.1953 nM) of human KLK5 binding polypeptide were the injected in HBS-T buffer at 25° C. with a flow rate of 30 μl/min. Association rates (kₒₙ) and dissociation rates (k_{off}) were calculated using a simple one-to-one Langmuir binding model (BIAcore Evaluation T200 Software version 2.0). The equilibrium dissociation constant (K_{D}) was calculated as the ratio k_{off}/kₒₙ.

### Subjects Used in Meta-Analysis

A total of 1,350 adult asthmatics and 3,690 controls were used in the meta-analysis. Of these, after quality control measures, 667 asthmatics were in the type 2 low asthma (called periostin-low) group, and 626 in the type 2 inflammatory (called periostin-high) group. The average age of cases was 45 years (SD=15) and 41 years (SD=15) for controls. All subjects were of European Caucasian descent. The majority of subjects (57.8%) were female. Average FEV1% predicted was 72.9 (SD=17) in cases and 101.6 (SD=8) in controls. Cases and controls were divided into two cohorts. Cohort 1 included asthmatic DNA samples obtained from Genentech observational and clinical trials for lebrikizumab (anti IL-13) and Xolair (anti IgE) (Total N=520). Cohort 2 included a completely independent set of DNA samples obtained from Genentech clinical trials for lebrikizumab (N=234) and from adult asthma patients ascertained at the Queensland Institute for Medical Research (N=774) and the University of Chicago (N=226). Samples were compared to controls selected based on genetically determined ancestry (Cohort 1; N=3,120) and screened by a pulmonologist (Cohort 2; N=1,146) to be negative for asthma. All cases, and controls from Cohort 2 were assayed for serum periostin levels, and the median protein level was used to separate the subjects into periostin-low and periostin-high subgroups. Characteristics of each cohort were shown in Table 3. The table only includes samples that passed the QC and were included in the analysis.

TABLE 3

| | Cohort 1 | | | Cohort 2 | | | |
|---|---|---|---|---|---|---|---|
| | Cases | | Controls | Cases | | Controls | |
| N | 468 | | 2808 | 882 | | 882 | |
| Age, | 43.95 | (13.1) | * | 46.08 | (15.2) | 40.20 | (14.9) |
| mean (SD) | | | | | | | |
| Sex, N (%) | 299 | (63.9%) | 1437 (n | 585 | (66.3%) | 580 | (65.7%) |
| female | | | n47.9%) | | | | |
| FEV1% | 70.8 | (13.4) | — | 75.1 | (20.4) | 101.6 | (7.7) |
| predicted, | | | | | | | |
| mean (SD) | | | | | | | |

*) Age data is in ranges 69 ≤ 54 yrs, 489 = 55-59 yrs, 761 = 60-64 yrs, 810 = 65-69 yrs, 503 = 70-74 yrs, 153 ≥ 75 yrs.

### Known Asthma Risk Allele Analysis

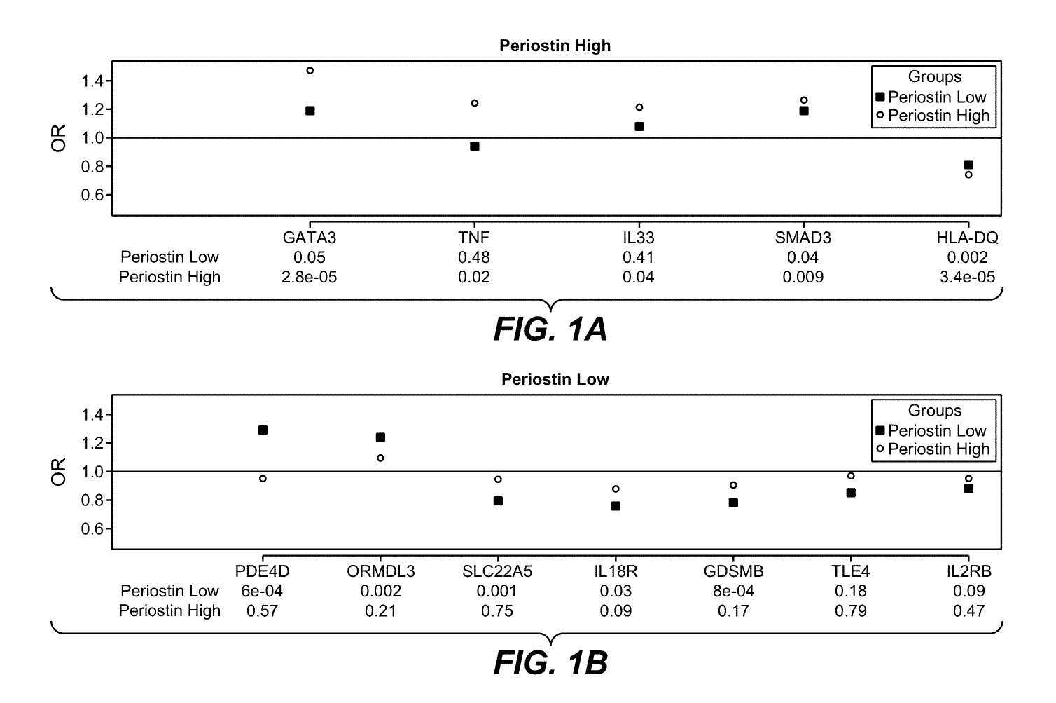

Currently, the extent of the genetic heterogeneity between type 2 inflammation and type 2 low asthmatics is unknown. The study population was stratified based on periostin levels, as described. See Corren et al., N Engl J Med 365, 1088-1098 (2011). Allele frequencies for the known asthma risk alleles was first compared between the periostin-high cases (N=626) and controls (N=1,696), and periostin-low cases (N=667) and controls (N=1,887). Enrichment in the effect size compared to controls for the periostin high and periostin low subgroups was determined. Results are shown in FIG. 1 and Table 4. Several of the known asthma genes (e.g., TSLP, IL4, IL4R, IL6R) showed no differences between subgroups. The odds ratios (OR) of several Th2 associated loci (e.g., GATA3 and IL33) were enriched in the periostin high subgroup. The PDE4D locus showed essentially a null OR in the periostin high subgroup (OR=0.96) and a strong enrichment in the periostin low subgroup (P=6.0×10⁻⁴; OR=1.3). This was the only locus to show a statistically different allele frequency between periostin low and high cases directly (P=0.02). Thus, many of the published asthma loci that were observed were pan-asthma loci, which is intuitive given that these studies did not differentiate subjects based on type 2 inflammation status. However, other loci differed between the subgroups, suggesting that novel loci will be revealed when dividing the asthma population by periostin status. The periostin low asthma subgroup was focused upon given the aforementioned dearth of knowledge and predicted unmet medical need around this patient population.

### Periostin Low Asthma Vs Controls GWAS

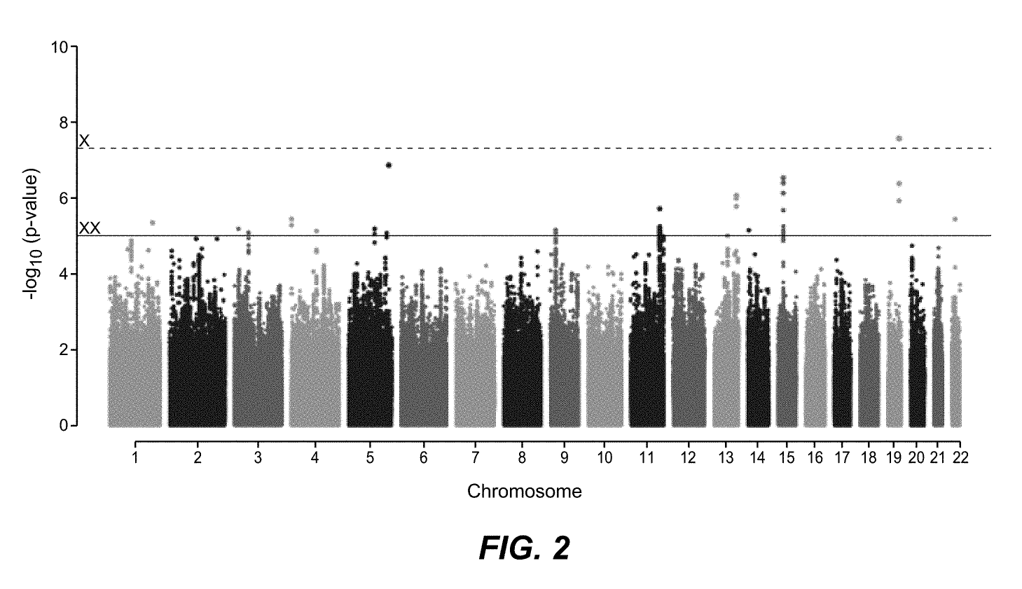

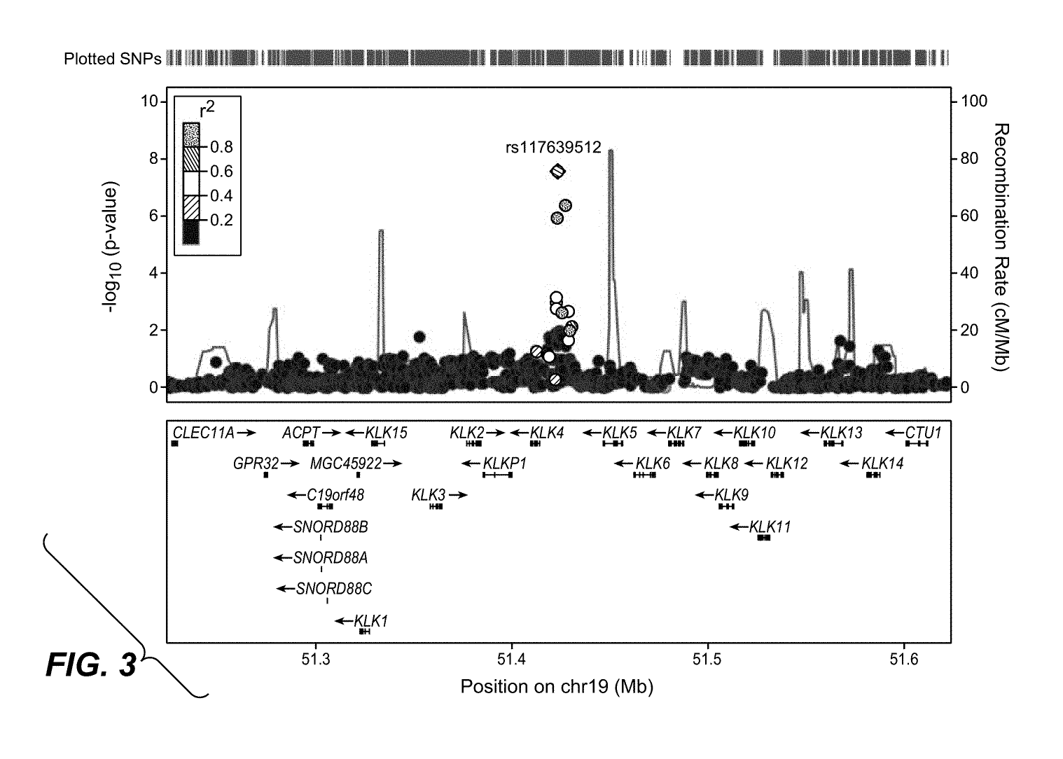

Using the healthy controls with serum periostin level measurement (N=790), a GWAS using periostin as a continuous trait was performed, and found no loci reaching genome-wide significance. This suggested that periostin level in normal controls was not under strong genetic influence. Therefore the entire control population in a periostin low asthma (N=667) vs controls (N=1,887) GWAS was used. All interesting SNPs reaching genome-wide significance in this analysis for association with periostin level in controls were tested in order to determine that the SNP(s) were associated specifically with type 2 low inflammation asthma, and not simply with the level of peripheral periostin. In total, one association for SNP rs117639512 was observed to exceed the threshold for genome-wide significance (P=2.75×10⁻⁸, OR=0.33, FIG. 2). The full list of SNPs with a P<1×10⁻⁵ (LD pruned) is shown in Table 5. Detailed information for SNP rs117639512 is shown in Table 6. The rs117639512 SNP was not associated with peripheral periostin level in the subset of controls with periostin measurement (P=0.99). In addition, the population was also stratified for eosinophil (EOS) level (level for EOS low <300 ng/mL) to see if the association in the periostin low asthmatics was also seen in EOS low asthmatics. Both were indicators of type 2 activity but were not perfectly correlated (ρ=0.23). See Anon et al., Ann Am Thorac Soc 10 Suppl, S206-213 (2013). SNP rs117639512 was tested for association in the asthma cases with low EOS (N=390) compared to controls (N=1,768) finding a similar direction of effect as seen in the periostin low analysis (P=0.008; OR=0.51). SNP rs117639512 was located in a large kallikrein (KLK) gene cluster locus containing 11 KLKs within a 500 kb stretch of DNA (FIG. 3). This SNP was located in the KLK5 genomic sequence. The association appeared to be specific for type 2 low asthma as the P-value was not significant in type 2 inflammation high patients (Table 6: Detailed association analysis for rs117639512, P=0.63, OR=1.11).

KLK5 is a Candidate Gene at this Locus

In order to identify the relevant gene in this locus, mRNA expression patterns were first examined. Using publicly available databases, KLK4 was predominantly expressed in prostate and endometrium while KLK5 was predominantly expressed in esophagus and skin. See Wu et al., Nucleic Acids Res 44, D313-316 (2016). The GTEx portal database (See Consortium, Science 348, 648-660 (2015)) was queried to investigate a possible functional effect of rs117639512 on KLK4 and KLK5 mRNA levels in the predominantly expressed tissues. The effect of rs117639512 on KLK4 could not be assessed in prostate or uterus as it was monomorphic in both tissues in the GTEx database. GTEx contains four tissues total for esophagus and skin (esophagus—gastroesophageal junction and muscularis; skin—sun exposed and not sun exposed). A KLK5 eQTL did not reach statistical significance in any of these tissues (lowest P=0.051 in sun exposed skin), at least in part due to the low minor allele frequency of the SNP (0.01-0.05 in European Caucasian populations, See Genomes Project, Auton et al., Nature 526, 68-74 (2015)). A global reference for human genetic variation. All tissues except esophagus—GE junction showed a lower mRNA level for KLK5 in the heterozygotes compared to the major allele homozygotes. There were no minor allele homozygotes in the database for comparison. Thus, it appears the minor allele of rs117639512 was linked to lower KLK5 mRNA levels, however, due to the low minor allele frequency of the rs117639512, larger databases are needed to confirm this hypothesis. Of interest, Netherton syndrome is caused by mutations in the gene SPINK5. See Descargues et al., Nat Genet 37, 56-65 (2005). SPINK5 encodes LEKTI, which is a serine protease inhibitor of KLK5 and KLK7. See Schechter et al., Biol Chem 386, 1173-1184 (2005). The mutations in SPINK5 lead to highly upregulated KLK5 expression which in turn induces inflammation through PAR2 (protease-activated receptor 2) dependent and independent pathways. See Hovnanian, A., Cell Tissue Res 351, 289-300 (2013). While Netherton syndrome was most commonly associated with skin disorders, asthma is co-morbid in some cases. See Judge et al., Br J Dermatol 131, 615-621 (1994). Thus, based on linkage disequilibrium, expression patterns, and syndromic comorbidities, KLK5 is the most relevant candidate gene at this locus. Furthermore, the direction of effect from the eQTL analysis was consistent with the protective OR for this SNP, such that lower KLK5 levels appear protective from asthma risk.

### Assays for Determination of KLK5 Inhibition

A recombinant KLK5 direct activity assay was used to measure the inhibition of human kallikrein 5 (KLK5) by KLK5 inhibitors such as SPINK Fc fusion polypeptides and mAb1108. Recombinant human KLK5 (Genentech) was diluted to 5 nM in direct assay buffer (75 mM Tris (pH 8.0), 150 mM NaCl and 0.01% Tween 20) and combined with anti-KLK5 antibodies in 384-well assay plate (384 Well Low Volume, Black, Round Bottom, Corning, Catalog No. 4514). Antibodies were supplied in either phosphate sample buffer (70 mM sodium phosphate (pH 6), 200 mM NaCl and 0.01% Tween-20) or citrate/Tris sample buffer (10 mM citric acid, 30 mM Tris (pH 6) and 0.01% Tween 20). Antibody dilutions were made in the appropriate sample buffer or in direct assay buffer. Plates were incubated for 30 minutes at ambient temperature. Fluorescent peptide substrate, Boc-VPR-AMC (Bachem, Part No. 1-1120) was added directly to the assay plate. Final in-well concentrations were 50 μM Boc-VPR-AMC, 5 nM recombinant human KLK5, and 0.19-100 nM anti-KLK5 antibodies. Plates were examined every 102 s for 30-60 minute using a PHERAstar® Plus reader using a 340 nm excitation/460 nm emission module. The RFU/s reaction rate was calculated by linear regression of readings in the linear range, typically beginning at 204 s and continuing until the end of the assay. Buffer alone and 100 nM final SPINK9.SRE.Fc (Genentech) were used as 100% and 0% activity controls, respectively. The IC₅₀ of the anti-KLK5 antibodies were determined from a four-parameter fit for their respective curves.

A coupled pro-KLK7 fluorescent peptide assay was used to measure the inhibition of human kallikrein 5 (KLK5) by anti-KLK5 antibodies. Recombinant human KLK5 (Genentech) was diluted to 5 nM in pro-KLK7 citrate/Tris coupled buffer (50 mM Tris (pH 7.5), 150 mM NaCl and 0.01% Tween 20) if antibody samples were in citrate/Tris sample buffer or pro-KLK7 phosphate coupled buffer (50 mM Tris (pH 8.0), 150 mM NaCl and 0.01% Tween 20) if antibody samples were in phosphate sample buffer. Diluted KLK5 was then combined with anti-KLK5 antibodies in 384-well assay plate (384 Well Low Volume, Black, Round Bottom, Corning, Catalog No. 4514). Antibody dilutions were made as described for the direct KLK5 assay. Plates were incubated for 30 minutes at ambient temperature. Fluorescent peptide substrate, suc-LLVY-AMC (Bachem, Part No. 1-1395) and pro-KLK7 (Genentech) were added directly to the assay plate and incubated at ambient temperature. Final in-well concentrations were 100 μM suc-LLVY-AMC, 125 nM pro-KLK7, 5 nM recombinant human KLK5, and 0.19-100 nM anti-KLK5 antibodies. After 24 hours, fluorescent readings were made every 102 s for 30-60 min and the RFU endpoint value was calculated by averaging the last 5 readings. Buffer alone and 100 nM final SPINK9.SRE.Fc (Genentech) were used as 100% and 0% activity controls, respectively. The IC₅₀ of the anti-KLK5 antibodies were determined from a four-parameter fit for their respective curves.

A recombinant KLK7 fluorescent peptide assay was used to determine the selectivity of KLK5 inhibitors. Recombinant human KLK7 (Genentech) was activated with KLK5 in pro-KLK7 phosphate coupled buffer (50 mM Tris (pH 8.0), 150 mM NaCl and 0.01% Tween 20). Diluted KLK7 was then combined with KLK5 inhibitors in 384-well assay plate (384 Well Low Volume, Black, Round Bottom, Corning, Catalog No. 4514). Inhibitor dilutions were made as described for the direct KLK5 assay. Plates were incubated for 50 minutes at ambient temperature. Fluorescent peptide substrate, suc-LLVY-AMC (Bachem, Part No. 1-1395) and pro-KLK7 (Genentech) were added directly to the assay plate and incubated at ambient temperature. Final in-well concentrations were 100 μM suc-LLVY-AMC, 125 nM pro-KLK7, 5 nM recombinant human KLK5, and 0.19-100 nM KLK5 inhibitors. After 24 hours, fluorescent readings were made every 102 s for 30-60 min and the RFU endpoint value was calculated by averaging the last 5 readings. Buffer alone and 100 nM final SPINK9.SRE.Fc (Genentech) were used as 100% and 0% activity controls, respectively. The IC₅₀ of the KLK5 inhibitors were determined from a four-parameter fit for their respective curves.

A pro-KLK7 assay was performed using KLK5-derived cleavage peptide detection by LC/MS for IC₅₀ determination. To perform the pro-KLK7 assay, the product peptide EEAQGDK (SEQ ID NO:30) from reaction between the enzyme KLK5 and substrate pro-KLK7 was detected by mass spectrometry coupled to liquid chromatography. All compounds were diluted with 50 mM ammonium bicarbonate buffer (Powder/Certified, Fisher Chemical, A643-500) with final concentrations in the assay at 5 nM KLK5 (Genentech) and inhibitors ranging from 0.01 to 12 nM, diluted in 96-well plates (Biorad, Hard-Shell 96-Well PCR Plates, low profile, thin wall, skirted, blue/clear #HSP9631). Inhibitors used were SPINK9.SRE.Fc (Genentech) and mAb1108 (Monoclonal Mouse IgG2b Clone #193318, R & D Systems, Minneapolis, Minn.). Plates were incubated at room temperature for 30 minutes. Afterward, 15 nM of substrate pro-KLK7 (Genentech) was added to the enzyme plus inhibitors. After 2 hours, the reaction was quenched using 0.5 uL of Formic Acid (99.5+%, Optima™ LC/MS Grade, Fisher Chemical, A117-10X1AMP). Peptide was detected using the combination of the following masses: Q1, 388.7 m/z and Q3, 319.0 m/z, in a QTRAP 6500 LC-MS/MS mass spectrometer (Sciex, Framingham, Mass.). Quantitation of generated peptide was measured using a synthetic KLK7 peptide calibration curve. The IC₅₀ values were determined using Prism 6 Software (GraphPad Software, La Jolla, Calif.).

A pro-KLK1 assay was performed using KLK5-derived cleavage peptide detection by LC/MS for IC₅₀ determination. To perform the pro-KLK1 assay, the product peptide APPIQSR (SEQ ID NO:31) from reaction between the enzyme KLK5 and substrate pro-KLK1 was detected by mass spectrometry coupled to liquid chromatography. All compounds were diluted with 50 mM ammonium bicarbonate buffer (Powder/Certified, Fisher Chemical, A643-500) with final concentrations in the assay at 0.5 nM KLK5 (Genentech) and inhibitors ranging from 0.01 to 12 nM, diluted in 96-well plates (Biorad, Hard-Shell 96-Well PCR Plates, low profile, thin wall, skirted, blue/clear #HSP9631). Inhibitors used were SPINK9.SRE.Fc (Genentech) and mAb1108 (Monoclonal Mouse IgG2b Clone #193318, R & D Systems, Minneapolis, Minn.). Plates were incubated at room temperature for 60 minutes. Afterward, 300 nM of substrate pro-KLK1 (Genentech) was added to the enzyme plus inhibitors. After 20 minutes, the reaction was quenched using 0.5 uL of Formic Acid (99.5+%, Optima™ LC/MS Grade, Fisher Chemical, A117-10X1AMP). Peptide was detected using the combination of the following masses: Q1, 384.7 m/z and Q3, 600.3 m/z, in a QTRAP 6500 LC-MS/MS mass spectrometer (Sciex, Framingham, Mass.). IC₅₀ values were determined using peak areas and Prism 6 Software (GraphPad Software, La Jolla, Calif.).

### Example 2—Characterization of KLK5 in Asthma

### KLK5 was Expressed and Elevated in Asthmatic Lung Tissue

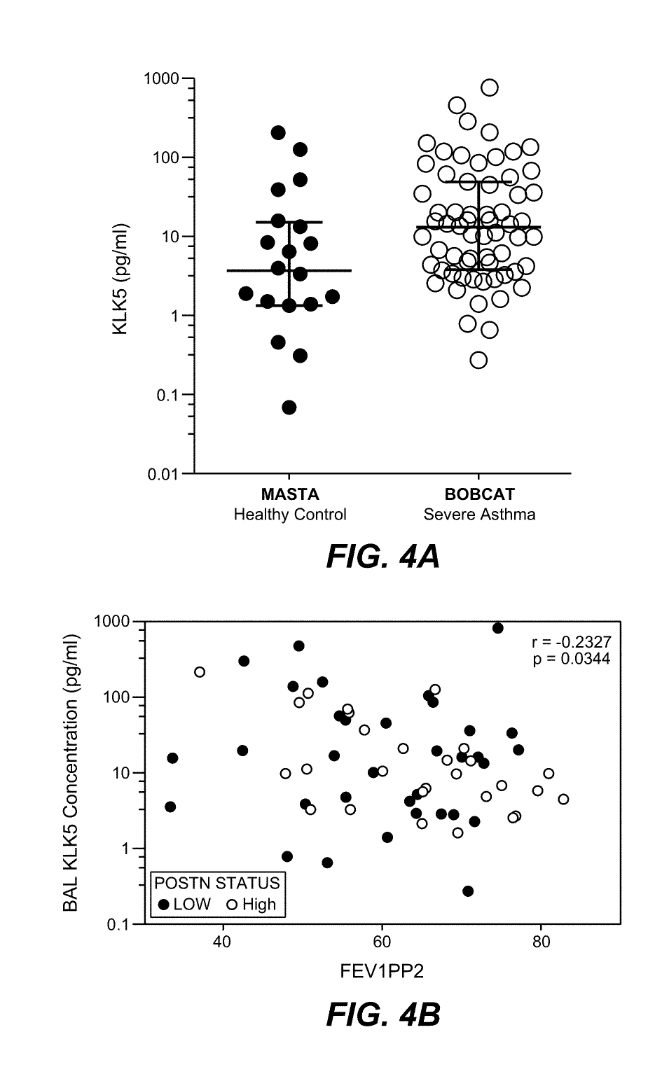

KLK5 expression in lung tissue was examined. A sensitive immune-assay was developed to measure KLK5 in bronchial alveolar lavage (BAL) of healthy donors (MAST-A cohort) and corticosteroid-refractory asthma patients (BOBCAT cohort). See Jia et al., J Allergy Clin Immunol 130, 647-654 e610 (2012) and Sun et al., Sci Signal 8, ra122 (2015). The average level of KLK5 was elevated about four-fold in asthma patients as compared to healthy volunteers (FIG. 4). In addition, the level of KLK5 in BAL of asthma patients was negatively correlated with predicted forced expiratory volume 1 (FEV1) (P<0.05), indicating that patients with increased KLK5 may have more severe bronchial obstruction and airway disease. Levels of KLK5 in lung were not associated with serum Th2 biomarkers (periostin and blood eosinophils), and both periostin-high and -low asthma patients had similar levels of BAL KLK5. In order to understand the cellular sources of KLK5, KLK5 transcript levels were compared in various primary lung resident cells. KLK5 mRNA was strongly expressed by bronchial epithelial cells, and was undetectable in lung smooth muscle, fibroblast, endothelial cells, or mononuclear cells. To examine KLK5 expression in situ, its expression was examined by using a KLK5-LacZ reporter mouse line with LacZ in the open reading frame of the KLK5 promoter. LacZ positive cells were largely restricted to bronchial epithelial cells. Taken together, these data suggest bronchial epithelial cells were likely the main cellular source for KLK5 in lung and contribute to the KLK5 in bronchial alveolar lavage.

### Recombinant KLK5 Induced Lung Neutrophil Extravasation & Lung Epithelium Cytokine Production

Next recombinant KLK5 was generated and its biochemical function was characterized. Recombinant full-length KLK5 with a C-terminal his tag was expressed in 293 cells. Secreted KLK5 had the pro-sequence (aa23-66) removed and started with N terminal isoleucine at position 67. A serine-to-alanine mutation at position 245 (S245A) abolished KLK5 catalytic activity and the S245A KLK5 mutant had an intact N terminal pro-sequence. The results suggest that auto-activation and signal peptide removal was likely self-intrinsic to KLK5.

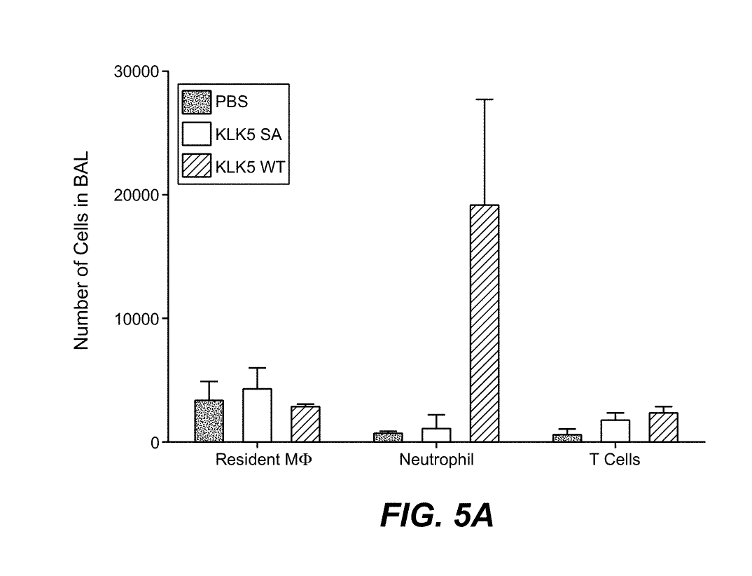

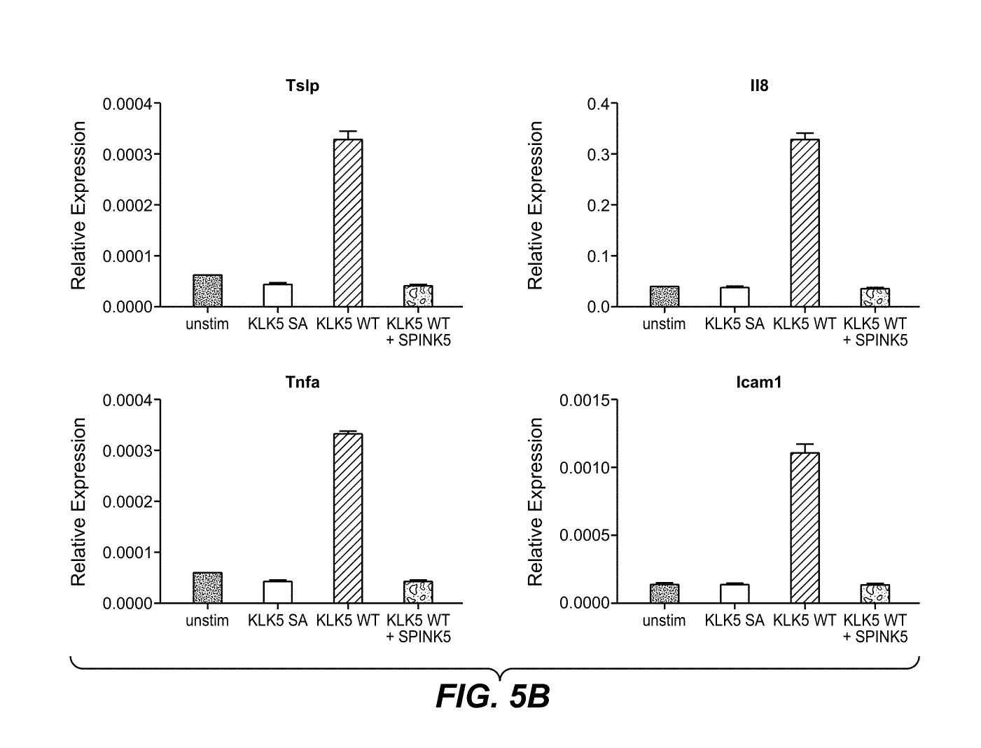

To investigate the effect of KLK5 in lung, recombinant KLK5 was administered intranasally to mice. Twenty-four hours after KLK5 administration, a greater than a 10-fold increase in the number of neutrophils in the bronchial alveolar lavage fluid was observed (FIG. 5A). There was no significant change in the number of eosinophils, macrophages, or lymphocytes. Selective recruitment of neutrophils was also increased in a tissue section of lung parenchyma and these granulocytes were localized to the bronchial sub-epithelium. Intranasal administration of catalytic mutant KLK5 did not result in neutrophil extravasation. Thus, the ability of KLK5 to recruit neutrophils into the lung compartment was highly dependent on the enzymatic activity of the protease.

To understand how KLK5 affects neutrophil recruitment, recombinant KLK5 was added into an A549 lung epithelial cell line and examined the expression of inflammation cytokines and chemokines through quantitative PCR. KLK5, but not its catalytically inactive mutant, rapidly induced pro-inflammation gene transcripts including Tslp, Tnfa, Il8, and Icam1 (FIG. 5B). The induction of Tslp, Tnfa, IL-8, and Icam1 was also seen with primary isolated bronchial epithelium cells. Furthermore, SPINK5 Fc fusion polypeptide inhibited KLK5-stimulated inflammation cytokine and chemokine production.

### Example 3—Inhibition of KLK5 in Direct Assay and Coupled Assay

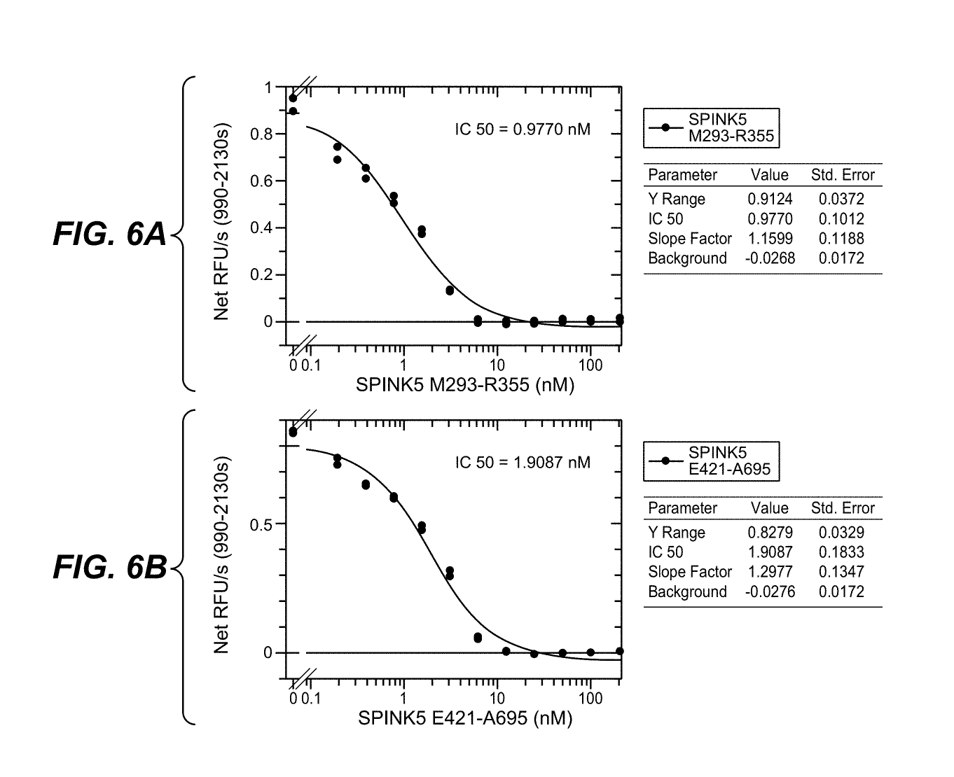

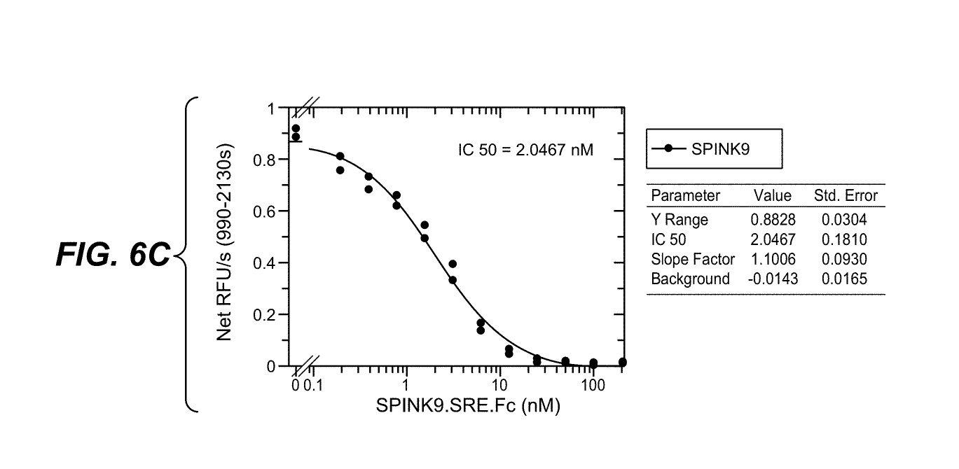

To evaluate the inhibitory profile of the SPINK Fc fusion polypeptides, an in vitro assay that monitors the cleavage of a fluorescent peptide substrate by KLK5 was developed. In brief, KLK5 cleaves the peptide bond between the terminal arginine of the substrate, Boc-VPR-AMC, releasing the 7-Amino-4-methylcoumarin (AMC) resulting in an increase in fluorescence. Incubation of KLK5 with SPINK5 M293-R355 (FIG. 6A), SPINK5 E421-A695 (FIG. 6B) or SPINK9.SRE.Fc (FIG. 6C) prior to addition of fluorescent substrate results in reduced fluorescent signal due to inactivation of KLK5. Thus, the SPINK Fc fusion polypeptides are potent inhibitors of KLK5 as monitored by Boc-VPR-AMC cleavage.

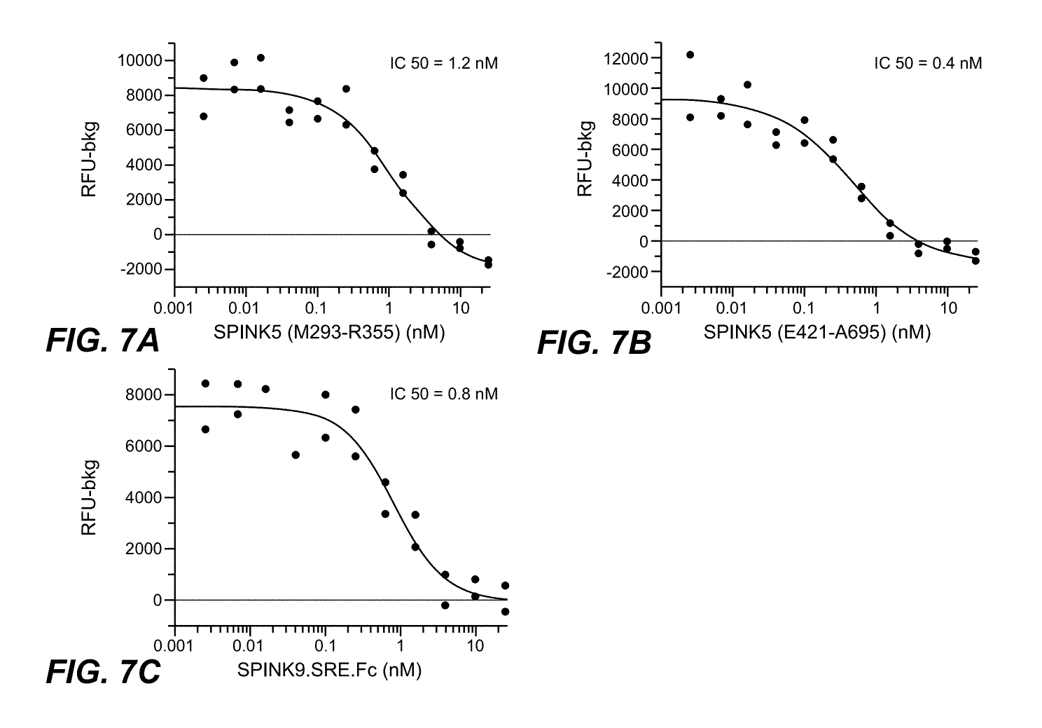

As demonstrated in FIG. 6, the SPINK Fc fusion polypeptides are potent inhibitors of KLK5 as monitored by the cleavage of a small peptide substrate. To further evaluate the inhibitory profile of these SPINK Fc fusion polypeptides, a coupled assay was developed utilizing pro-KLK7 and a specific KLK7 fluorescent peptide substrate, Suc-LLVY-AMC (FIG. 7). In short, KLK5 is incubated with pro-KLK7 resulting in cleavage and removal of the KLK7 pro-domain. The removal of the pro-domain activates KLK7 that is then able to act on the fluorescent substrate to release the AMC fluorophore. Similar to data using the small peptide substrate (FIG. 6), incubation of KLK5 with SPINK5 M293-R355 (FIG. 7A), SPINK5 E421-A695 (FIG. 7B) or SPINK9.SRE.Fc (FIG. 7C) resulted in potent inhibition of the activation of pro-KLK7 and subsequent cleavage of the KLK7 specific peptide substrate. Taken together, FIGS. 6 and 7 demonstrate that SPINK Fc fusion polypeptides are potent inhibitors of KLK5 using either a peptide or macromolecular (pro-KLK7) substrate.

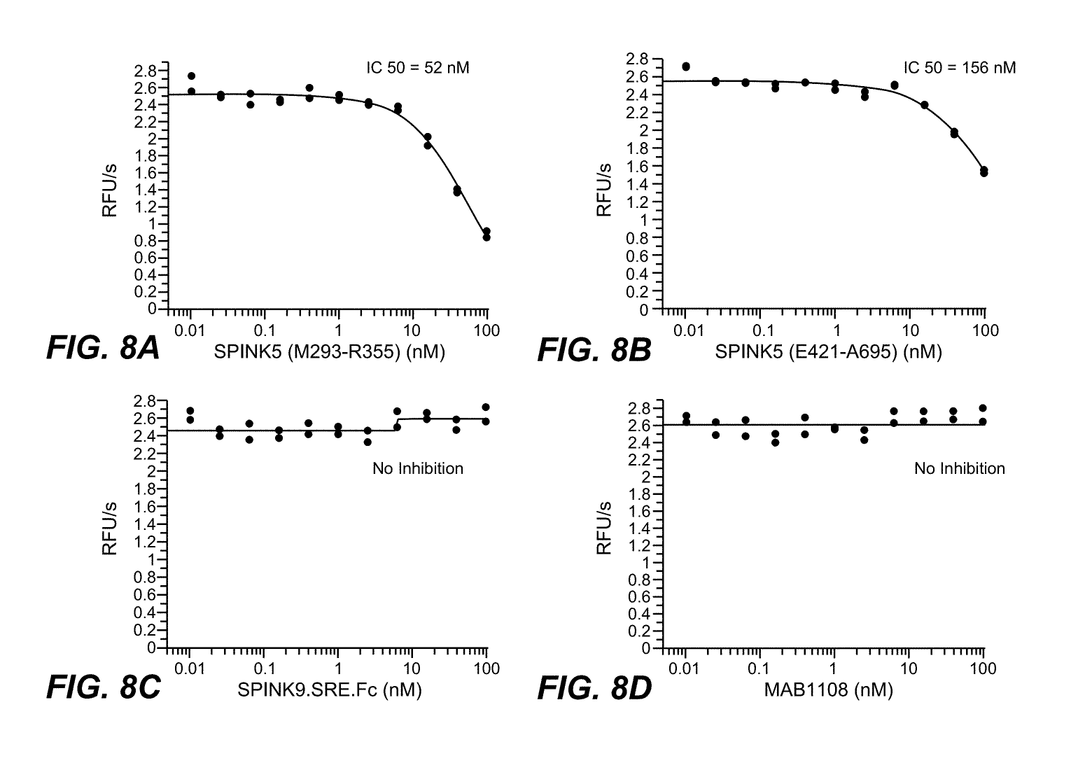

To evaluate the specificity of the SPINK Fc fusion polypeptides, the inhibitors were assayed against activated KLK7 and the cleavage of the fluorescent peptide substrate, Suc-LLVY-AMC, was monitored (FIG. 8). As a control for the KLK specificity a commercial anti-KLK5 antibody, mAb1108, was also assayed. As this antibody is specific to KLK5, it was anticipated that it should not inhibit KLK7 or the cleavage of the substrate. As seen in FIG. 8, both SPINK5 M293-R355 (FIG. 8A) and SPINK5 E421-A695 (FIG. 8B) partly inhibited KLK7, while SPINK9.SRE.Fc (FIG. 8C) and mAb1108 (FIG. 8D) demonstrated no inhibition. This indicated that SPINK9.SRE.Fc and mAb1108 specifically interact and inhibit KLK5 while SPINK5 M293-R355 and SPINK5 E421-A695 may be a promiscuous KLK inhibitor.

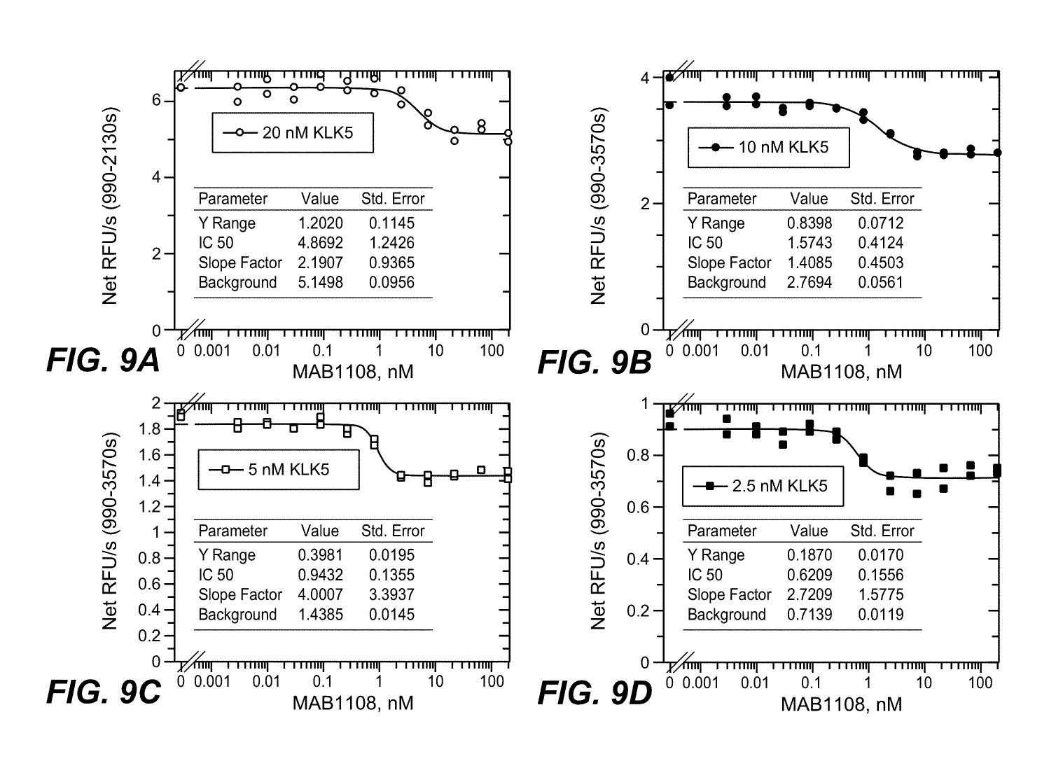

To characterize the inhibition profile of anti-KLK5 antibody, mAb1108, the IC₅₀ value in the direct assay (FIG. 6) at various KLK5 concentrations (FIG. 9) was determined. Unlike the SPINK Fc fusion polypeptides, mAb1108 is a partial inhibitor of KLK5 resulting in ˜30% reduction in cleavage of the fluorescent peptide substrate (FIG. 9). Additionally, the IC₅₀ values of mAb1108 demonstrate a dependence on KLK5 concentration, suggesting that antibody is likely a tight binding inhibitor of KLK5.

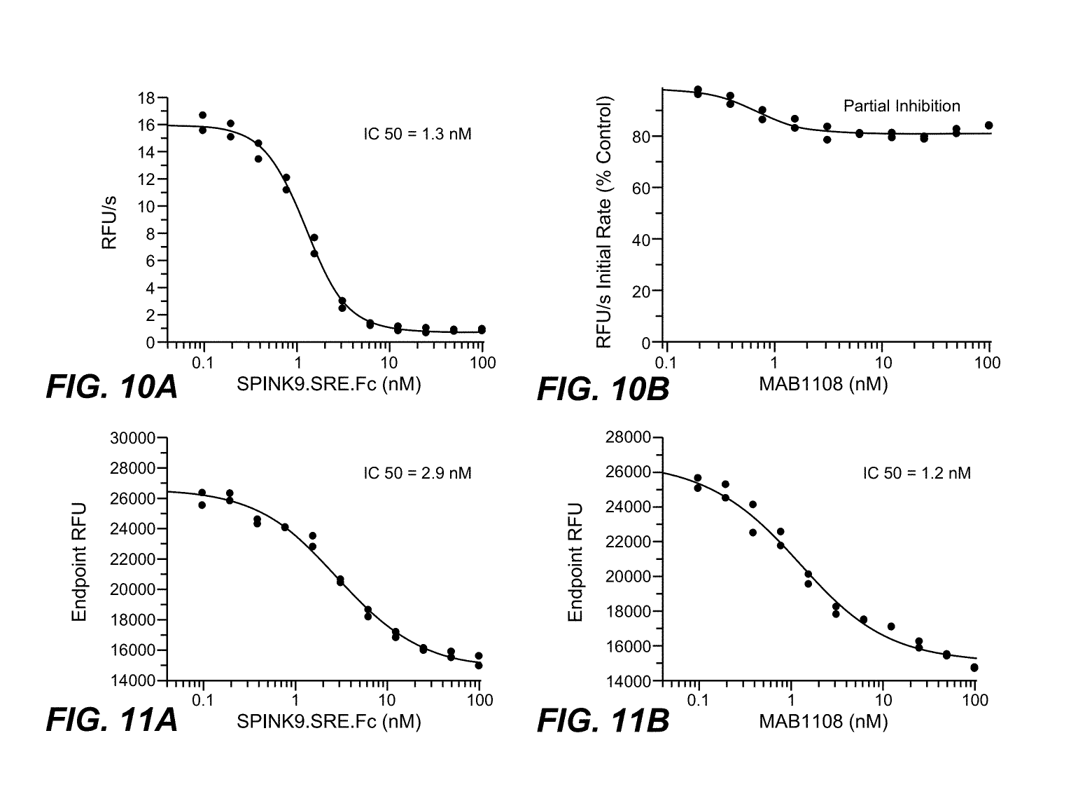

To further evaluate the inhibition profile of mAb1108, the commercial antibody was assayed against the SPINK9 Fc fusion polypeptide in both the direct (FIG. 6) and pro-KLK7 coupled (FIG. 7) assay. In the direct assay (FIG. 10), SPINK9 Fc fusion polypeptide was a potent inhibitor of KLK5 cleavage of the fluorescent peptide substrate, while mAb1108 demonstrated partial inhibition. Using the macromolecular substrate, pro-KLK7, in the coupled assay (FIG. 11), both SPINK9 Fc fusion polypeptide and mAb1108 were potent inhibitors of KLK5 activity. Taken together, these data indicate that while mAb1108 does demonstrate partial inhibition of KLK5 in the direct assay (FIGS. 8 and 9) and full inhibition in the coupled assay (FIG. 11B), the SPINK9 Fc fusion polypeptide is a potent inhibitor of KLK5 in both direct (FIGS. 6 and 10) and coupled assays (FIGS. 7 and 11).

### Example 4—KLK5-Derived Cleavage Peptide Detection by LC/MS for IC₅₀ Determination

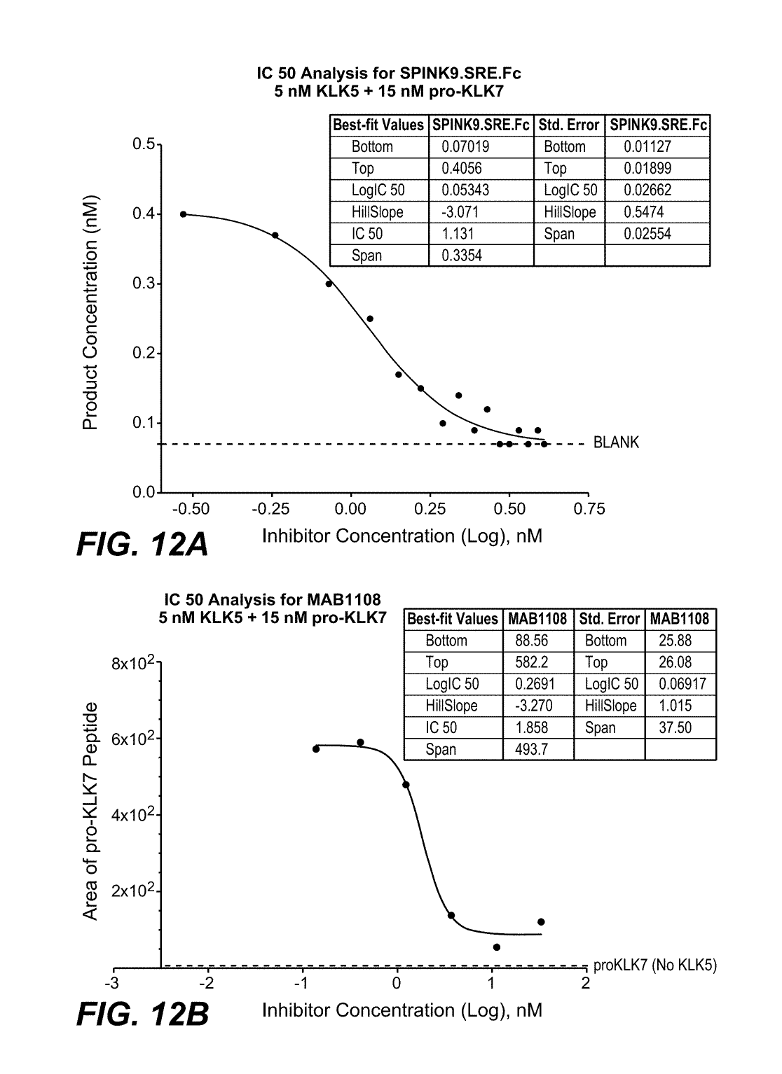

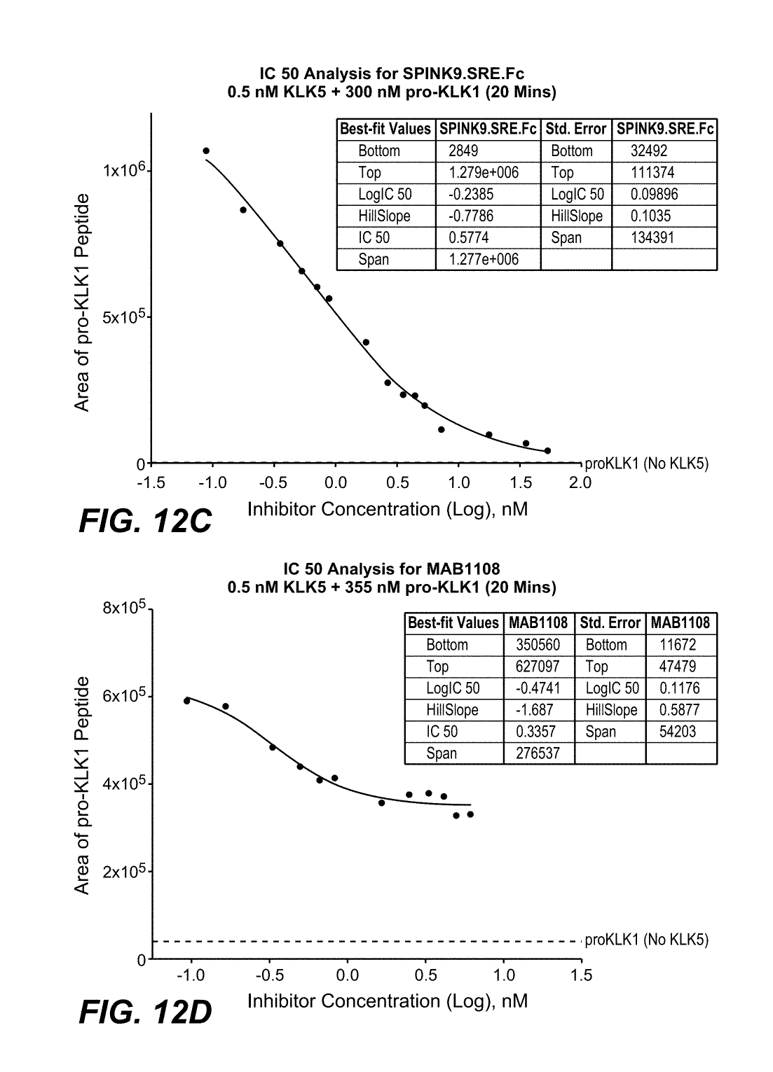

The ability of SPINK9.SRE.Fc and mAb1108 to inhibit proteolysis of pro-KLK7 or pro-KLK1 by recombinant KLK5 was assessed using an LC/MS assay that monitors the KLK5-derived cleavage product peptides. In the KLK7 assay, SPINK9.SRE.Fc and mAb1108 fully inhibit KLK5 (5 nM) cleavage of pro-KLK7 with IC₅₀ values of 1.13 nM (FIG. 12A) or 1.86 nM, respectively (FIG. 12 B). Whereas in the KLK1 assay, although SPINK9.SRE.Fc and mAb1108 both inhibit KLK5 (0.5 nM) cleavage of pro-KLK1, only SPINK9.SRE.Fc fully inhibits KLK5 with an IC₅₀ of 0.58 nM (FIG. 12C) while mAb1108 exhibits a maximum of ˜40% KLK5 inhibition with an IC₅₀ of 0.34 nM (FIG. 12 D).

### CONCLUSIONS

Taken together, these data suggested that KLK5 induces epithelial production of neutrophil chemotactic cytokines and neutrophil influx into the lung tissues. Herein are provided results from the first GWAS to focus specifically on periostin low, or type 2 low inflammation asthmatics. A SNP at the KLK4/5 locus was identified that was protective for asthma risk in the periostin low asthma population. This finding was also seen in eosinophil low asthmatics. The kallikrein locus at 19q13 has been previously associated with asthma via linkage studies and GWAS. See Myers et al., J Allergy Clin Immunol 130, 1294-1301 (2012). The SNP identified via GWAS, rs1061477, resides in intron 2 of KLK3 which was located approximately 63 kb 3′ of SNP rs117639512. These SNPs were not in linkage disequilibrium with each other (r²=0.004, D′=0.293). Others have looked at the genetics of eosinophil level. See Gudbjartsson et al., Nat Genet 41, 342-347 (2009). SNPs at the IL13 and IT 33 loci were found to be associated with eosinophil level. These were also well replicated asthma risk loci. However, no SNPs within 1 MB of the KLK4/5 region were identified as reaching suggestive significance in either study. This shows that this locus may be specific to periostin low, or type 2 low asthma.

SNP rs117639512 was intragenic, located between KLK4 and KLK5. Due to the relatively low frequency of this SNP the function on KLK4 was unable to be tested in online databases, and no statistical significance with regards to differing mRNA level of KLK5 could be observed. However, in the majority of tissues tested, a similar direction of effect resulting in lower mRNA levels of KLK5 for carriers of the minor allele was seen. This combined with the protective OR see for the SNP, indicate that lower levels of KLK5 were protective from asthma risk. This is consistent with the findings from Netherton syndrome patients, where severely upregulated levels of KLK5 result in many atopic phenotypes, including asthma. Netherton syndrome was associated with loss of function mutations in the KLK5 regulator SPINK5. The GTEx database for SNPs affecting mRNA levels of SPINK5 was assessed. The strongest hit, at SNP rs1363727, was associated with significantly lower SPINK5 mRNA levels in the GTEx database for alternate allele carriers (P<1.2×10⁻⁸ in 10 tissues). This SNP was tested in the periostin low asthma population and it did not reach statistical significance for asthma risk (P=0.063; OR=1.14), but had an opposite direction of effect compared to the KLK4/KLK5 locus SNP. This indicates that lower levels of SPINK5 may increase risk of developing asthma. The reduced function of SPINK5 and increased activity of KLK5 is consistent with the findings from Netherton syndrome. Thus, the genetic evidence suggests that lowering KLK5 levels may be protective for asthma outside of the context of Netherton syndrome.

KLK5 binding polypeptide levels were elevated in bronchial alveolar lavage fluid of severe asthma patients, and correlated negatively with predicted FEV1 (p<0.05), supporting the hypothesis that KLK5 may play a pathogenic role in bronchial obstruction and asthma pathogenesis. The regulation of KLK5 in asthma as well as other allergic diseases remains unclear. There was no correlation between KLK5 and type 2 inflammation biomarkers (e.g., periostin, FeNO, and blood eosinophil counts). KLK5 was mainly expressed by lung epithelium. Asthma patients have frequent injury and loss of epithelium barrier, which is associated with the regeneration process involving the induced growth factors, repair processing, and tissue remodeling. Dysregulated epithelial cell activation, regeneration process, and tissue remodeling in the severe asthma may attribute to the abnormal KLK5 level in the asthmatic lung compartment.

SPINK5 is a natural reversible inhibitor for KLK5 through direct binding to its catalytic active site. SPINK5 is expressed by many mucosal tissues, including skin, lung, esophagus, and gastrointestinal tract. In Nertherton syndrome, deficiency of SPINK5 leads to increased KLK5 activity, cutaneous inflammation, and allergic symptoms. See Briot et al., J Exp Med 206, 1135-1147 (2009). It was found that SPINK5 is directly induced by inflammatory cytokines, particularly interleukin IL-13 (data not shown). In line with this observation, SPINK5 transcript was reduced in Th2-low asthma patients as compared with Th2-high asthma patients. The higher ratio of KLK5/SPINK5, primarily driven by reduced SPINK5 expression, may contribute to the asthma pathology in Th2-low asthma patients.

A recombinant form of KLK5 was generated and found that a small amount of enzymatically active KLK5 potently induced neutrophil influx into the bronchial alveolar lavage and lung tissue. As neutrophils do not extravasate into the lungs of animals given catalytically inactive KLK5 (or heat inactivated KLK5, data not shown), the catalytic activity is essential for neutrophil recruitment. This was consistent with reports KLK5 transgenic mice have massive neutrophil infiltration in the skin lesions. See Furio et al., J Exp Med 211, 499-513 (2014). KLK5 induces epithelial expression of inflammation cytokines, chemokines, and adhesion molecules. In particular, IL-8 is a critical neutrophil chemotactic cytokine. ICAM-1 was critical adhesion molecule for neutrophil adhesion through its interaction with CD11b/CD18 integrins. TNF-α induces vascular leakage and promotes cellular extravasation into the peripheral tissues. Inflammatory cytokines, chemokines, and adhesion molecules induced by KLK5 may work together to promote neutrophil influx into the local tissues. The rapid induction of inflammatory chemokine/cytokines indicates that cell surface receptor(s) may be present to mediate the cell signaling events.

In summary, provided herein are data showing a genetic association with a SNP at the KLK5 locus with asthma risk that was specific to periostin low, or type-2 low inflammation asthma cases. Furthermore, data presented describes an effect of KLK5 on asthma symptoms and sub-phenotypes. Results presented herein suggest that reducing KLK5 activity may have a protective effect on asthma.

>sp|Q9Y337|KLK5_HUMAN Kallikrein-5 OS = Homo
sapiens GN = KLK5 PE = 1 SV = 2, (full-length
KLK5 including signal peptide amino acids 1-22
underlined)
SEQ ID NO: 1
MATARPPWMWVLCALITALLLGVTEHVLANNDVSCDHPSNTVPSGSNQ
DLGAGAGEDARSDDSSSRIINGSDCDMHTQPWQAALLLRPNQLYCGAV
LVHPQWLLTAAHCRKKVFRVRLGHYSLSPVYESGQQMFQGVKSIPHPG
YSHPGHSNNLMLIKLNRRIRPTKDVRPINVSSHCPSAGTKCLVSGWGT
TKSPQVHFPKVLQCLNISVLSQKRCEDAYPRQIDDTMFCAGDKAGRDS
CQGDSGGPVVCNGSLQGLVSWGDYPCARPNRPGVYTNLCKFTKWIQET
IQANS
Mature Form of KLK5 (minus signal peptide amino
acids 1-22)
SEQ ID NO: 2
VTEHVLANNDVSCDHPSNTVPSGSNQDLGAGAGEDARSDDSSSRIING
SDCDMHTQPWQAALLLRPNQLYCGAVLVHPQWLLTAAHCRKKVFRVRL
GHYSLSPVYESGQQMFQGVKSIPHPGYSHPGHSNNLMLIKLNRRIRPT
KDVRPINVSSHCPSAGTKCLVSGWGTTKSPQVHFPKVLQCLNISVLSQ
KRCEDAYPRQIDDTMFCAGDKAGRDSCQGDSGGPVVCNGSLQGLVSWG
DYPCARPNRPGVYTNLCKFTKWIQETIQANS
|KLK5_HUMAN Kallikrein-5 (N153D variant of
full-length KLK5 including signal peptide amino
acids 1-22 underlined)
SEQ ID NO: 3
MATARPPWMWVLCALITALLLGVTEHVLANNDVSCDHPSNTVPSGSNQ
DLGAGAGEDARSDDSSSRIINGSDCDMHTQPWQAALLLRPNQLYCGAV
LVHPQWLLTAAHCRKKVFRVRLGHYSLSPVYESGQQMFQGVKSIPHPG
YSHPGHSNDLMLIKLNRRIRPTKDVRPINVSSHCPSAGTKCLVSGWGT
TKSPQVHFPKVLQCLNISVLSQKRCEDAYPRQIDDTMFCAGDKAGRDS
CQGDSGGPVVCNGSLQGLVSWGDYPCARPNRPGVYTNLCKFTKWIQET
IQANS
Mature Form of KLK5 (N153D variant, minus signal
peptide amino acids 1-22)
SEQ ID NO: 4
VTEHVLANNDVSCDHPSNTVPSGSNQDLGAGAGEDARSDDSSSRIING
SDCDMHTQPWQAALLLRPNQLYCGAVLVHPQWLLTAAHCRKKVFRVRL
GHYSLSPVYESGQQMFQGVKSIPHPGYSHPGHSNDLMLIKLNRRIRPT
KDVRPINVSSHCPSAGTKCLVSGWGTTKSPQVHFPKVLQCLNISVLSQ
KRCEDAYPRQIDDTMFCAGDKAGRDSCQGDSGGPVVCNGSLQGLVSWG
DYPCARPNRPGVYTNLCKFTKWIQETIQANS
|KLK5_HUMAN Kallikrein-5 (G55R variant of
full-length KLK5 including signal peptide amino
acids 1-22 underlined)
SEQ ID NO: 5
MATARPPWMWVLCALITALLLGVTEHVLANNDVSCDHPSNTVPSGSNQ
DLGAGAREDARSDDSSSRIINGSDCDMHTQPWQAALLLRPNQLYCGAV
LVHPQWLLTAAHCRKKVFRVRLGHYSLSPVYESGQQMFQGVKSIPHPG
YSHPGHSNNLMLIKLNRRIRPTKDVRPINVSSHCPSAGTKCLVSGWGT
TKSPQVHFPKVLQCLNISVLSQKRCEDAYPRQIDDTMFCAGDKAGRDS
CQGDSGGPVVCNGSLQGLVSWGDYPCARPNRPGVYTNLCKFTKWIQET
IQANS
Mature Form of KLK5 (G55R variant, minus signal
peptide amino acids 1-22)
SEQ ID NO: 6
VTEHVLANNDVSCDHPSNTVPSGSNQDLGAGAREDARSDDSSSRIING
SDCDMHTQPWQAALLLRPNQLYCGAVLVHPQWLLTAAHCRKKVERVRL
GHYSLSPVYESGQQMFQGVKSIPHPGYSHPGHSNNLMLIKLNRRIRPT
KDVRPINVSSHCPSAGTKCLVSGWGTTKSPQVHFPKVLQCLNISVLSQ
KRCEDAYPRQIDDTMFCAGDKAGRDSCQGDSGGPVVCNGSLQGLVSWG
DYPCARPNRPGVYTNLCKFTKWIQETIQANS
|KLK5_HUMAN Kallikrein-5 (G55R, N153D variant
of full-length KLK5 including signal peptide
amino acids 1-22 underlined)
SEQ ID NO: 7
MATARPPWMWVLCALITALLLGVTEHVLANNDVSCDHPSNTVPSGSNQ
DLGAGAREDARSDDSSSRIINGSDCDMHTQPWQAALLLRPNQLYCGAV
LVHPQWLLTAAHCRKKVFRVRLGHYSLSPVYESGQQMFQGVKSIPHPG
YSHPGHSNDLMLIKLNRRIRPTKDVRPINVSSHCPSAGTKCLVSGWGT
TKSPQVHFPKVLQCLNISVLSQKRCEDAYPRQIDDTMFCAGDKAGRDS
CQGDSGGPVVCNGSLQGLVSWGDYPCARPNRPGVYTNLCKFTKWIQET
IQANS
Mature Form of KLK5 (G55R, N153D variant, minus
signal peptide amino acids 1-22)
SEQ ID NO: 8
VTEHVLANNDVSCDHPSNTVPSGSNQDLGAGAREDARSDDSSSRIING
SDCDMHTQPWQAALLLRPNQLYCGAVLVHPQWLLTAAHCRKKVERVRL
GHYSLSPVYESGQQMFQGVKSIPHPGYSHPGHSNDLMLIKLNRRIRPT
KDVRPINVSSHCPSAGTKCLVSGWGTTKSPQVHFPKVLQCLNISVLSQ
KRCEDAYPRQIDDTMFCAGDKAGRDSCQGDSGGPVVCNGSLQGLVSWG
DYPCARPNRPGVYTNLCKFTKWIQETIQANS
>sp|Q9NQ38|ISK5_HUMAN Serine protease inhibitor
Kazal-type 5 OS = Homo sapiens GN = SPINK5
PE = 1 SV = 2 (full-length human SPINK5
including signal peptide amino acids 1-22
underlined)
SEQ ID NO: 9
MKIATVSVLLPLALCLIQDAASKNEDQEMCHEFQAFMKNGKLFCPQDK
KFFQSLDGIMFINKCATCKMILEKEAKSQKRARHLARAPKATAPTELN
CDDFKKGERDGDFICPDYYEAVCGTDGKTYDNRCALCAENAKTGSQIG
VKSEGECKSSNPEQDVCSAFRPFVRDGRLGCTRENDPVLGPDGKTHGN
KCAMCAELFLKEAENAKREGETRIRRNAEKDFCKEYEKQVRNGRLFCT
RESDPVRGPDGRMHGNKCALCAEIFKQRFSEENSKTDQNLGKAEEKTK
VKREIVKLCSQYQNQAKNGILFCTRENDPIRGPDGKMHGNLCSMCQAY
FQAENEEKKKAEARARNKRESGKATSYAELCSEYRKLVRNGKLACTRE
NDPIQGPDGKVHGNTCSMCEVFFQAEEEEKKKKEGKSRNKRQSKSTAS
FEELCSEYRKSRKNGRLFCTRENDPIQGPDGKMHGNTCSMCEAFFQQE
ERARAKAKREAAKEICSEFRDQVRNGTLICTREHNPVRGPDGKMHGNK
CAMCASVFKLEEEEKKNDKEEKGKVEAEKVKREAVQELCSEYRHYVRN
GRLPCTRENDPIEGLDGKIHGNTCSMCEAFFQQEAKEKERAEPRAKVK
REAEKETCDEFRRLLQNGKLFCTRENDPVRGPDGKTHGNKCAMCKAVF
QKENEERKRKEEEDQRNAAGHGSSGGGGGNTQDECAEYREQMKNGRLS
CTRESDPVRDADGKSYNNQCTMCKAKLEREAERKNEYSRSRSNGTGSE
SGKDTCDEFRSQMKNGKLICTRESDPVRGPDGKTHGNKCTMCKEKLER
EAAEKKKKEDEDRSNTGERSNTGERSNDKEDLCREFRSMQRNGKLICT
RENNPVRGPYGKMHINKCAMCQSIFDREANERKKKDEEKSSSKPSNNA
KDECSEFRNYIRNNELICPRENDPVHGADGKFYTNKCYMCRAVFLTEA
LERAKLQEKPSHVRASQEEDSPDSFSSLDSEMCKDYRVLPRIGYLCPK
DLKPVCGDDGQTYNNPCMLCHENLIRQTNTHIRSTGKCEESSTPGTTA
ASMPPSDE
Mature Form of human SPINK5 (minus signal
peptide amino acids 1-22)
SEQ ID NO: 10
KNEDQEMCHEFQAFMKNGKLFCPQDKKFFQSLDGIMFINKCATCKMIL
EKEAKSQKRARHLARAPKATAPTELNCDDFKKGERDGDFICPDYYEAV
CGTDGKTYDNRCALCAENAKTGSQIGVKSEGECKSSNPEQDVCSAFRP
FVRDGRLGCTRENDPVLGPDGKTHGNKCAMCAELFLKEAENAKREGET
RIRRNAEKDFCKEYEKQVRNGRLFCTRESDPVRGPDGRMHGNKCALCA
EIFKQRFSEENSKTDQNLGKAEEKTKVKREIVKLCSQYQNQAKNGILF
CTRENDPIRGPDGKMHGNLCSMCQAYFQAENEEKKKAEARARNKRESG
KATSYAELCSEYRKLVRNGKLACTRENDPIQGPDGKVHGNTCSMCEVF
FQAEEEEKKKKEGKSRNKRQSKSTASFEELCSEYRKSRKNGRLFCTRE
NDPIQGPDGKMHGNTCSMCEAFFQQEERARAKAKREAAKEICSEFRDQ
VRNGTLICTREHNPVRGPDGKMHGNKCAMCASVFKLEEEEKKNDKEEK
GKVEAEKVKREAVQELCSEYRHYVRNGRLPCTRENDPIEGLDGKIHGN
TCSMCEAFFQQEAKEKERAEPRAKVKREAEKETCDEFRRLLQNGKLFC
TRENDPVRGPDGKTHGNKCAMCKAVFQKENEERKRKEEEDQRNAAGHG
SSGGGGGNTQDECAEYREQMKNGRLSCTRESDPVRDADGKSYNNQCTM
CKAKLEREAERKNEYSRSRSNGTGSESGKDTCDEFRSQMKNGKLICTR
ESDPVRGPDGKTHGNKCTMCKEKLEREAAEKKKKEDEDRSNTGERSNT
GERSNDKEDLCREFRSMQRNGKLICTRENNPVRGPYGKMHINKCAMCQ
SIFDREANERKKKDEEKSSSKPSNNAKDECSEFRNYIRNNELICPREN
DPVHGADGKFYTNKCYMCRAVFLTEALERAKLQEKPSHVRASQEEDSP
DSFSSLDSEMCKDYRVLPRIGYLCPKDLKPVCGDDGQTYNNPCMLCHE
NLIRQTNTHIRSTGKCEESSTPGTTAASMPPSDE
>tr|Q5K5D4|Q5K5D4_MOUSE Spink5 protein
OS = Mus musculus GN = Spink5 PE = 2 SV = 1
(full-length mouse SPINK5 including signal
peptide amino acids 1-22 underlined)
SEQ ID NO: 11
MKTATVPMLLTLAFYLTQDAAGEKGNQDPCMKFQAQMKNGTLTCPKGN
NSSQSLNDIIFQSECILCKRALEQGAPTKIMNVKVLSRANRATDPAKL
NCESFKQRRKDGDFICPSDTSSVCGTDGKTYRGRCELCAENAKSQNHV
DVKSEGECGSSHLETDMCSDFRANVQDGRLGCTRESDPILGPDGRTHG
NRCAMCAELFLKEAKENATRNRESRIRRDAEKELCKEFENQVRNGRLF
CTRESDPIRGPDGKMHGNKCALCAEIFMRQFTEEKGKAEKNQKDAEER
AKAKMEIQKRCSEFQDRARNGTLFCTRENDPIRGLDGKTHGNLCSMCQ
AFFKTEAEEKKAEAGSRNRRGSEESETYAKLCDEYRKARKNGQLYCTR
ENAPIRGPDGKIHGNTCSMCQAFFIQEDKARAKVKREAAKEMCSEFRN
QARNGMLMCTRENDPVVGPDGKRHSNKCAMCASVFLLEEEEKKKDDKT
EKVDAGKAKKEAVQELCRKYHTQLRNGPLRCTRRNNPIEGLDGKMYKN
ACFMCWAFFQQEAKKSGAGFRPKVKREVKVDCSEYLALSKRGEIFCTR
ENDPVRGPDGKTHGNKCAMCKAVFKKENEERKRKEGENQRITSGESSS
GGNPKAKDECAQYRESMKHGQLSCTRESDPVRGVDGEHYNNKCVMCKE
LLQKEMEETNKNSASRSNGTGSATGKDVCDQFRSQMKNGKLLCTRESD
PTRGPDGAMHGNKCAMCKERLEKEAAEKKKKEDEEKRNTETNKSDKED
KCHEYRSMQLDGRLICTRENDPVRDADGKMHVNKCAMCQMMFEREANE
RKMREENSRSQPTNEAKDQCGEVHNSVEDAKPRPARSSLPSIRGISKD
ECSEFQNLMKNEKLTCPETDDPVRGADGTFYQNKCHMCRDVLKNEAMK
RSGLQEKSSDIRSTKEGDPEFSSSSRDSDMCKNYRILPRMGYLCPKNL
NPVCGDDGQTYSNPCMLCHENLMRQTNTRIHNPGACEESSNLKTVSTG
TPASEKMMQ
Mature Form of mouse SPINK5 (minus signal
peptide amino acids 1-22)
SEQ ID NO: 12
EKGNQDPCMKFQAQMKNGTLTCPKGNNSSQSLNDIIFQSECILCKRAL
EQGAPTKIMNVKVLSRANRATDPAKLNCESFKQRRKDGDFICPSDTSS
VCGTDGKTYRGRCELCAENAKSQNHVDVKSEGECGSSHLETDMCSDFR
ANVQDGRLGCTRESDPILGPDGRTHGNRCAMCAELFLKEAKENATRNR
ESRIRRDAEKELCKEFENQVRNGRLFCTRESDPIRGPDGKMHGNKCAL
CAEIFMRQFTEEKGKAEKNQKDAEERAKAKMEIQKRCSEFQDRARNGT
LFCTRENDPIRGLDGKTHGNLCSMCQAFFKTEAEEKKAEAGSRNRRGS
EESETYAKLCDEYRKARKNGQLYCTRENAPIRGPDGKIHGNTCSMCQA
FFIQEDKARAKVKREAAKEMCSEFRNQARNGMLMCTRENDPVVGPDGK
RHSNKCAMCASVFLLEEEEKKKDDKTEKVDAGKAKKEAVQELCRKYHT
QLRNGPLRCTRRNNPIEGLDGKMYKNACFMCWAFFQQEAKKSGAGFRP
KVKREVKVDCSEYLALSKRGEIFCTRENDPVRGPDGKTHGNKCAMCKA
VFKKENEERKRKEGENQRITSGESSSGGNPKAKDECAQYRESMKHGQL
SCTRESDPVRGVDGEHYNNKCVMCKELLQKEMEETNKNSASRSNGTGS
ATGKDVCDQFRSQMKNGKLLCTRESDPTRGPDGAMHGNKCAMCKERLE
KEAAEKKKKEDEEKRNTETNKSDKEDKCHEYRSMQLDGRLICTRENDP
VRDADGKMHVNKCAMCQMMFEREANERKMREENSRSQPTNEAKDQCGE
VHNSVEDAKPRPARSSLPSIRGISKDECSEFQNLMKNEKLTCPETDDP
VRGADGTFYQNKCHMCRDVLKNEAMKRSGLQEKSSDIRSTKEGDPEFS
SSSRDSDMCKNYRILPRMGYLCPKNLNPVCGDDGQTYSNPCMLCHENL
MRQTNTRIHNPGACEESSNLKTVSTGTPASEKMMQ
(Hu SPINK5 (E490-Y757, Kazal domain D8-D11;
Double Underlined: Linker; Underlined: Fc human
IgG1 F356.M358)
SEQ ID NO: 13
EAAKEICSEFRDQVRNGTLICTREHNPVRGPDGKMHGNKCAMCASVFK
LEEEEKKNDKEEKGKVEAEKVKREAVQELCSEYRHYVRNGRLPCTREN
DPIEGLDGKIHGNTCSMCEAFFQQEAKEKERAEPRAKVKREAEKETCD
EFRRLLQNGKLFCTRENDPVRGPDGKTHGNKCAMCKAVFQKENEERKR
KEEEDQRNAAGHGSSGGGGGNTQDECAEYREQMKNGRLSCTRESDPVR
DADGKSYNNQCTMCKAKLEREAERKNEYGNSVTDKTHTCPPCPAPELL
GGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSV
MHEALHNHYTQKSLSLSPGK
SEQ ID NO: 14; (Hu SPINK5 (E490-Y757, Kazal
domain D8-D11; Double Underlined: Linker;
Underlined: Fc human IgG4.S228P)
EAAKEICSEFRDQVRNGTLICTREHNPVRGPDGKMHGNKCAMCASVFK
LEEEEKKNDKEEKGKVEAEKVKREAVQELCSEYRHYVRNGRLPCTREN
DPIEGLDGKIHGNTCSMCEAFFQQEAKEKERAEPRAKVKREAEKETCD
EFRRLLQNGKLFCTRENDPVRGPDGKTHGNKCAMCKAVFQKENEERKR
KEEEDQRNAAGHGSSGGGGGNTQDECAEYREQMKNGRLSCTRESDPVR
DADGKSYNNQCTMCKAKLEREAERKNEYGNSVTSKYGPPCPPCPAPEF
LGGPSVFLFPPKPKDTLMISRTPEVTCVVVDVSQEDPEVQFNWYVDGV
EVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCS
VMHEALHNHYTQKSLSLSLGK
(Hu SPINK5 (E490-Y757, Kazal domain D8-D11)
SEQ ID NO: 15
EAAKEICSEFRDQVRNGTLICTREHNPVRGPDGKMHGNKCAMCASVFK
LEEEEKKNDKEEKGKVEAEKVKREAVQELCSEYRHYVRNGRLPCTREN
DPIEGLDGKIHGNTCSMCEAFFQQEAKEKERAEPRAKVKREAEKETCD
EFRRLLQNGKLFCTRENDPVRGPDGKTHGNKCAMCKAVFQKENEERKR
KEEEDQRNAAGHGSSGGGGGNTQDECAEYREQMKNGRLSCTRESDPVR
DADGKSYNNQCTMCKAKLEREAERKNEY
(Mu SPINK5 (E421-A695)-Fc, (Kazal domain D6-D9;
Double underlined: Linker; Underlined: Fc mouse
IgG2a)
SEQ ID NO: 16
EAAKEMCSEFRNQARNGMLMCTRENDPVVGPDGKRHSNKCAMCASVFL
LEEEEKKKDDKTEKVDAGKAKKEAVQELCRKYHTQLRNGPLRCTRRNN
PIEGLDGKMYKNACFMCWAFFQQEAKKSGAGFRPKVKREVKVDCSEYL
ALSKRGEIFCTRENDPVRGPDGKTHGNKCAMCKAVFKKENEERKRKEG
ENQRITSGESSSGGNPKAKDECAQYRESMKHGQLSCTRESDPVRGVDG
EHYNNKCVMCKELLQKEMEETNKNSASRSNGTGSAGNSRAQVTDKKIE
PRGPTIKPCPPCKCPAPNLLGGPSVFIFPPKIKDVLMISLSPIVTCVV
VDVSEDDPDVQISWFVNNVEVHTAQTQTHREDYNSTLRVVSALPIQHQ
DWMSGKEFKCKVNNKDLPAPIERTISKPKGSVRAPQVYVLPPPEEEMT
KKQVTLTCMVTDFMPEDIYVEWTNNGKTELNYKNTEPVLDSDGSYFMY
SKLRVEKKNWVERNSYSCSVVHEGLHNHHTTKSFSRTPGK
(Mu SPINK5 (E421-A695, Kazal domain D6-D9)
SEQ ID NO: 17
EAAKEMCSEFRNQARNGMLMCTRENDPVVGPDGKRHSNKCAMCASVFL
LEEEEKKKDDKTEKVDAGKAKKEAVQELCRKYHTQLRNGPLRCTRRNN
PIEGLDGKMYKNACFMCWAFFQQEAKKSGAGFRPKVKREVKVDCSEYL
ALSKRGEIFCTRENDPVRGPDGKTHGNKCAMCKAVFKKENEERKRKEG
ENQRITSGESSSGGNPKAKDECAQYRESMKHGQLSCTRESDPVRGVDG
EHYNNKCVMCKELLQKEMEETNKNSASRSNGTGSA
(Hu SPINK5 (R291-R352; Kazal domain D5; Double
underlined: Linker; Underlined: Fc human IgG1
F356.M358)
SEQ ID NO: 18
REIVKLCSQYQNQAKNGILFCTRENDPIRGPDGKMHGNLCSMCQAYFQ
AENEEKKKAEARARGNSVTDKTHTCPPCPAPELLGGPSVFLFPPKPKD
TLMISRTPEVTCVVVDVSHEDPEVKFNWYVDGVEVHNAKTKPREEQYN
STYRVVSVLTVLHQDWLNGKEYKCKVSNKALPAPIEKTISKAKGQPRE
PQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSL
SLSPGK
(Hu SPINK5 (R291-R352; Kazal domain D5; Double
underlined: Linker; Underlined: Fc human
IgG4.S228P)
SEQ ID NO: 19
REIVKLCSQYQNQAKNGILFCTRENDPIRGPDGKMHGNLCSMCQAYFQ
AENEEKKKAEARARGNSVTSKYGPPCPPCPAPEFLGGPSVFLFPPKPK
DTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKS
LSLSLGK
(Hu SPINK5 (R291-R352; Kazal Domain D5)
SEQ ID NO: 20
REIVKLCSQYQNQAKNGILFCTRENDPIRGPDGKMHGNLCSMCQAYFQ
AENEEKKKAEARAR
(Mu SPINK5 (M293-R355; Kazal domain D4; Double
underlined: Linker; Underlined: Fc mouse IgG2a)
SEQ ID NO: 21
MEIQKRCSEFQDRARNGTLFCTRENDPIRGLDGKTHGNLCSMCQAFFK
TEAEEKKAEAGSRNRGNSRAQVTDKKIEPRGPTIKPCPPCKCPAPNLL
GGPSVFIFPPKIKDVLMISLSPIVTCVVVDVSEDDPDVQISWFVNNVE
VHTAQTQTHREDYNSTLRVVSALPIQHQDWMSGKEFKCKVNNKDLPAP
IERTISKPKGSVRAPQVYVLPPPEEEMTKKQVTLTCMVTDFMPEDIYV
EWTNNGKTELNYKNTEPVLDSDGSYFMYSKLRVEKKNWVERNSYSCSV
VHEGLHNHHTTKSFSRTPGK
(Mu SPINK5 (M293-R355; Kazal domain D4)
SEQ ID NO: 22
MEIQKRCSEFQDRARNGTLFCTRENDPIRGLDGKTHGNLCSMCQAFFK
TEAEEKKAEAGSRNR
>sp|Q5DT21|ISK9_HUMAN Serine protease inhibitor
Kazal-type 9 OS = Homo sapiens GN = SPINK9
PE = 1 SV = 1 (full-length human SPINK9
including signal peptide amino acids 1-19
underlined)
SEQ ID NO: 23
MRATAIVLLLALTLATMFSIECAKQTKQMVDCSHYKKLPPGQQRFCHH
MYDPICGSDGKTYKNDCFFCSKVKKTDGTLKFVHFGKC
Mature Form of human SPINK9 (minus signal
peptide amino acids 1-19)
SEQ ID NO: 24
IECAKQTKQMVDCSHYKKLPPGQQRFCHHMYDPICGSDGKTYKNDCFF
CSKVKKTDGTLKFVHFGKC
(Hu SPINK9 (I20-C86.C22S.H48R.M49E; Double
underlined: Linker; Underlined: Fe human IgG1
E356.M358)
SEQ ID NO: 25
IESAKQTKQMVDCSHYKKLPPGQQRFCHREYDPICGSDGKTYKNDCFF
CSKVKKTDGTLKFVHFGKCGNSVTDKTHTCPPCPAPELLGGPSVFLFP
PKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHY
TQKSLSLSPGK
(Hu SPINK9 (I20-C86.C22S.H48R.M49E; Double
underlined: Linker; Underlined: Fe human
IgG4.S228P)
SEQ ID NO: 26
IESAKQTKQMVDCSHYKKLPPGQQRFCHREYDPICGSDGKTYKNDCFF
CSKVKKTDGTLKFVHFGKCGNSVTSKYGPPCPPCPAPEFLGGPSVFLF
PPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKA
KGQPREPQVYTLPPSQEEMTKNQVSLTCLVKGFYPSDIAVEWESNGQP
ENNYKTTPPVLDSDGSFFLYSRLTVDKSRWQEGNVFSCSVMHEALHNH
YTQKSLSLSLGK
(Hu SPINK9 (I20-C86.C22S.H48R.M49E; Double
underlined: Linker; Underlined: Fe mouse IgG2a)
SEQ ID NO: 27
IESAKQTKQMVDCSHYKKLPPGQQRFCHREYDPICGSDGKTYKNDCFF
CSKVKKTDGTLKFVHFGKCGNSRAQVTDKKIEPRGPTIKPCPPCKCPA
PNLLGGPSVFIFPPKIKDVLMISLSPIVTCVVVDVSEDDPDVQISWFV
NNVEVHTAQTQTHREDYNSTLRVVSALPIQHQDWMSGKEFKCKVNNKD
LPAPIERTISKPKGSVRAPQVYVLPPPEEEMTKKQVTLTCMVTDFMPE
DIYVEWTNNGKTELNYKNTEPVLDSDGSYFMYSKLRVEKKNWVERNSY
SCSVVHEGLHNHHTTKSFSRTPGK
(Hu SPINK9 (I20-C86.C22S.H48R.M49E))
SEQ ID NO: 28
IESAKQTKQMVDCSHYKKLPPGQQRFCHREYDPICGSDGKTYKNDCFF
CSKVKKTDGTLKFVHFGKC

1. A method for treating asthma in a subject comprising administering an effective amount of a KLK5 antagonist to the subject.

2. A method of predicting the response of a subject suffering from asthma to a treatment comprising a KLK5 antagonist, the method comprising:
(a) measuring the KLK5 level in a biological sample from the subject,
(b) comparing the KLK5 level detected in the sample to a reference level, and
(c) predicting that the subject will respond to the treatment when the KLK5 level measured in the sample is elevated compared to the reference level and predicting that the subject will not respond to the treatment when the KLK level measured in the sample is reduced compared to the reference level.

3. A method of selecting a subject suffering from asthma for a treatment comprising a KLK5 antagonist, comprising determining the presence or absence of a genetic variation located in the KLK5 genomic sequence in a biological sample from the subject, wherein the presence of the genetic variation indicates that the subject is suitable for treatment with a KLK5 antagonist.

4. A method for detecting the presence or absence of a genetic variation in the KLK5 genomic sequence indicating that a subject suffering from asthma is suitable for treatment with a KLK5 antagonist, comprising:
(a) contacting a sample from the subject with a reagent capable of detecting the presence or absence of the genetic variation located in the KLK5 genomic sequence; and
(b) determining the presence or absence of the genetic variation, wherein the presence of the genetic variation indicates that the subject is suitable for treatment with a KLK5 antagonist.

5. The method of claim 1, wherein asthma is related to a genetic variation located in the KLK5 genomic sequence.

6. The method of claim 1, wherein asthma is associated with elevated levels of KLK5.

7. The method of claim 1, wherein asthma is associated with elevated levels of neutrophils.

8. The method of claim 1, wherein asthma is selected from the group consisting of type 2 low asthma, periostin low asthma and eosinophil low asthma.

9. The method of claim 1, wherein asthma is not associated with Netherton Syndrome.

10. The method of claim 1, wherein asthma is associated with reduced activity of SPINK5.

11. The method of claim 1, wherein asthma is not associated with one or more genetic variations in the gene encoding SPINK5 or a gene product thereof.

12.-14. (canceled)

15. The method of claim 1, wherein the KLK5 antagonist inhibits KLK5 by binding to the active site of KLK5.

16. The method of claim 1, wherein the KLK5 antagonist inhibits KLK5 by binding to a binding region comprising one or more of the amino acid residues of KLK5 selected from the group consisting of the amino acid residues at position 108, 147, 150, 153, 168 and 245 of full-length unprocessed KLK5.

17. The method of claim 1, wherein the KLK5 antagonist inhibits the serine protease activity of KLK5.

18. The method of claim 1, wherein the KLK5 antagonist is selected from the group consisting of an antibody, a binding polypeptide, a polynucleotide and a small molecule.

19. The method of claim 18, wherein the KLK5 antagonist is an antibody.

20. The method of claim 19, wherein the antibody is a monoclonal antibody.

21. The method of claim 19, wherein the antibody is a human, humanized, or chimeric antibody.

22. The method of claim 19, wherein the antibody is a full length IgG1 antibody.

23. The method of claim 18, wherein the binding polypeptide is an Fc fusion polypeptide.

24. The method of claim 23, wherein the Fc fusion polypeptide comprises one or more domains of SPINK5.

25. The method of claim 23, wherein the Fc fusion polypeptide comprises the amino acid sequence SEQ ID NO:16 or SEQ ID NO:21.

26. The method of claim 23, wherein the Fc fusion polypeptide comprises one domain of SPINK9.

27. The method of claim 23, wherein the Fc fusion polypeptide comprises the amino acid sequence SEQ ID NO:27.

28. The method of claim 18, wherein the small molecule is a protease inhibitor.

29. The method of claim 28, wherein the protease inhibitor is leupeptin.

30. (canceled)

31. A KLK5 antagonist for use in medical treatment or diagnosis including therapy and/or treating of asthma.

32. A SPINK Fc fusion polypeptide, wherein the SPINK Fc fusion polypeptide inhibits the activity of KLK5.

33.-37. (canceled)

38. A pharmaceutical formulation comprising a pharmaceutically active amount of a SPINK Fc fusion polypeptide according to claim 32 and a pharmaceutically acceptable carrier.

39. A SPINK Fc fusion polypeptide according to claim 32 for use in medical treatment or diagnosis including therapy and/or treating a disease associated with KLK5.

40. A method for treating a disease associated with KLK5 in a subject comprising administering an effective amount of an Fc fusion polypeptide according to claim 32 to the subject.

41.-44. (canceled)

45. The method of claim 1, wherein the subject is a human.
